(19) United States
(12) Patent Application Publication
(10) Pub. No.: US 2022/0091133 A1
(43) Pub. Date: Mar. 24, 2022

(54) METHODS AND ASSAYS FOR IMMUNE PHENOTYPING

(71) Applicants: Washington University, St. Louis, MO (US); University of Cincinnati, Cincinnati, OH (US); University of Florida Research Foundation, Incorporated, Gainsville, FL (US)

(72) Inventors: Richard Hotchkiss, St. Louis, MO (US); Isaiah Turnbull, St. Louis, MO (US); Monty Mazer, St. Louis, MO (US); Kenneth Remy, St. Louis, MO (US); Charles Caldwell, Cincinnati, OH (US); Lyle Moldawer, Gainesville, FL (US); Scott Brakenridge, Seattle, WA (US)

(21) Appl. No.: 17/479,326

(22) Filed: Sep. 20, 2021

Related U.S. Application Data
(60) Provisional application No. 63/080,774, filed on Sep. 20, 2020.
(60) Provisional application No. 63/232,273, filed on Aug. 12, 2021.

(51) Int. Cl. G01N 33/68

(52) U.S. Cl. CPC G01N 33/6854; G01N 2333/57; G01N 2333/525; G01N 33/6893

(57) ABSTRACT

A method of immune phenotyping (evaluating adaptive and innate immune status) a subject is disclosed that includes providing a biological sample comprising diluted whole blood or isolated peripheral blood mononuclear cells (PBMCs), quantitating T cell interferon-gamma (IFN-γ) and monocyte TNF-α production using ELISpot in the biological sample comprising diluted whole blood, and determining that the subject has an immunosuppressive immunological endotype if T cell interferon-gamma (IFN-γ) and/or monocyte TNF-α production are decreased or low compared to a healthy subject. The disclosed method may be used to evaluating drug efficacy by measuring immune function in a subject after administering a drug to the subject to determine changes in the immune function of the subject in response to the drug.

### CROSS-REFERENCE TO RELATED APPLICATIONS

This application claims priority from U.S. Provisional Application Ser. No. 63/080,774 filed on 20 Sep. 2020 and 63/232,273 filed on 12 Aug. 2021, which are incorporated herein by reference in their entireties.

### STATEMENT REGARDING FEDERALLY SPONSORED RESEARCH OR DEVELOPMENT

This invention was made with government support under GM126928-01 awarded by the National Institutes of Health. The government has certain rights in the invention.

### MATERIAL INCORPORATED-BY-REFERENCE

Not applicable.

### FIELD

The present disclosure generally relates to methods and compositions for immune phenotyping patients.

### BACKGROUND

COVID-19-associated morbidity and mortality have been attributed to a pathologic host response. Two divergent hypotheses have been proposed: a hyper-inflammatory ‘cytokine-storm’-mediated injury versus failure of host protective immunity resulting in unrestrained viral dissemination and organ injury. A key explanation for the inability to address this controversy has been the lack of diagnostic tools to evaluate immune function in COVID-19 infections.

One of the most remarkable realities about the current SARS-CoV-2 infection outbreak (COVID-19) is that despite intense worldwide investigations, the decisive pathophysiologic processes that are responsible for patient morbidity and mortality remain unknown. Currently, the predominant paradigm is that an over-exuberant immune response mediated by excessive pro-inflammatory cytokines drives excessive lung injury and a pro-coagulant state. Accordingly, death is assumed to be primarily due to inflammatory lung injury, disturbances in micro- and macro-circulation, and resultant respiratory failure or vascular coagulopathy. This concept of a ‘cytokine storm’-mediated death in COVID-19 patients has been popularized in both the lay press and in many leading scientific publications. Based upon this theory, a number of anti-cytokine and anti-inflammatory therapies are being tested in COVID-19 including anti-IL-6(R) antibodies, IL-1 receptor antagonists, and JAK-STAT inhibitors, with early trial results failing to demonstrate significant efficacy.

Paradoxically, a second and diametrically opposed theory for COVID-19-induced morbidity and mortality is an ‘immunologic collapse’ of the host's protective system. This collapse of host protective immunity manifests itself as a failure to control unrestrained viral replication and dissemination with direct host cytotoxicity. Support for this contrasting theory is based upon the observed progressive and profound lymphopenia, often to numbers seen in patients with AIDS. Unlike the ‘cytokine storm’ which is often considered episodic, multiple recent studies show that lymphopenia is incessant in critically-ill COVID-19 patients and correlates with increased secondary infections and death. Postmortem studies of deceased COVID-19 patients have also identified a devastating loss of immune cells in spleen and secondary lymphoid organs. Multiple lymphocyte subsets are lost, including CD4 T, CD8 T, and NK cells that play vital antiviral roles, and in B cells that are essential for making antibodies that neutralize the virus.

Personalized medicine approaches require a better understanding regarding which of these immune endotypes predominate because the appropriate intervention is diametrically different depending upon whether the patient is suffering from hyper-inflammation or profound immunosuppression. For example, anti-IL-6(R) antibodies, IL-1 receptor antagonists and JAK-STAT inhibitors are currently undergoing clinical testing in COVID-19 patients and carry the potential to further compromise the patient's ability to eradicate the virus. Conversely, treatment with immune stimulants such as checkpoint inhibitors, IL-7, interferon-γ, or GM-CSF, currently either proposed or in active clinical trials in COVID-19, could exacerbate a dysfunctional and robust inflammatory response, and worsen organ injury.

Two distinct and key questions must be addressed in critically-ill COVID-19 patients: (1) what is their primary immune endotype, i.e., hyper-inflammatory versus immunosuppressive, and (2) how do these evolve over time with regards to disease progression or resolution. A better understanding of the COVID-19 patient's immune status would be instrumental in guiding proper immunotherapy.

There have been many efforts to immune endotype patients using genomic or proteomic biomarkers of immunity. While these methods have been helpful in predicting outcomes in sepsis and other disorders, in general, they have not been able to either provide an accurate assessment of the functional state of host immunity as it varies over time, or have been used to determine response to therapy.

### SUMMARY

Among the various aspects of the present disclosure is the provision of a highly sensitive, functional immunoassay, enzyme-linked immunosorbent spot (ELISpot) (ELISpot), that analyzes diluted whole blood to determine the immune status of a patient, i.e., whether the patient is in a more hyper-inflammatory phase or an immunosuppressive phase. The disclosed ELISpot assay may also be useful in guiding drug therapy to improve the immune function of the patient.

The present teachings include a method of immune phenotyping (evaluating adaptive and/or innate immune status) a subject that includes providing or having been provided a biological sample comprising diluted whole blood or isolated peripheral blood mononuclear cells (PBMCs), quantitating T cell interferon-gamma (IFN- [symbol] ) and/or monocyte TNF-α production using ELISpot in the biological sample comprising diluted whole blood, and/or determining that the subject has an immunosuppressive immunological endotype if T cell interferon-gamma (IFN- [symbol] ) and/or monocyte TNF-α production are low compared to a healthy subject. In accordance with another aspect, a method of evaluating drug efficacy by measuring immune function in a subject that includes providing or having been provided a biological sample comprising diluted whole blood or isolated peripheral blood mononuclear cells (PBMCs), quantitating T cell interferon-gamma (IFN- [symbol] ) and/or monocyte TNF-α production using ELISpot in the biological sample comprising diluted whole blood, determining that the subject has an immunosuppressive immunological endotype if T cell interferon-gamma (IFN- [symbol] ) and/or monocyte TNF-α production are low compared to a healthy subject, and/or administering a drug to the subject and/or determining the immune function of the subject in response to the drug.

An aspect of the present disclosure provides for a method of immune phenotyping a subject comprising: providing or having been provided a biological sample from the subject; optionally stimulating a T cell or monocyte cell or both to secrete a cytokine associated with cellular immunity; and/or quantitating at least one cytokine associated with cellular immunity using ELISpot assay or FluoroSpot assay in the biological sample. In some embodiments, the method further comprises determining that a subject has an immunosuppressive endotype if the cytokine associated with cellular immunity is a proinflammatory cytokine and/or proinflammatory cytokine production or secretion is decreased compared to a control. In some embodiments, the method further comprises determining that a subject has a hyper-inflammatory endotype if the cytokine associated with cellular immunity is a proinflammatory cytokine and/or the proinflammatory cytokine production or secretion is increased compared to a control. In some embodiments, the method further comprises determining if the subject has immunosuppressive endotype if immune cells amount is reduced compared to a control or hyper-inflammatory endotype if cytokine production is increased compared to a control. In some embodiments, the method further comprises detecting a level of innate immunity comprising detecting a level of blood monocytes or detecting a level low-density granulocytes or detecting a level of monocyte function or low-density granulocyte function. In some embodiments, the method further comprises detecting a level of adaptive cellular immunity comprising detecting a level of blood lymphocytes or blood lymphocytes function. In some embodiments, the subject has an immunosuppressive endotype if an amount of CD4⁺ and/or CD8⁺ T cells is reduced compared to a control, has reduced responsiveness of the T cells to T cell receptor activation, or both. In some embodiments, the cytokine associated with cellular immunity is a proinflammatory cytokine selected from the group consisting of T cell interferon-gamma (IFN- [symbol] ), monocyte tumor necrosis factor alpha (TNF-α), IL-1β, or combinations thereof. In some embodiments, the cytokine associated with cellular immunity is selected from IFN- [symbol] , TNF-α, IL-1β, IL-6, IL-7, IL-8, IL-10, IL-12, MCP-1, IL-1RA, or any combination thereof; or EGF, Eotaxin, FGF-basic, G-CSF, GM-CSF, HGF, IFN-α, IFN-γ, IL-1β, IL-1α, IL-1RA, IL-2, IL-2R, IL-3, IL-4, IL-5, IL-6, IL-7, IL-8, IL-9, IL-10, IL-12 (p40/p70) IL-13, IL-15, IL-17A, IL-17F, IL-22, IP-10, MCP-1, MIG, MIP-1α, MIP-1β, RANTES, TNF-α, VEGF, or any combination thereof. In some embodiments, quantitating cytokines associated with cellular immunity comprises: detecting an amount of cytokine-producing immune effector cells; or detecting an amount of cytokine produced on a cell. In some embodiments, quantitating cytokines associated with cellular immunity is measured in units of response per volume of blood. In some embodiments, the biological sample comprises: whole blood; diluted whole blood; circulating peripheral blood; whole blood diluted in about a 1:1 ratio with PBS; T cells, monocytes, and/or B cells; or plasma, leukocytes, red blood cells (RBCs), white blood cells (WBCs), platelets, cytokines, chemokines, or combinations thereof. In some embodiments, the biological sample does not comprise isolated peripheral blood mononuclear cells (PBMCs). In some embodiments, the method further comprises evaluating adaptive and/or innate immune status; evaluating monocyte or leukocyte function; evaluating progression of immune dysfunction in a subject; evaluating an effect of an immune therapy to restore innate and/or adaptive immunity in an immunosuppressed patient, optionally an immuno-adjuvant therapy to enhance host immunity; identifying optimal immune therapy for use in a subject; or improving immune function in a subject. In some embodiments, the subject has, is suspected of having, or is at risk for developing sepsis, autoimmune disease, autoimmunity, or cancer; the subject has Fungal Wound Sepsis; the subject has lymphopenia (≤1100 cells/μL); the subject has undergone organ transplantation; and/or the subject is in critical care. In some embodiments, the method further comprises measuring ex vivo cytokine production as a response to external stimuli. In some embodiments, the subject is septic or is determined to be at risk for premature death if: an amount of proinflammatory cytokine producing immune effector cells are decreased compared to a control; or an amount of proinflammatory cytokine produced per cell measured by spot intensity are decreased compared to a control. In some embodiments, if the subject does not have an immunosuppressive endotype or the subject has a hyper-inflammatory endotype, the subject is administered a drug that blocks proinflammatory cytokines or inhibits an inflammatory signaling cascade; if the subject has an immunosuppressive endotype, then the subject is administered IL-7 to restore disease-induced T cell exhaustion; if the subject has sepsis and/or has an immunosuppressive endotype, a drug restoring immunity is administered to the subject; if the subject is septic and/or immunosuppressed, then the subject is not administered corticosteroid therapy, optionally dexamethasone; the subject has sepsis and/or has the immunosuppressive endotype, the subject is at high risk for death; if the subject has the immunosuppressive endotype, the subject is treated with immuno-modulatory drug therapies or immune adjuvants that enhance host immunity; if the subject has an immunosuppressive endotype, then the subject is administered checkpoint inhibitors and/or common γ-chain cytokines that stimulate CD4 and/or CD8 T cells, optionally IL-17, if the subject has a hyper-inflammatory endotype or does not have an immunosuppressive endotype, the subject is treated with drugs to inhibit a host inflammatory response; if cytokine production in the subject is high, the subject is not treated with immunostimulant therapy; or if cytokine production in the subject is high, the subject is treated with anti-cytokine therapy or drugs to negatively modulate an inflammatory response. In some embodiments, the method further comprises detecting an immunosuppressive endotype or a hyper-inflammatory endotype during progression of a disease, disorder, or condition or during treatment of a disease, disorder, or condition. In some embodiments, the method further comprises administering a drug to a subject in need thereof and/or determining immune function or leukocyte function of the subject in response to the drug, optionally, during a course of immune therapy. In some embodiments, the subject has sepsis, COVID-19, cancer, trauma, or autoimmune disease; the subject is a critically ill nonseptic (CINS) or post-transplant patient; or the subject is immunosuppressed or a pediatric patient. In some embodiments, the biological sample is placed in fluid contact with a test therapeutic agent, optionally cytokines/chemokines, IL6, anti-PD-1, anti-PD-L1, GM512, CSF, IL-7. In some embodiments, the assay comprises a well pre-coated with a treatment directed at detecting one or more cytokines or chemokines.

Yet another aspect of the present disclosure provides for a method of screening a test therapeutic agent comprising: providing or having been provided immune cells; optionally determining if the immune cell has an immunosuppressive or hyper-inflammatory endotype; contacting the immune cell with a test therapeutic agent; and/or determining if one or more cytokines associated with cellular immunity are increased, decreased, or the same compared to a control or compared to before the immune cell was contacted with the test therapeutic agent. In some embodiments, the immune cell is a leukocyte, a monocyte, a T cell, or a combination thereof. In some embodiments, the test therapeutic agent is an immune adjuvant that selectively targets key immune effector cell types. In some embodiments, the test therapeutic agent is an immune adjuvant selected from anti-PD-1, anti-PD-L1, OX-40, GM-CSF, and/or IL-7. In some embodiments, the one or more cytokines associated with cellular immunity is T cell IFN-γ, monocyte TNF-α, or a combination thereof. In some embodiments, the immune cells are obtained from a subject having sepsis, COVID-19, cancer, trauma, autoimmune disease, ora critically ill nonseptic (CINS) or post-transplant patient.

Yet another aspect of the present disclosure provides for a method of evaluating drug efficacy by measuring immune function in a subject: providing or having been provided a biological sample comprising whole blood or diluted whole blood or isolated peripheral blood mononuclear cells (PBMCs); quantitating T cell interferon-gamma (IFN- [symbol] ) and/or monocyte TNF-α production using ELISpot in the biological sample comprising whole blood or diluted whole blood; optionally determining that a subject has an immunosuppressive endotype if T cell cytokine or monocyte cytokine production is low compared to a control; and/or administering a drug to the subject and/or determining the immune function of the subject in response to the drug. In some embodiments, the T cell cytokine is interferon-gamma (IFN- [symbol] ). In some embodiments, the monocyte cytokine is selected from one or more of TNF-α, IL-2, IL-6, and/or IL-12. In some embodiments, the subject has sepsis, COVID-19, cancer, trauma, or autoimmune disease; the subject is a critically ill nonseptic (CINS) or post-transplant patient; or the subject is immunosuppressed or a pediatric patient.

Yet another aspect of the present disclosure provides for an ELISpot or FluorSpot assay comprising wells, wherein the wells are precoated with one or more test therapeutic agents or one or more cytokine or chemokine detecting agents. In some embodiments, the one or more test therapeutic agents are tocilizumab, haptoglobin, hemopexin, ox40, IL7, or steroids. In some embodiments, the method further comprises a biological sample in fluid contact with the precoated wells, wherein the biological sample comprises whole blood, diluted whole blood, or isolated immune cells. In some embodiments, the biological sample is obtained from a subject having or suspected of having sepsis, COVID-19, cancer, trauma, or autoimmune disease; a critically ill nonseptic (CINS) subject or post-transplant patient; or an immunosuppressed or a pediatric patient. In some embodiments, the assay produces accelerated results compared to conventional PBMC assays.

Yet another aspect of the present disclosure provides for a method of reversing lymphopenia or improving T cell function in a subject comprising: providing or having been provided a biological sample from the subject; stimulating a T cell or monocyte cell or both to secrete a cytokine associated with cellular immunity; quantitating at least one cytokine associated with cellular immunity using ELISpot assay or FluoroSpot assay in the biological sample; and/or administering an immune-stimulating agent, optionally IL-7, GM-CSF, anti-PD-1, anti-PD-L1, or OX-40 agonistic Abs. In some embodiments, the subject has sepsis, COVID-19, cancer, trauma, or autoimmune disease; the subject is a critically ill nonseptic (CINS) or post-transplant patient; or the subject is immunosuppressed or a pediatric patient.

Yet another aspect of the present disclosure provides for a kit comprising an ELISpot or FluoroSpot assay comprising test agent-coated wells or wells coated with cytokine or chemokine detecting agents; and/or optionally a biological sample comprising whole blood or PBMCs.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. Graphical abstract of Covid-19 induced innate and adaptive immunosuppression.

FIG. 2. COVID-19 patient survival. Survival is plotted as a function from symptom onset (A) and ICU admission (B). Difference in ALC over time between survivors and nonsurvivors (C). Total patients n=27; survivors n=17, nonsurvivors n=10.

FIG. 3. Plasma IL-6 levels in patients with COVID-19 and sepsis. Dot plot representing plasma IL-6 levels for COVID-19 patients (A) and patients with sepsis (B) at various time points after ICU admission. Data bars represent mean±SEM. Red dots represent nonsurvivors. Septic patients n=10; COVID-19 days 1-3 n=19, days 4-7 n=17, days 8-11 n=8, days 12-15 n=8.

FIG. 4. Adaptive immune suppression in COVID-19 patients. Representative ELISpot photomicrographs displaying IFN- [symbol] production following overnight stimulation with anti-CD3/anti-CD28 antibodies for (A) healthy volunteers, (B) CINS patients, and (C) septic non-COVID-19 patients. (D) Three representative COVID-19-positive samples. Number of spots demonstrates the number of cytokine-producing T cells. Counts are presented as the corrected number of spots per thousand lymphocytes plated as fraction of the 2.5×10⁴ PBMCs plated in each well. Note the reduction in IFN- [symbol] production in both septic and COVID-19 patients compared with CINS patients. Note also a degree of heterogeneity in IFN- [symbol] production in COVID-19 and septic patients. Each photomicrograph was captured with the same magnification, and each image is to scale. ELISpot assays were performed using the PBMC fraction from freshly drawn whole blood. Each condition was run in duplicate for control samples and triplicate for COVID-19 samples.

FIG. 5. Functional immune cytokine production measured by ELISpot in COVID-19, CINS, and septic patients and healthy volunteers. Comparison graphs for ex vivo cytokine production using ELISpot, comparing healthy volunteers and CINS, septic, and COVID-19 patients. (A) Number of spots per 1000 lymphocytes plated following overnight culture stimulated with anti-CD3/anti-CD28 for IFN- [symbol] samples. (B) Number of spots per 1000 myeloid cells plated, stimulated with LPS for TNF-α production. Each dot represents an individual patient. Red dots represent nonsurvivors. Horizontal bars represent mean±SEM. Healthy n=27 for IFN- [symbol] , 28 for TNF-α; CINS n=18; septic n=46; COVID-19 n=25 for IFN- [symbol] , 24 for TNF-α. ANOVA comparing all groups for IFN-γ production showed that there was a difference between COVID-19 and the other groups (P=0.003); and for TNF-α groups there was a statistically significant difference as well (P=0.009). **P<0.01.

FIG. 6. Suppressed innate immune TNF-α response in COVID-19. Representative ELISpot photomicrographs displaying baseline innate immune (monocyte) function with LPS-stimulated TNF-α production in PBMCs. Comparison between different donor types, including (A) healthy control volunteers and (B) CINS, (C) septic, and (D) COVID-19 patients. Number of spots demonstrates the number of cytokine-producing monocytes, and counts are presented as corrected number of spots per thousand monocytes plated as fraction of the 2.5×10³ PBMCs plated in each well. COVID-19 patients had suppressed TNF-α production when compared with controls. Each photomicrograph was captured with the same magnification, and each image is to scale. ELISpot assays were performed using the PBMC fraction from freshly drawn whole blood. Each condition was run in duplicate for control samples and triplicate for COVID-19 samples.

FIG. 7. Number of cytokine-producing cells in COVID-19 patients serially over time. Time course analysis of ELISpot results comparing (A) IFN- [symbol] and (B) TNF-α production in COVID-19 survivors versus nonsurvivors (red) from onset of illness throughout ICU admission. There was no statistical significance between survivors and nonsurvivors using a modified t test. Day of illness data were collected via chart review. Horizontal bars represent mean±SEM. For each time point, there are the following number of samples: IFN- [symbol] survivors: 0, 8, 10, 10, 8, 4; IFN- [symbol] nonsurvivors: 0, 4, 7, 3, 3, 0; TNF-α survivors: 0, 8, 7, 9, 6, 3; TNF-α nonsurvivors: 0, 5, 5, 4, 3, 0.

FIG. 8. COVID-19 induces profound depletion of circulating immune effector cells. Absolute numbers of various white blood cell types (displayed as cells/μL) were determined in COVID-19-positive and CINS patients (red dots). ALC was determined by Barnes-Jewish Hospital Clinical Laboratory as part of patient clinical laboratory tests. CD3⁺ T, CD4⁺ T, CD8⁺ T, and NK cell and monocyte quantification was performed using flow cytometry as described in Methods. Pink shading represents normal reference values for healthy individuals at Barnes-Jewish Hospital Laboratories. Analysis by ANOVA with Dunnett's multiple comparison tests showed a significant decrease in ALC from CINS to COVID-19 days 1-3; P=0.01. ALC for CINS n=6; ALC for COVID-19 days 1-3 n=15, days 4-7 n=14, days 8-11 n=12, days 12-15 n=6. Cell counts for CD3⁺, CD4⁺, CD8⁺, and NK cells and monocytes: CINS n=6; COVID-19 days 1-3 n=15, days 4-7 n=14, days 8-11 n=10, days 12-15 n=4.

FIG. 9. IL-7 restores adaptive immune function in patients with COVID-19. Line plot demonstrating change in the number of cytokine-producing cells using ELISpot between control (anti-CD3/anti-CD28 antibody or LPS) samples and stimulation with IL-7 for IFN- [symbol] (A) and TNF-α (B). Each dot represents and individual patient. Red lines represent values for patients who died. IL-7 caused a significant increase in the number of IFN- [symbol] -producing T cells in COVID-19 patients; ****P<0.0001. IL-7 did not increase monocyte TNF-α production. (C and D) Representative photomicrographs demonstrating ELISpot change from control sample to IL-7 stimulated for IFN- [symbol] and TNF-α. Paired samples were analyzed using a paired Wilcoxon's rank-sum test. IFN- [symbol] n=25, TNF-α n=25.

FIG. 10. Individual patient IFN- [symbol] ELISpot production for healthy, CINS, septic and COVID-19. Bar graphs for individual patient immune response, represented as number of IFN- [symbol] cytokine producing T cells. (A) Healthy Control (n=27), (B) Critically ill non-septic (n=18). (C) Septic patients (n=46). (D) COVID-19 positive patients (n=25). Each bar represents an individual patient.

Red lines indicate patients who died. ELISpot assays were performed in duplicate for controls and triplicate for COVID-19 patients.

FIG. 11. Individual patient TNF-α ELISpot production for healthy, CINS, septic and COVID-19. Bar graphs for individual patient immune response, represented as number of TNF-α producing monocytes. (A) Healthy Controls (n=27), (B) Critically ill non-septic (n=18). (C) Septic patients (n=46). (D) COVID-19 positive patients (n=24). Each bar represents an individual patient. Red lines indicate patients who died. ELISpot assays were performed in duplicate for controls and triplicate for COVID-19 patients.

FIG. 12. IL-7 Effect on the innate and adaptive immune function in critically ill non-septic patients. Line plot demonstrating change in number of cytokine producing cells using ELISpot between control (anti-CD3/anti-CD28 antibody or LPS) samples and stimulation with IL-7 for IFN- [symbol] (A) and TNF-α (B). Panels (C), (D) are representative photomicrographs demonstrating ELISpot change from control sample to IL-7 stimulated for IFN- [symbol] and TNF-α. Paired samples were analyzed using a paired rank sum Wilcoxon test. IFN- [symbol] n=25, TNF-α n=25.

FIG. 13. IL-7 Effect on the innate and adaptive immune function in septic patients. Line plot demonstrating change in number of cytokine producing cells using ELISpot between control (anti-CD3/anti-CD28 antibody or LPS) samples and stimulation with IL-7 for IFN- [symbol] (A) and TNF-α (B). Panels (C), (D) are representative photomicrographs demonstrating ELISpot change from control sample to IL-7 stimulated for IFN- [symbol] and TNF-α. Each dot represents an individual patient. Red lines represent patients who died. Paired samples were analyzed using a paired rank sum Wilcoxon test. IFN- [symbol] n=25, TNF-α n=25.

FIG. 14. LUCID DURA flow tube gating strategy. Scatter plot for gating with FITC×SSC and histogram for singlets (counting beads1). All events that did not fall into the initial counting bead gate went through doublet discrimination (FSC-A×FSC-W, and then SSC-A×SSC-W) and were then examined for CD14 expression; these CD14+ cells were used to calculate number of monocytes. All events that were not CD14+ were then examined for CD56+ positivity (Used to calculate number of NK cells) and CD3 positivity (Used to calculate number of CD3+ T cells, CD56+CD3+ events were excluded). CD3+CD56− events were then further interrogated for CD4 or CD8 single positivity. Absolute cell counts were determined using the counting beads according to manufacturer instructions.

FIG. 15. PBMC gating strategy. Following doublet discrimination, PBMCs were gated for CD14 positivity. CD14+ cells were considered monocytes. All events not falling into the CD14+ gate were then examined on a FSC×SSC plot. Neutrophils and Lymphocytes were gated. Percentage of the whole singlet gate was then taken for use in determining cell number input in ELISpot assays.

FIG. 16. Unstimulated ex vivo production of IFN-γ and TNF-α in whole blood from septic patients using ELISpot assay. (A) Representative ELISpot images depicting IFN-γ production in media alone versus with CD3/CD28 Ab. (B) Graphic representation of n=15 septic patient responses between unstimulated and stimulated ex vivo cytokine production of IFN-γ. (C) Representative ELISpot images depicting TNF-α production in media alone versus with LPS. (D) Graphic representation of n=15 septic patient responses between unstimulated and stimulated ex vivo cytokine production of TNF-α. Red lines represent mortalities.

FIG. 17. Spot number and TWI of CD3/CD28-stimulated IFN-γ-producing cells using whole blood ELISpot assay. Graphic representation comparing the differential cytokine production, in terms of the number of activated cells, in healthy control (n=20), CINS (n=6), sepsis survivors (n=12), and sepsis nonsurvivors (n=7). The number of cells is represented as SFU, and the quantity of cytokine production is represented as TWI. (A) Whole blood IFN-γ production as SFU per microliter of blood. (B) IFN-γ production as SFU per 1000 lymphocytes. (C) TWI per microliter of whole blood. (D) TWI per 1000 lymphocytes. Each ELISpot assay was performed in duplicates. Bars represent mean±SEM. Group comparison using Kruskal-Wallis test with multiple comparisons corrected for FDR. Red dots represent mortalities. *p<0.05, **p<0.01.

FIG. 18. Spot number and TWI of LPS-stimulated TNF-α-producing cells using whole blood ELISpot assay. Graphic representation comparing the differential cytokine production, in terms of the number of activated cells, in healthy control (n=20), CINS (n=6), sepsis survivors (n=12), and sepsis nonsurvivors (n=7). The number of cells is represented as SFU, and the quantity of cytokine production is shown as TWI. (A) Whole blood TNF-α production as SFU per microliter of blood. (B) TNF-α production as SFU per 1000 lymphocytes plated. (C) TWI per microliter of blood. (D) TWI per 1000 lymphocytes. Each ELISpot assay is performed in duplicates. Bars represent mean±SEM. Group comparison using Kruskal-Wallis test with multiple comparisons corrected for FDR. Red dots represent mortalities. *p<0.05, **p<0.01, ***p<0.001, ****p<0.0001.

FIG. 19A-FIG. 19D. Comparison of T cell IFN-γ production in whole blood versus PBMCs ELISpot assay. (A) Representative figures depicting IFN-γ production of three individual patients using both whole blood and PBMC assays. (B-D) Dot plot graphs comparing data between whole blood and PBMC assays in healthy controls (n=20), sepsis survivors (n=12), and sepsis nonsurvivors (n=7). Colored dots represent individual patients for comparison between assays. Each ELISpot assay was performed in duplicates. Bars represent mean±SEM.

FIG. 20A-FIG. 20D. Comparison of myeloid cell TNF-α production in whole blood versus PBMCs ELISpot assay. (A) Representative figures depicting TNF-α production for three individual patients using both whole blood and PBMC assays. (B-D) Dot plot graphs compares data between whole blood and PBMC assays in healthy controls (n=20), sepsis survivors (n=12), and sepsis nonsurvivors (n=7). Colored dots represent individual patients used for comparison between assays. Each ELISpot assay was performed in duplicates. Bars represent mean±SEM.

FIG. 21A-FIG. 21D. TNF-α production in whole blood ELISpot assay following depletion of monocytes in healthy volunteers. (A) Graphic depiction representing TNF-α production in RBC-depleted blood versus RBC- and monocyte-depleted blood. (B) The number of monocytes plated per individual experiment (n=9) for RBC-depleted blood and RBC-plus monocyte-depleted blood. (C) The number of granulocytes/neutrophils plated per individual experiment (n=9) for RBC-depleted blood and RBC-plus monocyte-depleted blood. (D) The number of SFU for TNF-α production for RBC-depleted blood and RBC-plus monocyte-depleted blood. Statistical analysis using Wilcoxon ranked sum test. **p<0.01.

FIG. 22A-FIG. 22D. Adaptive immune function timeline for the first 7-10 d following diagnosis of sepsis. Representative images show the change in whole blood production of CD3/CD28-stimulated IFN-γ over time in patients with sepsis who survived (A) and those who did not survive (B). (C) IFN-γ production as SFU/μl comparing sepsis survivors (black line) to sepsis nonsurvivors (red line) on days 1-2, 3-5, and 6-10. (D) IFN-γ production as SFU per 1000 lymphocytes. Dots represent mean value, and bars represent ±SEM.

FIG. 23A-FIG. 23D. Innate immune function timeline for the first 7-10 d following diagnosis of sepsis. Images show the change in whole blood production of LPS-stimulated TNF-α over time in patients with sepsis who survived (A) and those who did not survive (B). (C) TNF-α production as SFU/μl comparing sepsis survivors (black line) to sepsis nonsurvivors (red line) on days 1-2, 3-5, and 6-10. (D) TNF-α production as SFU per 1000 myeloid cells. Dots represent mean value, and bars represent ±SEM.

FIG. 24. IFN-γ and TNF-α production in response to ex vivo IL-7 administration based on whole blood ELISpot assay in patients with sepsis. (A) Change in number of IFN-γ spots (SFU) in CD3/CD28 versus CD3/CD28+IL-7-stimulated cultures (n=19). (B) Representative ELISpot images showing the change in the number of IFN-γ spots in CD3/CD28 versus CD3/CD28+IL-7-stimulated cultures. (C) Change in the number of TNF-α spots (SFU) in LPS versus LPS+IL-7-stimulated cultures (n=19). (D) Representative ELISpot images showing the change in number of TNF-α spots in LPS versus LPS+IL-7-stimulated cultures. (A and C) The number of spots are the total number of spots per well and are not corrected for volume or cell number. Lines in red depict mortality. Statistical analysis performed using paired Wilcoxon ranked sum test. ***p<0.001.

FIG. 25. Gating strategy for cell typing by flow cytometry. (A) A preliminary size gate on a Forward×Side scatter plot was created to discern cells from debris. This gate was interrogated for CD14 positivity (Monocytes). A subsequent gate which excluded the monocytes (Boolean “not” gate) was utilized to determine lymphocyte and granulocyte population percentages on a Forward×Side scatter plot. Dot plots showing relative percentages of cell types forming the PBMC fraction in healthy controls (n=20), CINS (n=6), sepsis survivors (n=12) and sepsis non-survivors (n=7). (B) Monocyte fraction of PBMCs, (C) lymphocyte fraction of PBMCs and (D) neutrophil fraction of PBMCs. Bars represent mean+/−SEM. Statistical analysis performed with Kruskal Wallis test with multiple comparisons corrected using Dunn's test (*: p<0.05, **: p<0.01, ***: p<0.001). Red dots represent mortalities.

FIG. 26A-FIG. 26F. Comparison between surviving and non-surviving patients with sepsis in terms of severity scoring and comorbidities. There is no significant difference between sepsis survivors (n=12) vs. non-survivors (n=7) for sequential organ failure assessment (SOFA) score (A), Acute physiology and chronic health evaluation (APACHE) II score (B), or Charlson comorbidity score (C). (D, E, F) Dot plots of absolute leukocyte, lymphocyte and monocyte counts based on clinical labs on the day of initial blood draw for healthy control (n=20), CINS (n=6), sepsis survivors (n=12) and sepsis non-survivors (n=7). Bars represent mean+/−SEM. Group comparison using Kruskal Wallis test with multiple comparisons corrected for false discovery rate (*: p<0.05, **: p<0.01, ***: p<0.001, ****: p<0.0001).

FIG. 27A-FIG. 27F. IFN- [symbol] and TNF-α production in response to ex vivo IL-7 administration for whole blood and PBMC ELISpot in healthy controls (n=20), CINS (n=6) and septic patients (n=19). (A) Change IFN- [symbol] SFU to IL-7 in septic PBMC assay. (B) Change in TNF-α SFU to IL-7 for septic PBMC assay. (C) Change in IFN- [symbol] production in response to IL-7 for healthy controls. (D) Change in TNF-α production in response to IL-7 for healthy controls. (E) Change in IFN- [symbol] production in response to IL-7 for CINS. (F) Change in TNF-α production in response to IL-7 for CINS. The number of spots is reported as total number of spots per well and are not corrected for blood volume or cell number. Lines in red depict mortality. Statistical analysis performed using paired Wilcoxen ranked sum test (*: p<0.05, **: p<0.01, ***: p<0.001, ****: p<0.0001).

FIG. 28A-FIG. 28F. Serial clinical, anatomic, immunologic, and pathologic changes in an IL-7-treated patient with invasive fungal infection. A, Changes in WBC and ALC during the patient's hospital course. Days of hospitalization are indicated on the x-axis; IL-7 was initiated on day 59. Red arrows identify administration of IL-7. B, Color photographs of lower back and gluteal region demonstrating necrotic and infected areas; there was serial progression of the infection with necrotic regions, which resolved following initiation of IL-7. The green arrows in the second photo from the left show necrotic and poorly perfused margins. C, IL-7 mediates its effects to increase T cell IFN-γ production via STATS signaling. CyTOF demonstrated that there was a doubling of STATS expression in lymphocytes obtained after initiation of IL-7 compared with pretreatment; CyTOF data are reported at the geometric mean raw signal intensity for the gated population. All 3 samples were barcoded and run in a single experiment. The data presented are the geometric mean of the raw values for the gated populations (not normalized), as reported by the cytometer. D, Decreased T cell IFN-γ production is a hallmark of T cell exhaustion; ELISpot analysis on PBMCs were performed serially; the far-left ELISpot well had 47 IFN-γ-producing lymphocytes (depicted as SFU) per 1000 lymphocytes plated; the middle and far-right wells show a progressive increase in the both the number of lymphocytes in the PBMC fraction as well as an increase in the proportion of active IFN-γ-producing lymphocytes following CD3/CD28 stimulation. E, Immunohistochemical staining of wound margins: left panel: Gomori's methanamine silver stain highlighting invasive fungal hyphae (arrows), 400×; middle panel: hematoxylin and eosin stain showing necroinflammatory debris and refractile-appearing fungal hyphae (arrows), 400×; right panel: Gomori's methanamine silver stain highlighting shows clearance of invasive fungal microorganisms following IL-7 treatment; soft tissue necrosis is present, 400×. F, Immunohistochemical staining for CD3⁺ T cells. Note the marked increase in the number of lymphocytes in the wound margins that occurred after initiation of IL-7, 100×. Abbreviations: ALC, absolute lymphocyte count; CyTOF, time of flight mass cytometry; IFN-γ, interferon-γ; IL-7, interleukin-7; PBMCs, peripheral blood mononuclear cells; SFU, spot-forming units; WBC, white blood cell count.

FIG. 29. Time course of antimicrobial and surgical therapy. Graphical depiction of a timeline from the date of admission through the fourth month of his hospital course. This timeline demonstrates the surgical debridement occurrences, positive tissue cultures, antimicrobial therapies, and doses of recombinant human IL-7. Abbreviation: IL-7, interleukin-7.

FIG. 30A-FIG. 30F. Graphs demonstrating the trends in average daily vital signs throughout the course of IL-7 treatment beginning 3 days prior to the first dose of IL-7 and continues for 3 days following the last dose of IL-7. Vertical dotted lines represent the days in which IL-7 was administered. (A) Graph combining trends in heart rate, oxygen saturation, respiratory rate, temperature and mean arterial pressure. (B) Graph demonstrating increase in oxygen saturation from prior to IL-7 therapy and trends throughout this time. (C) Steady reduction in respiratory rate throughout the course of treatment. (D) Trends in heart rate. (E) Reduction in average temperature throughout IL-7 therapy. (F) Trends in mean arterial pressure.

FIG. 31A-FIG. 31E. Effect of dexamethasone on ex vivo interferon (IFN)-γ production in patients with coronavirus disease 2019 (COVID-19). Dot plots demonstrate dose decreased number of T cells secreting IFN-γ following CD3/CD28 stimulation after coincubation with increasing concentrations of dexamethasone phosphate, where (A), (B) represent effect of dexamethasone (n=11) and (C) represents the effects of dexamethasone together with interleukin (IL)-7. D and E, Representative enzyme-linked immunospot wells demonstrating a decreased number of CD3/CD28 stimulated IFN-γ-secreting cells when coincubated with increasing concentrations of dexamethasone. D, There is also a decrease in the total amount of cytokine produced per well as demonstrated by total well intensity (TWI), indicating a weaker overall response, and lower total cytokine production due to dexamethasone. E, Addition of IL-7 restores T cell function with increased number of IFN-γ-secreting cells with lower degree of suppression due to dexamethasone. Each spot in the representative images depicts an IFN-γ-secreting cell. SFU=spot forming units.

FIG. 32A-FIG. 32E. Effect of interleukin (IL)-7 on clinical and immunologic variables in critically ill coronavirus disease 2019 patient. A, A test dose of IL-7 (3 μg/kg) was administered followed by three additional doses of 10 μg/kg as indicated by red arrows. IL-7 increased the absolute lymphocyte count (ALC) from ˜400 lymphocytes/μL to over 5,000 lymphocytes/μL. Green lines indicate upper and lower limit of normal for ALC; IL-7 was well tolerated and did not show any evidence of worsening lung inflammation as indicated by observing a decreasing Fio₂ (B) and improving oxygen saturation (C). Blue arrows in (B) and (C) indicate day of intubation and extubation. Immunologic function of patient immune effector cells was tested by enzyme-linked immunospot (ELISpot). T cell interferon (IFN)-γ production was tested in peripheral blood mononuclear cells (PBMCs) stimulated with anti-CD3/CD28 before and after IL-7 therapy administration to the patient. 2.5×10⁴ PBMCs are plated for T cell functional assay. Note the dramatic increase in the number of lymphocytes that produce IFN-γ (as represented by number of spot forming units [SFUs]) in upper row after IL-7 therapy (D, top row); severe acute respiratory syndrome coronavirus 2 spike, and nucleocapsid peptide antigens were incubated overnight in patient PBMCs before and after IL-7 therapy. 1×10⁵ PBMCs are plated to assess antigen-specific T cells. Note the dramatic increase in the number of IFN-γ-producing lymphocytes (SFU) reacting specifically to the viral antigens after IL-7 (D, second row); innate immune function was tested by ELISpot assay for tumor necrosis factor (TNF)-α in PBMCs stimulated with lipopolysaccharide (LPS). 2.5×10³ PBMCs are plated to assess innate immune function. Note the marked increase in the number of TNF-α-producing cells (SFU) after IL-7 (E). CD=cluster of differentiation, Spo₂=oxygen saturation, NCAP=nucleocapsid protein.

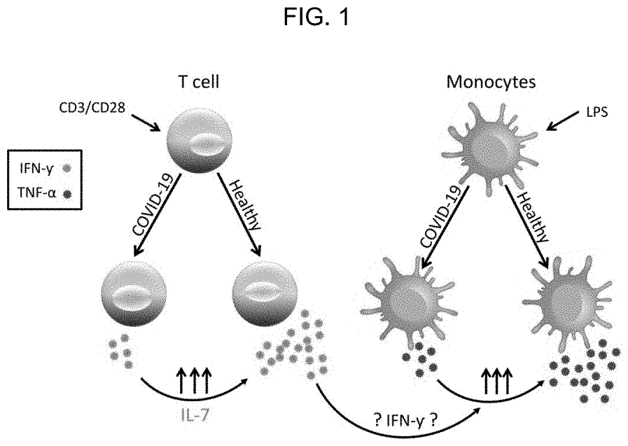

### DETAILED DESCRIPTION

Enzyme-linked immunosorbent spot (ELISpot) is a highly sensitive, functional immunoassay that measures the number of cytokine-secreting cells at the single-cell level in response to ex vivo stimulation. The ELISpot assay has excellent dynamic range and may detect as few as one in 100,000 cytokine-secreting cells. Furthermore, ELISpot can test simultaneously the integrity and robustness of the two disparate arms of immunity, i.e., innate (blood monocytes and low-density granulocytes) and adaptive cellular immunity (blood lymphocytes) by focusing on the responses of individual cell populations to cell-specific agonists.

The present disclosure show that the ELISpot assay can identify patients who are immune suppressed due to fungal sepsis or COVID and thus in need of drug therapies to boost their immune system. The present disclosure also shows that the ELISpot assay can be used to follow the response of the patient to different immune therapies and that the improved response indicated via the enhanced ELISpot results was associated with an improvement in the clinical status of the patients.

Here, it is shown that ELISpot assay can identify patients who are in the hyper-inflammatory phase of sepsis (thereby potentially needing drugs such as hydrocortisone to dampen the immune response) or, more commonly, patients who are severely immune suppressed thereby needing drugs to boost their immune system. ELISpot can be used to identify which immune enhancing drugs would be most likely to be beneficial in patients with sepsis who are immune suppressed.

Described herein is an improved method of ELISpot assay using diluted whole blood that determines the immune status of the patients, i.e., whether the patient is in a more hyper-inflammatory phase or an immunosuppressive phase. The disclosed ELISpot assay may also be useful in guiding drug therapy to improve the immune function of the patient. ELISpot wells coated with various drugs (e.g., tocilizumab, haptoglobin, hemopexin, ox40, IL7, steroids, and many more) have been evaluated using the presently disclosed platform. Because the immune status of COVID-19 patients might affect response to therapies, the use of immune-boosting and immune-suppressing therapies can be varied over the course of the disease.

The standard ELISpot assay is performed on peripheral blood mononuclear cells (PBMCs) isolated from the blood. The inability to use whole blood has been a significant limitation of the assay. Here is described a method for performing an ELISpot assay using a sample comprising whole blood or diluted whole blood to determine immune status.

As shown in Example 2, whole blood ELISpot was easy to perform, and results were generally comparable to PBMC-based ELISpot. However, the whole blood ELISpot assay revealed that nonmonocyte, myeloid populations are a significant source of ex vivo TNF-α production.

The present disclosure demonstrates a major advance in the ability to immune phenotype patients. The whole blood ELISpot assay is an effective method to quantify the functional state of patient adaptive and innate cellular function with excellent dynamic range. Circulating peripheral blood, which comprises RBCs, WBCs, platelets, cytokines, and chemokines, is considered in combination to be a vital functional organ. In this sense, it is highly informative to measure ex vivo cytokine production as a response to external stimuli in patient samples. Additionally, circulating chemokines and cytokines in the blood plasma fraction from patients with sepsis have potent immunologic effects on the function of the circulating WBCs, and removal through PBMC fractionation can dramatically change cellular response to stimuli or therapeutic molecules. Thus, as described herein, studies testing diluted whole blood are more likely to reflect the in vivo state. Reporting this response per volume of blood is fundamentally comparable between individual patients and offers more practical applicability than absolute cell counts. Finally, use of diluted whole blood has significant technical advantages of reduced preparation time and effort as well as avoiding potential biologic changes to fragile cells from patients because of Ficoll gradient separation or any other processing and handling of the sample.

In various aspects, the ELISpot assay may be used in combination with quantitation of circulating pro- and anti-inflammatory cytokines to classify a patient as exhibiting a hyper-inflammatory phase (an exaggerated pro-inflammatory “cytokine storm” or increase in cytokine production) or an immunosuppressive phase (reduction in immune cell production). As used herein, a hyper-inflammatory phase is characterized by an elevation in the production of cytokines including, but not limited to IL-1β, IFN- [symbol] , TNF-α, IL-6, and any combination thereof.

As described herein, an immunosuppressive phase is characterized by a reduction in T cell interferon-gamma (IFN- [symbol] ) production and monocyte TNF-α production obtained using the disclosed ELISpot assay. In some embodiments, the patients innate (blood monocytes and low-density granulocytes) and/or adaptive cellular immunity (blood lymphocytes) status may also be evaluated.

In some embodiments, those patients characterized as exhibiting a hyper-inflammatory phase may be treated by administering drugs to inhibit the host inflammatory response. In some embodiments, those patients characterized as exhibiting an immunosuppressive phase may be treated by administering an immuno-modulatory drug therapy or immune adjuvant that enhances host immunity including, but not limited to checkpoint inhibitors and common γ-chain cytokines, which stimulate CD4 and CD8 T cells, such as IL-17.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample. Other iterations of ELISpot, such as FluoroSpot can be used.

ELISpot/FluoroSpot

The assay as described herein, can comprise a capture antibody (e.g., anti-cytokine) attached, coated, or immobilized on a surface or membrane. An antigen-stimulated cell can secrete antigens that are captured by the capture antibody. The detection antibody can be added to bind the antigen captured on the capture antibody. A chromogen, a substance which can be readily converted into a dye or other colored compound, can be added.

An enzyme-linked immune absorbent spot (ELISpot) is a type of assay that focuses on quantitatively measuring the frequency of cytokine secretion for a single cell or a population of cells. The ELISpot Assay is also a form of immunostaining because it is classified as a technique that uses antibodies to detect an analyte or protein, such as a biological or chemical substance being identified or measured. The FluoroSpot Assay is a variation of the ELISpot assay. The FluoroSpot Assay uses fluorescence in order to analyze multiple analytes, thus it can detect the secretion of more than one type of analyte or protein.

Mechanism of ELISpot

Antibody coating (e.g., attached, immobilized, or coated): Throughout the ELISpot Assay technique, different substances are added to and washed away from wells. Wells are found on a laboratory plate with tiny dishes/bowls that can be filled with a substance to be examined; the amount of wells on a plate varies, but generally ranges from 16-100. The first substance added to the wells can be cytokine specific monoclonal antibodies. These antibodies can coat the walls of the wells for future binding to cytokine. The monoclonal antibodies are antibodies produced from a single cell lineage, and is only able to bind to one protein epitope. Polyclonal antibodies, on the other hand, are capable of binding to multiple epitopes of the same protein.

Cell incubation: The desired cells being observed and analyzed are added to the wells. Each well can have the presence or absence of stimuli that activate the secretion of cytokine in cells. During cell incubation, the cells are allowed to react to any present stimuli and secrete cytokine. Many procedures and methods are known in the art to follow to ensure proper cell handling. To make sure that cells are of high quality, cells in blood samples can be lightly agitated if stored for longer than 3 hours, the blood samples can be diluted in PBS (phosphate buffered saline) before being stored, and the blood samples may be free of granulocytes. Any cells that have been cryopreserved and thawed can be allowed to rest for an hour or more at 37° C. (the typical temperature of the human body). When incubating cells, some considerations can include, ensuring that the cells do not experience sudden movements that could affect spot formation or ensuring the incubator's humidity is high enough to avoid excessive evaporation and drying out the wells.

Cytokine capture: Since the cells are surrounded by cytokine-specific monoclonal antibodies that coat the walls of the wells, a cytokine that has been secreted by the incubated cells will start to attach to the antibodies at a specific epitope.

Detection antibodies: At this point, the wells can be rinsed in order to get rid of the cells and any other undesirable substances. Remaining are the cytokine specific monoclonal antibodies and any cytokine that bonded to the antibodies. Biotinylated cytokine-specific detection antibodies can then be added to the well. These cytokine-specific detection antibodies will bind to any cytokine that is left in the well since the cytokine is still attached to the first set of antibodies used. Because the cytokine is attached to the first set of antibodies coating the wells, the cytokine is not washed away when the wells are rinsed.

Streptavidin-enzyme conjugate: Streptavidin-enzyme conjugate can be added to the wells in order to bind with the detection antibodies. The purpose of biotinylating the cytokine-specific detection antibodies added to the wells in the previous step is so that the antibody can bind to the new streptavidin-enzyme conjugate. Biotinylation creates a strong affinity between the biotin on the cytokine-specific antibody and the streptavidin on the conjugate.

Addition of substrate: A substrate (e.g., a chromogenic substrate) can be added to the wells, and is catalyzed by the enzyme conjugate added in the previous step. This reaction forms insoluble precipitate that forms spots in the wells. The substrate that is used in this step can depend on the type of enzyme used in the previous step. If streptavidin-ALP (streptavidin and alkaline phosphatase conjugate) is used, then using BCIP/NBT-plus (a mixture of 5-bromo-4-chloro-3-indolyl phosphate and nitroblue tetrazolium chloride) as a substrate can produce more distinct spots that are easier to analyze. If streptavidin-HRP (streptavidin and horseradish peroxidase conjugate) is used, then using TMB (tetramethylbenzidine) as a substrate can produce better results.

Analysis: The spots that are formed can then be read on an automated ELISpot reader, or counted under a dissection microscope, and further used to calculate the frequency of cytokine secretion.

Mechanism of FluoroSpot

The FluoroSpot assay combines the sensitivity of ELISpot with the capacity to study secretion of several analytes simultaneously, enabling studies of cell populations with different functional profiles. The FluoroSpot assay is very similar to the ELISpot assay. The key difference is that the FluoroSpot assay can analyze the presence of multiple analytes on one plate of wells, whereas the conventional ELISpot assay can only analyze one analyte at a time. The FluoroSpot assay accomplishes this by using fluorescence rather than an enzymatic reaction for detection. The steps for a FluoroSpot assay are also similar, with a few differences.

Antibody Coating: Similar to the ELISpot, cytokine-specific monoclonal capture antibodies are added to a plate with wells. For both assays, the plates are ethanol-treated to avoid contamination and skewed data collection. For the FluoroSpot assay, a mixture of different types of capture antibodies are attached to the wells in order to detect multiple types of analytes. In order to get optimal results with the ELISpot and the FluoroSpot assay, proper plate coating techniques should be followed. The plates should be treated with ethanol, washed, and then coated with antibodies. Ethanol treatment methods also vary depending on the type of plates that are used. For MSIP and IPFL plates, one can add 15 micro liters of 35% ethanol to all of the wells. Allow the ethanol to sit in the wells for one minute, and then pour it out. For MAIPSWU plates, one can instead add 50 micro liters of 70% ethanol to all of the wells. Allow the ethanol to sit in the wells for two minutes, and then pour it out. After the wells are treated with ethanol, the wells can be washed with about 200 micro liters of sterile water. This washing process can be repeated a total of 5 times. Once the wells have been treated with ethanol and washed, the cytokine-specific monoclonal capture antibodies can be added to each well.

Cell Incubation: a cell or population of cells can be added to the wells and incubated in the presence or absence of stimuli that affect protein secretion.

Cytokine Capture: Proteins/analytes that are secreted by the incubated cells will bind to the capture antibodies attached, immobilized, or coated to the wells during the first step.

Detection Antibodies: Similar to the ELISpot, once the wells are rinsed to remove cells and other substances that are not of interest for identifying or measuring, a biotinylated detection antibody can be added (this can be specific for one type of analyte that will be quantified) and then tag-labeled detection antibodies are added for optional second or third types of analytes being studied.

Fluorophore-labeled Conjugates: Instead of adding a streptavidin-enzyme conjugate, the detection of multiple analytes is amplified in the FluoroSpot with the use of fluorophore-labeled anti-tag antibody and streptavidin-fluorophore conjugate. A fluorescence enhancer solution can also be added during this step in order to enhance the signals later used when analyzing the fluorescence colors in the wells. This fluorescence makes it possible for the FluoroSpot to analyze and compare multiple analytes, unlike the ELISpot.

Analysis: Because FluoroSpot relies on the use of fluorescence and not an enzymatic reaction, there is no need for a step that adds a substrate to react with enzymes (as needed for the ELISpot). The last step for the FluoroSpot assay is to analyze the fluorophores under an automated fluorescence reader that has separate filters for the different fluorophores being analyzed. These filters can be selected for the specific wavelengths of the fluorophores for accurate measurements.

Since the FluoroSpot assay identifies and quantifies the presence of multiple analytes, it is possible that the absorption of one analyte can affect the secretion of another analyte; this is called capture effects. The affect an analyte (e.g., cytokine) has on another analyte could be positive or negative (the production of the second analyte can either increase or decrease). To counteract capture effects, it is possible to use co-stimulation in order to bypass the decreased production of an analyte. This is when a second antibody that stimulates the production of the same analyte is added to the wells.

The ELISpot and FluoroSpot assays can be used in many research fields: vaccine development, cancer, allergies, monocytes/macrophages/dendritic cells characterization, apolipoproteins analysis, and veterinary research. With the ELISpot, antigen-specific cytokine responses, antibody specific secreting cells, tumor antigens, granzyme B and Perforin release by T cells, vaccine efficacy, epitope mapping, cytotoxic T cell activity, detection of IL-4, IL-5, and IL-13, vaccine-induced antibody responses, antigen-specific memory B cells, and more can be studied.

As an example, the T cell ELISpot assay can be used to characterize T cell subsets. This is because the assay can detect the production of cytokines IFN-y, IL-2, TNF-alpha, IL-4, IL-5, and IL-13. The first three cytokines are produced by Th1 cells, while the last three are produced by Th2 cells. Measuring T cell responses through cytokine production can also make it possible to study vaccine efficacy. As another example, with T cell FluoroSpot, tumor-infiltrating lymphocytes can be monitored. The IFN-y cytokine and granzyme B secretion can be analyzed in order to assess cytotoxic T cell responses. Both of these are used for cancer research. As yet another example, with B-cell FluoroSpot, vaccine efficacy can also be observed by quantifying the secretion of IgG, IgA, and IgM before and after vaccination. This analysis of multiple immunoglobulins is made possible because of the fluorescence method used in the FluoroSpot.

Biological Samples

A biological sample can comprise or be whole blood, diluted whole blood, peripheral blood, or isolated human peripheral blood mononuclear cell (PBMC). PBMCs are a diverse mixture of highly specialized immune cells that play key roles in keeping our bodies healthy. A peripheral blood mononuclear cell (PBMC) is any blood cell having a round nucleus such as lymphocyte (e.g., T cells, B cells), monocyte, or a macrophage. These blood cells are a critical component in the immune system to fight infection and adapt to intruders. Two primary techniques that separate peripheral blood mononuclear cells from whole peripheral blood are through the use of a density gradient centrifugation process or by leukapheresis. Blood contains many types of cells: white blood cells (monocytes, lymphocytes, neutrophils, eosinophils, basophils, and macrophages), red blood cells (erythrocytes), and platelets. Peripheral blood cells are the cellular components of blood, comprising red blood cells (erythrocytes), white blood cells (leucocytes), and platelets, which are found within the circulating pool of blood and not sequestered within the lymphatic system, spleen, liver, or bone marrow.

Disclosed herein is the use of a biological sample comprising whole blood, which does not require the isolation of PBMCs. Whole blood ELISpot can test simultaneously the integrity and robustness of the two disparate arms of immunity, i.e., innate (blood monocytes and low-density granulocytes) and adaptive cellular immunity (blood lymphocytes) by focusing on the responses of individual cell populations to cell-specific agonists. As shown in Example 1, T cell subsets were profoundly reduced in COVID-19 patients. Additionally, stimulated blood mononuclear cells produced less than 40%-50% of the IFN- [symbol] and TNF-α observed in septic and CINS patients, consistent with markedly impaired immune effector cell function.

Cytokines/Chemokines

As described herein, secreted cytokines are measured to determine immune status of a subject. As an example, the cytokines are measured after ex vivo stimulation. Cytokines that can be measured to determine the immune status (cytokines associated with cellular immunity) of a subject can be human cytokine such as IL-1β, IFN- [symbol] , TNF-α, IL-6, or those listed below associated with cellular immunity. Inflammatory cytokines can include interleukin-1 (IL-1), IL-12, and IL-18, tumor necrosis factor alpha (TNF-α), interferon gamma (IFNγ), and granulocyte-macrophage colony stimulating factor (GM-CSF). An anti-inflammatory cytokine can be interleukin (IL)-1 receptor antagonist, IL-4, IL-6, IL-10, IL-11, and IL-13.

| | | | | | Cytokine | Receptor |
|---|---|---|---|---|---|---|
| | | | Chromosome | Molecular | Receptor(s) | Location |
| Name | Synonym(s) | Amino | Acids | Weight² | (Da) and Form | (s) |
| Interleukins | | | | | | |
| IL-1-like | | | | | | |
| IL-1α | hematopoietin-1 | 271 | 2q14 | 30606 | CD121a, | 2q12, |
| | | | | | CDw121b | 2q12-q22 |
| IL-1β | catabolin | 269 | 2q14 | 20747 | CD121a, | 2q12, |
| | | | | | CDw121b | 2q12-q22 |
| IL-1RA | IL-1 receptor | 177 | 2q14.2 | 20055 | CD121a | 2q12 |
| | antagonist | | | | | |
| IL-18 | interferon-γ | 193 | 11q22.2- | 22326 | IL- | 2q12 |
| | inducing | | q22.3 | | 18Rα, β | |
| | factor | | | | | |
| Common g | | | | | | |
| chain (CD132) | | | | | | |
| IL-2 | T cell growth | 153 | 4q26- | 17628 | CD25, | 10p15- |
| | factor | | q27 | | 122, | p14, |
| | | | | | 132 | 22q13.1, |
| | | | | | | Xq13.1 |
| IL-4 | BSF-1 | 153 | 5q31.1 | 17492 | CD124, | 16p11.2- |
| | | | | | 213a13, | 12.1, X, |
| | | | | | 132 | Xq13.1 |
| IL-7 | | 177 | 8q12- | 20186 | CD127, | 5p13, |
| | | | q13 | | 132 | Xq13.1 |
| IL-9 | T cell growth | 144 | 5q31.1 | 15909 | IL-9R, | Xq28 or |
| | factor P40 | | | | CD132 | Yq12, |
| | | | | | | Xq13.1 |
| IL-13 | P600 | 132 | 5q31.1 | 14319 | CD213a1, | X, Xq13.1- |
| | | | | | 213a2, | q28, |
| | | | | | CD1243, | 16p11.2- |
| | | | | | 132 | 12.1, |
| | | | | | | Xq13.1 |
| IL-15 | | 162 | 4q31 | 18086 | IL-15Ra, | 10p14- |
| | | | | | CD122, | p14, |
| | | | | | 132 | 22q13.1, |
| | | | | | | Xq13.1 |
| Common b | | | | | | |
| chain (CD131) | | | | | | |
| IL-3 | multipotential | 152 | 5q31.1 | 17233 | CD123, | Xp22.3 or |
| | CSF, MCGF | | | | CDw131 | Yp11.3, |
| | | | | | | 22q13.1 |
| IL-5 | BCDF-1 | 134 | 5q31.1 | 15238, | CDw125, | 3p26-p24, |
| | | | | homodimer | 131 | 22q13.1 |
| Also related | | | | | | |
| GM-CSF | CSF-2 | 144 | 5q31.1 | 16295 | CD116, | Xp22.32 or |
| | | | | | CDw131 | Yp11.2, |
| | | | | | | 22q13.1 |
| IL-6-like | | | | | | |
| IL-6 | IPN-β2, | 212 | 7p21 | 23718 | CD126, | 1q21, |
| | BSF-2 | | | | 130 | 5q11 |
| IL-11 | AGIF | 199 | 19q13.3- | 21429 | IL-11Ra, | 9p13, |
| | | | 13.4 | | CD130 | 5q11 |
| Also related | | | | | | |
| G-CSF | CSF-3 | 207 | 17q11.2- | 21781 | CD114 | 1p35- |
| | | | q12 | | | p34.3 |
| IL-12 | NK cell | 219/328 | 3p12- | 24844/37169 | CD212 | 19p13.1, |
| | stimulatory | | p13.2/ | | | 1p31.2 |
| | factor | | | | | |
| | | | 5q31.1- | heterodimer | | |
| | | | q33.1 | | | |
| LIF | leukemia | 202 | 22q12.1- | 22008 | LIFR, | 5p13-p12 |
| | inhibitory | | q12.2 | | CD130 | |
| | factor | | | | | |
| OSM | oncostatin M | 252 | 22q12.1- | 28484 | OSMR, | 5p15.2- |
| | | | q12.2 | | CD130 | 5p12 |
| IL-10-like | | | | | | |
| IL-10 | CSIF | 178 | 1q31- | 20517, | CDw210 | 11q23 |
| | | | q32 | homodimer | | |
| IL-20 | | 176 | 2q32.2 | 20437 | IL-20Rα, β | ? |
| Others | | | | | | |
| IL-14 | HMW-BCGF | 498 | 1 | 54759 | IL-14R | ? |
| IL-16 | LCF | 631 | 15q24 | 66694, | CD4 | 12pter- |
| | | | | homotetramer | | p12 |
| IL-17 | CTLA-8 | 155 | 2q31 | 17504, | CDw217 | 22q11.1 |
| | | | | homodimer | | |
| Interferons | | | | | | |
| IFN-α | | 189 | 9p22 | 21781 | CD118 | 21q22.11 |
| IFN-β | | 187 | 9p21 | 22294 | CD118 | 21q22.11 |
| IFN-γ | | 166 | 12q14 | 19348, | CDw119 | 6q23- |
| | | | | homodimer | | q24 |
| TNF | | | | | | |
| CD154 | CD40L, | 261 | Xq26 | 29273, | CD40 | 20q12- |
| | TRAP | | | homotrimer | | q13.2 |
| LT-β | | 244 | 6p21.3 | 25390, | LTβR | 12p13 |
| | | | | heterotrimer | | |
| TNF-α | cachectin | 233 | 6p21.3 | 25644, | CD120a, b | 12p13.2, |
| | | | | homotrimer | | 1p36.3- |
| | | | | | | p36.2 |
| TNF-β | LT-α | 205 | 6p21.3 | 22297, | CD120a, b | 12p13.2, |
| | | | | heterotrimer | | 1p36.3- |
| | | | | | | p36.2 |
| 4-1BBL | | 254 | 19p13.3 | 26624, | CDw137 | 1p36 |
| | | | | trimer? | (4-1BB) | |
| APRIL | TALL-2 | 250 | 17p13.1 | 27433, | BCMA, TACI | 16p13.1, |
| | | | | trimer? | | 17p11.2 |
| CD70 | CD27L | 193 | 19p13 | 21146, | CD27 | 12p13 |
| | | | | trimer? | | |
| CD153 | CD30L | 234 | 9q33 | 26017, | CD30 | 1p36 |
| | | | | trimer? | | |
| CD178 | FasL | 281 | 1q23 | 31485, | CD95 (Fas) | 10q24.1 |
| | | | | trimer? | | |
| GITRL | | 177 | 1q23 | 20307, | GITR | 1p36.3 |
| | | | | trimer? | | |
| LIGHT | | 240 | 16p11.2 | 26351, | LTbR, HVEM | 12p13, |
| | | | | trimer? | | 1p36.3- |
| | | | | | | p36.2 |
| OX40L | | 183 | 1q25 | 21050, | OX40 | 1p36 |
| | | | | trimer? | | |
| TALL-1 | | 285 | 13q32-q34 | 31222, | BCMA, | 16p13.1, |
| | | | | trimer? | TACI | 17p11.2 |
| TRAIL | Apo2L | 281 | 3q26 | 32509, | TRAILR1-4 | 8p21 |
| | | | | trimer? | | |
| TWEAK | Apo3L | 249 | 17p13.3 | 27216, | Apo3 | 1p36.2 |
| | | | | trimer? | | |
| TRANCE | OPGL | 317 | 13q14 | 35478, | RANK, OPG | 18q22.1, |
| | | | | trimer? | | 8q24 |
| TGF-β | | | | | | |
| TGF-β1 | TGF-β | 390 | 19q13.1 | 44341, | TGF-βR1 | 9q22 |
| | | | | homodimer | | |
| TGF-β2 | | 414 | 1q41 | 47747, | TGF-βR2 | 3p22 |
| | | | | homodimer | | |
| TGF-β3 | | 412 | 14q24 | 47328, | TGF-βR3 | 1p33- |
| | | | | homodimer | | p32 |
| Miscellaneous | | | | | | |
| hematopoietins | | | | | | |
| Epo | erythropoietin | 193 | 7q21 | 21306 | EpoR | 19p13.3- |
| | | | | | | p13.2 |
| Tpo | MGDF | 353 | 3q26.3- | 37822 | TpoR | 1p34 |
| | | | q27 | | | |
| Flt-3L | | 235 | 19q13.1 | 26416 | Flt-3 | 13q12 |
| SCF | stem cell | 273 | 12q22 | 30898, | CD117 | 4q11- |
| | factor, c-kit | | | homodimer | | q12 |
| | ligand | | | | | |
| M-CSF | CSF-1 | 554 | 1p21- | 60119, | CD115 | 5q33- |
| | | | p13 | homodimer | | q35 |
| MSP | Macrophage | 711 | 3p21 | 80379 | CDw136 | 3p21.3 |
| | stimulating | | | | | |
| | factor, MST-1 | | | | | |

¹List assembled using data from Gene Cards (World Wide Web URL: http://genome-www.stanford.edu/genecards). Note that some of the cytokines listed are not discussed in this chapter.
²Data describes the unprocessed precursor.
³Can be found in complexes.

Chemokines as described herein can be any of those listed below.

| | | Amino | Ligand | Molecular | Chemokine | Receptor |
|---|---|---|---|---|---|---|
| Name | Synonym(s) | Acids² | Location | Weight (Da)² | Receptor(s) | Location |
| C Chemokines | | | | | | |
| XCL1 | lymphoactin a, | 114 | 1q21-q25 | 12517 | XCR1 | 3p21 |
| | SCM-1a, ATAC | | | | | |
| XCL2 | lymphoactin b, | 114 | 1q23 | 12567 | XCR1 | 3p21 |
| | SCM-1b, ATAC | | | | | |
| CC Chemokines | | | | | | |
| CCL1 | I-309 | 96 | 17 | 10992 | CCR8 | 3p22 |
| CCL2 | MCP-1, | 99 | 17q11.2- | 11025 | CCR2 | 3p21 |
| | MCAF | | q12 | | | |
| CCL3 | MIP-1α, | 92 | 17q11- | 10085 | CCR1, CCR5 | 3p21 |
| | LD78α | | q21 | | | |
| CCL4 | MIP-1β, | 92 | 17q11- | 10212 | CCR5 | 3p21 |
| | LAG-1, | | q23 | | | |
| | ACT-2 | | | | | |
| CCL5 | RANTES | 91 | 17q11.2- | 9990 | CCR1, CCR3, | 3p21 |
| | | | q12 | | CCR5 | |
| CCL7 | MCP-3 | 99 | 17q11.2- | 11200 | CCR1, CCR2, | 3p21 |
| | | | q12 | | CCR3 | |
| CCL8 | MCP-2 | 99 | 17q11.2 | 11246 | CCR3 | 3p21 |
| CCL11 | eotaxin | 97 | 17q21.1- | 10732 | CCR3 | 3p21 |
| | | | q21.2 | | | |
| CCL13 | MCP-4 | 98 | 17q11.2 | 10986 | CCR2, CCR3 | 3p21 |
| CCL14 | HCC-1 | 93 | 17q11.2 | 10678 | CCR1 | 3p21 |
| CCL15 | HCC-2, Lkn-1, | 113 | 17q11.2 | 12248 | CCR1, CCR3 | 3p21 |
| | MIP-1d, MIP-5 | | | | | |
| CCL16 | HCC-4, LEC, | 120 | 17q11.2 | 13600 | CCR1 | 3p21 |
| | LMC, LCC-1 | | | | | |
| CCL17 | TARC | 94 | 16q13 | 10507 | CCR4 | 3p24 |
| CCL18 | DC-CK1, PARC, | 89 | 17q11.2 | 9849 | ? | |
| | AMAC-a, MIP-4 | | | | | |
| CCL19 | MIP-3β, ELC, | 98 | 9p13 | 10993 | CCR7 | 17q12- |
| | exodus-3 | | | | | q21.1 |
| CCL20 | MIP-3α, LARC, | 96 | 2q33-q37 | 10762 | CCR6 | 6q27 |
| | exodus-1 | | | | | |
| CCL21 | 6Ckine, SLC, | 134 | 9p13 | 14646 | CCR7 | 17q12- |
| | exodus-2 | | | | | q21.2 |
| CCL22 | MDC, STCP-1 | 93 | 16q13 | 10580 | CCR4 | 3p22 |
| CCL23 | MPIF-1, MIP-3, | 120 | 17q11.2 | 13443 | CCR1 | 3p21 |
| | CKb-8 | | | | | |
| CCL24 | MPIF-2, eotaxin- | 119 | 7q11.23 | 13133 | CCR3 | 3p21 |
| | 2, CKb-6 | | | | | |
| CCL25 | TECK, MIP-4a | 150 | 19p13.2 | 16639 | CCR9 | 3p21 |
| CCL26 | eotaxin-3 | 94 | 7q11.2 | 10648 | CCR3 | 3p21 |
| CCL27 | Eskine, CTACK, | 112 | 9p13 | 12618 | CCR10 | 3p21 |
| | ILC | | | | | |
| CXC Chemokines | ELR? | | | | | |
| CXCL1 | GROa, MGSA-a+ | 107 | 4q21 | 11301 | CXCR1, | 2q35 |
| | | | | | CXCR2 | |
| CXCL2 | GROb, MGSA-b, | 107 | 4q21 | 11389 | CXCR2 | 2q35 |
| | MIP-2a+ | | | | | |
| CXCL3 | GROg, MGSA-g, | 107 | 4q21 | 11342 | CXCR2 | 2q35 |
| | MIP-2b+ | | | | | |
| CXCL4 | PF4, oncostatin | 101 | 4q12-q13 | 10845 | ? | |
| | A− | | | | | |
| CXCL5 | ENA-78+ | 114 | 4q13-q21 | 11972 | CXCR2 | 2q35 |
| CXCL6 | GCP-2+ | 114 | 4q21 | 11897 | CXCR1, | 2q35 |
| | | | | | CXCR2 | |
| CXCL7 | NAP-2, PPBP+ | 375 | 4q12-13 | 42823 | CXCR2 | 2q35 |
| CXCL8 | IL-8, NAP-1, | 99 | 4q12-13 | 11098 | CXCR1, | 2q35 |
| | NAF, MDNCF+ | | | | CXCR2 | |
| CXCL9 | Mig− | 125 | 4q21 | 14019 | CXCR3 | Xq13 |
| CXCL10 | IP-10− | 98 | 4q21 | 10856 | CXCR3 | Xq13 |
| CXCL11 | I-TAC− | 94 | 4q21.2 | 10365 | CXCR3 | Xq13 |
| CXCL12 | SDF-1α/β− | 93 | 10q11.1 | 10666 | CXCR4 | 2q21 |
| CXCL13 | BLC, BCA-1− | 109 | 4q21 | 12664 | CXCR5 | 11 |
| CXCL14 | BRAK− | 99 | 5q31 | 11722 | ? | |
| CX3C Chemokines | | | | | | |
| CX3CL1 | fractalkine | 397 | 16q13 | 42202 | CX3CR1 | 3p21 |

¹List based on terminology from Zlotnick and Yoshie, 2000 with added annotation and data from Gene Cards (World Wide Web URL: http://genome-www.stanford.edu/genecards).
²Data describes the unprocessed precursor.

Screening

Also provided are screening methods for potential therapeutics or test therapeutic agents. Potential therapeutics can be added to the wells or coated on the wells. Agents that can be coated on the wells or added to the wells can be biological molecules (e.g., IL-7), small molecules (e.g., steroids), or inorganic small molecules or cytokines/chemokines, IL-6, Pd1, anti-PD1, IL-7, IL-10, Ox40, etc. The described assays can reveal potential immune adjuvant therapies that might effectively reverse immunosuppression. There are several immune adjuvants that are undergoing clinical trials in sepsis (e.g., anti-PD-1, anti-PD-L1, GM-CSF, and IL-7). In the present disclosure, IL-7 added ex vivo to septic patient samples effectively restored T cell IFN-γ production in the majority of septic patients. By restoring host immunity, IL-7 could potentially accelerate the eradication of the primary infection and decrease secondary hospital-acquired infections. Previously, our group reported that anti-PD-1, anti-PD-L1, and OX-40 agonistic Abs are also effective in restoring T cell IFN-γ production in a variable percentage of septic patients using a PBMC ELISpot assay (see e.g., Thampy et al. Restoration of T cell function in multi-drug resistant bacterial sepsis after interleukin-7, anti-PD-L1, and OX-40 administration. PLoS One. 2018; 13(6):e0199497). Thus, the ELISpot assay could be used to identify the optimal immune therapy for use in individual septic patients. This method undoubtedly holds translatable potential to many other fields within critical care, oncology, and autoimmune disease.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to wells coated with potential therapeutics, such as biological molecules (e.g., IL-7), small molecules (e.g., steroids), inorganic small molecules, cytokines/chemokines (IL6, etc.), PD1, anti-PD1, IL-7, IL-10, Ox40, etc. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Severe Immunosuppression and Nota Cytokine Storm Characterizes COVID-19 Infections

Abstract

COVID-19-associated morbidity and mortality have been attributed to a pathologic host response. Two divergent hypotheses have been proposed: hyper-inflammatory cytokine storm; and failure of host protective immunity that results in unrestrained viral dissemination and organ injury. A key explanation for the inability to address this controversy has been the lack of diagnostic tools to evaluate immune function in COVID-19 infections. ELISpot, a highly sensitive, functional immunoassay, was employed in 27 patients with COVID-19, 51 patients with sepsis, 18 critically ill nonseptic (CINS) patients, and 27 healthy control volunteers to evaluate adaptive and innate immune status by quantitating T cell IFN- [symbol] and monocyte TNF-α production. Circulating T cell subsets were profoundly reduced in COVID-19 patients. Additionally, stimulated blood mononuclear cells produced less than 40%-50% of the IFN- [symbol] and TNF-α observed in septic and CINS patients, consistent with markedly impaired immune effector cell function. Approximately 25% of COVID-19 patients had increased IL-6 levels that were not associated with elevations in other canonical proinflammatory cytokines. Collectively, these findings support the hypothesis that COVID-19 suppresses host functional adaptive and innate immunity. Importantly, IL-7 administered ex vivo restored T cell IFN- [symbol] production in COVID-19 patients. Thus, ELISpot can functionally characterize host immunity in COVID-19 and inform prospective therapies.

### Definitions

| Abbreviation | Explanation |
|---|---|
| PNA | Pneumonia |
| UTI | Urinary Tract Infection |
| MRSA | Methicillin-Resistant Staphylococcus Aureus |
| MSSA | Methicillin-Sensitive Staphylococcus Aureus |
| VAP | Ventilator-Associated Pneumonia |
| COVID-19 | 2019 Novel Coronavirus |
| CAP | Community-Acquired Pneumonia |
| ARDS | Acute Respiratory Distress Syndrome |
| SIRS | Systemic Inflammatory Response Syndrome |
| ARF | Acute Respiratory Failure |
| AHRF | Acute Hypoxemic Respiratory Failure |
| MVC | Motor Vehicle Crash |

### INTRODUCTION

One of the most remarkable realities about the current SARS-CoV-2 infection outbreak (COVID-19) is that despite intense worldwide investigations, the decisive pathophysiologic processes that are responsible for patient morbidity and mortality remain unknown. Currently, the predominant paradigm is that an overexuberant immune response mediated by excessive proinflammatory cytokines drives excessive lung injury and a procoagulant state (1-7). Accordingly, death is assumed to be primarily due to inflammatory lung injury, disturbances in micro- and macrocirculation, and resultant respiratory failure or vascular coagulopathy (8-14). This concept of a cytokine storm-mediated death in COVID-19 patients has been popularized in both the lay press and many leading scientific publications (6, 15). Based on this theory, a number of anti-cytokine and anti-inflammatory therapies are being tested in COVID-19, including anti-IL-6(R) antibodies, IL-1 receptor antagonists, and JAK/STAT inhibitors, with early trial results failing to demonstrate significant efficacy (2, 3, 9, 15-18).

Paradoxically, a second and diametrically opposed theory for COVID-19-induced morbidity and mortality is an “immunologic collapse” of the host's protective system (15, 19-21). This collapse of host protective immunity manifests itself as a failure to control unrestrained viral replication and dissemination with direct host cytotoxicity. Support for this contrasting theory is based on the observed progressive and profound lymphopenia, often to levels seen in patients with AIDS (22). Multiple recent studies show that unlike the cytokine storm, which is often considered episodic, lymphopenia is incessant in critically ill COVID-19 patients with and correlates with increased secondary infections and death (11, 13). Postmortem studies of patients dying of COVID-19 have also described a devastating loss of immune cells in spleen and secondary lymphoid organs (23). Multiple lymphocyte subsets are lost, including CD4⁺ T, CD8⁺ T, and NK cells, which play vital antiviral roles, and in B cells, which are essential for making antibodies that neutralize the virus (4, 21, 24-26).

Personalized medicine approaches require a better understanding of which of these immune endotypes predominate, because the appropriate intervention is diametrically different depending upon whether the patient is experiencing hyper-inflammation or profound immunosuppression. For example, anti-IL-6(R) antibodies, IL-1 receptor antagonists, and JAK/STAT inhibitors are currently undergoing clinical testing in patients with COVID-19 (27-32) and carry the potential to further compromise the patient's ability to eradicate the virus. Conversely, treatment with immune stimulants such as checkpoint inhibitors, IL-7, IFN-γ, and GM-CSF, currently either proposed or in active clinical trials in COVID-19 (15, 33), could exacerbate a dysfunctional and robust inflammatory response and worsen organ injury.

Two distinct and key questions must be addressed in critically ill COVID-19 patients: (i) what is their primary immune endotype, i.e., hyper-inflammatory versus immunosuppressive? and (ii) how does each evolve over time with regard to disease progression or resolution. A better understanding of the COVID-19 patient's immune status would be instrumental in guiding proper immunotherapy.

There have been many efforts to determine patient immune endotype using genomic or proteomic biomarkers of immunity (34, 35). While these methods have been helpful in predicting outcomes in sepsis and other disorders (36, 37), in general they have either not been able to provide an accurate assessment of the functional state of host immunity, as it varies over time, or have been used to determine response to therapy. Enzyme-linked immunosorbent spot (ELISpot) is a highly sensitive, functional immunoassay that measures the number of cytokine-secreting cells at the single-cell level in response to ex vivo stimulation (38, 39). A key advantage of ELISpot is that the assay has excellent dynamic range. ELISpot can detect as few as 1 in 100,000 cytokine-secreting cells. Furthermore, ELISpot can test simultaneously the integrity and robustness of the 2 disparate arms of immunity, i.e., innate (blood monocytes and low-density granulocytes) and adaptive cellular immunity (blood lymphocytes) by focusing on the responses of individual cell populations to cell-specific agonists.

The purpose of this study was to determine whether critically ill COVID-19 patients have an exaggerated proinflammatory cytokine storm versus an immunosuppressive immunological endotype, and determine whether there are changes in immune function during disease progression. To provide a comprehensive evaluation, we used conventional flow cytometry to quantitate the effect of COVID-19-mediated depletion of immune effector cells. In addition to quantitating circulating pro- and anti-inflammatory cytokines, we evaluated adaptive and innate immune systems via serial ELISpot assays of T cell IFN- [symbol] and monocyte TNF-α production, respectively.

Results

TABLE 1
Patient demographics.

| | COVID ICU | | Septic | | CINS | | Healthy | |
|---|---|---|---|---|---|---|---|---|
| | patients | | patients | | patients | | volunteers | |
| | (n = 27) | | (n = 51) | | (n = 18) | | (n = 27) | |
| Age, mean (range) | 57 | (25-86) | 56 | (18-89) | 59 | (23-80) | 56 | (25-79) |

Sex

| | | | | | | | | |
|---|---|---|---|---|---|---|---|---|
| Female | 12 | (44%) | 27 | (53%) | 6 | (33%) | 13 | (48%) |
| Male | 15 | (56%) | 24 | (47%) | 12 | (67%) | 14 | (52%) |

Race

| | | | | | | | | |
|---|---|---|---|---|---|---|---|---|
| African American | 19 | (70%) | 11 | (22%) | 2 | (11%) | 7 | (26%) |
| European descent | 8 | (30%) | 40 | (78%) | 16 | (89%) | 20 | (74%) |

Comorbidity

| Hypertension | 17 | (63%) | 9 | (18%) | 10 | (56%) |
|---|---|---|---|---|---|---|
| Diabetes | 11 | (41%) | 19 | (37%) | 0 | |
| Obesity | 8 | (30%) | 4 | (8%) | 2 | (11%) |
| Respiratory disease | 8 | (30%) | 11 | (22%) | 8 | (44%) |
| Cardiovascular disease | 7 | (26%) | 18 | (35%) | 8 | (44%) |
| Neurologic disease | 7 | (26%) | 10 | (20%) | 0 | |
| Hyperlipidemia | 6 | (22%) | 6 | (12%) | 3 | (17%) |
| Thyroid disease | 3 | (11%) | 3 | (6%) | 0 | |
| Cancer | 3 | (11%) | 6 | (12%) | 1 | (6%) |
| Kidney disease | 2 | (7%) | 7 | (14%) | 1 | (6%) |
| Autoimmune disease | 2 | (7%) | 0 | | 0 | |
| Hepatic disease | 1 | (4%) | 6 | (12%) | 0 | |
| Substance abuse | 0 | | 7 | (14%) | 1 | (6%) |
| GI disease | 0 | | 2 | (4%) | 1 | (6%) |
| Days from symptoms to | 6 | (1-14)^{A} | | | | |
| ED, mean (range) | | | | | | |
| Days from ED to | 1 | (0-5)^{B} | | | | |
| intubation, mean (range) | | | | | | |
| Days from ICU to first | 3 | (0-8.5) | | | | |
| blood draw, mean (range) | | | | | | |
| ALC at ICU admission, | 0.9 | (0.4-2.3) | | | | |
| mean (range) | | | | | | |
| APACHE II score, mean | 18 | (6-36)^{C} | 18 | (7-29) | | |
| (range) | | | | | | |
| SOFA score, mean (range) | 7 | (2-14)^{C} | 7 | (0-18) | | |
| Subjects with secondary | 10 | (37%) | 23 | (45%) | | |
| infections | | | | | | |
| 30 day mortality | 10 | (37%) | 11 | (22%) | 2 | (11%) |

^{A}One patient diagnosed with COVID-19 during concurrent admission.
^{B}Four of the 27 subjects did not require intubation during admission.
^{C}Data available for 26 of 27 participants.

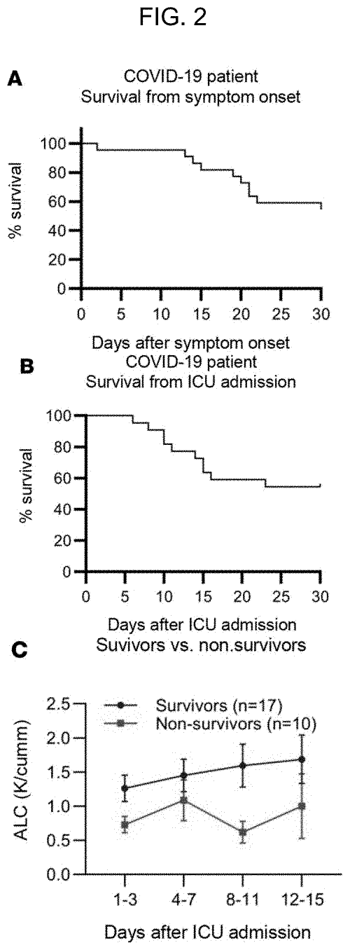

COVID-19 patients were hospitalized in the ICU with a mean of 6 (range 1-14) days after onset of symptoms. Twenty-three of 27 COVID-19 patients were intubated and received invasive mechanical ventilation on average 1 (range 0-5) day from ICU admission. The mean sequential organ failure assessment (SOFA) and APACHE II scores were the equivalent in the COVID-19 and sepsis cohorts (7 and 18, respectively). The 30 day mortality was greater in the COVID-19 group than in patients with sepsis (37% vs. 22%; P=0.14), but the difference did not reach statistical significance. All nonsurviving COVID-19 patients died more than 2 weeks after onset of symptoms and at least 6 days following admission to the ICU (FIG. 2A and FIG. 2B).

The absolute lymphocyte counts (ALC) for COVID-19 patients was 900 cells/mm³, and nonsurvivors had persistent lymphopenia throughout the course of illness compared with COVID-19 survivors (FIG. 2C and TABLE 1). Ten of the 27 COVID-19 patients (37%) had evidence of secondary infections during the first 30 days after enrollment. Thirty percent of patients with secondary infection were nonsurvivors, and 1 patient had coinfection with coronavirus 229E at admission.

Plasma Cytokines.

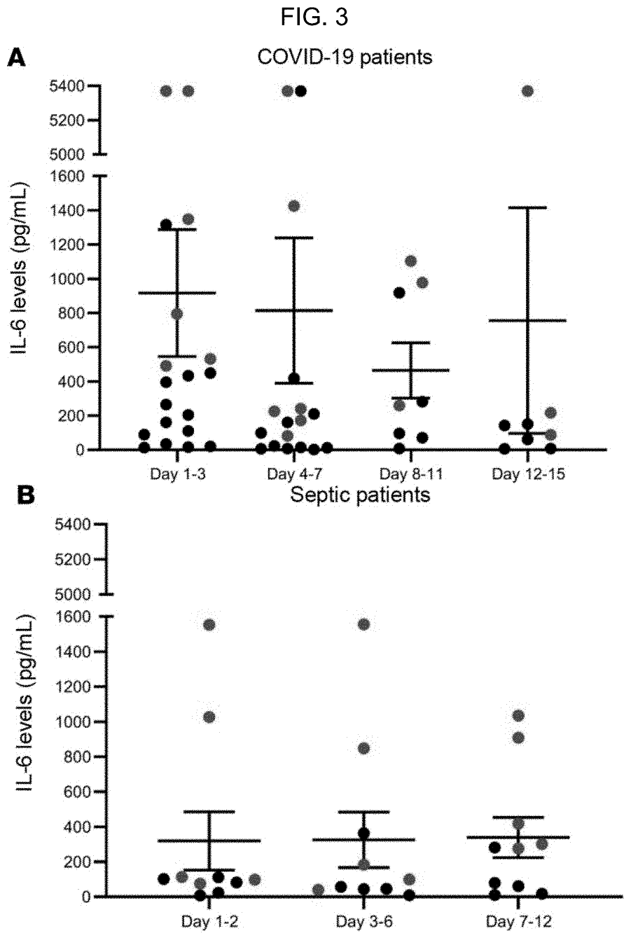

To evaluate the inflammatory response over time, we measured plasma cytokines in COVID-19, septic, and CINS patients and healthy control participants (TABLE 2). Patients with COVID-19 and sepsis patients were followed for up to 4 serial time points after ICU admission. The mean number of sample time points was 2.2 for the COVID-19 patients and 3 for septic patients. A single time point was used for healthy controls and CINS patients. Of note, for COVID-19 patients, the blood sample for cytokine analysis was obtained within the first 24 hours from clinical deterioration (endotracheal intubation) after admission to the ICU in order to try to capture the early hyper-inflammatory phase of infection. Although several key proinflammatory cytokines, including IL-1β, IFN- [symbol] , and TNF-α, were modestly increased in COVID-19 patients compared with healthy control participants, the increases were near the lower limit of detection of the assay (TABLE 2). There was considerable variation in plasma IL-6 levels in COVID-19 patients, with a range from 6 to more than 5000 pg/μL (FIG. 3). IL-6 concentrations were elevated compared with those in healthy controls.

TABLE 2
Plasma cytokines comparing COVID-19 with septic
and CINS patients and healthy volunteers

| | COVID-19 | COVID-19 | COVID-19 | COVID-19 |
|---|---|---|---|---|
| Cytokine | d 1-3 | d 4-7 | d 8-11 | d 12-15 |
| IL-1β Mean | 9.5 (5.4); | 6.0 (4); | 1.3 (0.4); | 3 (1); |
| (SEM); n | 19 | 17 | 8 | 8 |
| IL-6 Mean | 916.4 (381); | 814.1 (437); | 464.4 (172); | 755.6 (705); |
| (SEM); n | 19 | 17 | 8 | 8 |
| IL-7 Mean | 16.5 (6); | 39 (12); | 47.1 (26); | 11.8 (5.5); |
| (SEM); n | 19 | 17 | 8 | 8 |
| IL-8 Mean | 118 (21); | 327.9 (177); | 174.9 (62); | 795.3 (493.5); |
| (SEM); n | 19 | 17 | 8 | 8 |
| IL-10 Mean | 116.6 (65); | 95.4 (59); | 27.2 (12); | 67 (35.2); |
| (SEM); n | 19 | 17 | 8 | 8 |
| IL-12 Mean | 145.2 (48); | 112.1 (21); | 109.6 (43); | 102.9 (37); |
| (SEM); n | 19 | 17 | 8 | 8 |
| MCP-1 Mean | 1394.6 (282); | 1177.3 (257); | 1573.4 (632); | 738.4 (241); |
| (SEM); n | 19 | 17 | 8 | 8 |
| IL-1RA Mean | 333.4 (81); | 497.7 (154); | 323.6 (110); | 646.8 (239); |
| (SEM); n | 19 | 17 | 8 | 8 |
| IFN-γ Mean | 5.9 (2); | 2.1 (1); | 0.5 (0.1); | 0.9 (0.2); |
| (SEM); n | 19 | 17 | 8 | 8 |
| TNF-α Mean | 4.4 (0.5); | 4.3 (1); | 2.6 (1); | 4.6 (3); |
| (SEM); n | 19 | 17 | 8 | 8 |

| | Septic | Septic | Septic | Healthy | |
|---|---|---|---|---|---|
| Cytokine | d 1-2 | d 3-6 | d 7-11 | volunteers | CINS |
| IL-1β Mean | 3.0 (1.2); | 2.3 (0.4); | 3.6 (0.7); | 2 (0.8); | 0.8 (0.3); |
| (SEM); n | 10 | 10 | 10 | 10 | 2 |
| IL-6 Mean | 319.2 (176); | 324.8 (167); | 338.9 (121); | 45.4 (46); | 137.3 (110); |
| (SEM); n | 10 | 10 | 10 | 10 | 4 |
| IL-7 Mean | 62.3 (39); | 65.4 (43); | 65 (34); | 38.7 (8); | 5.5 (0.6); |
| (SEM); n | 10 | 10 | 10 | 10 | 4 |
| IL-8 Mean | 105.9 (55); | 93.7 (33); | 114.8 (47); | 14.5 (1); | 73 (26); |
| (SEM); n | 10 | 10 | 10 | 10 | 4 |
| IL-10 Mean | 881.9 (886); | 677.9 (668); | 685.1 (658); | 226.6 (143); | 25.5 (6); |
| (SEM); n | 10 | 10 | 10 | 10 | 4 |
| IL-12 Mean | 81.1 (33); | 51.4 (11); | 67.5 (15); | 48.4 (6); | 44.4 (15); |
| (SEM); n | 10 | 10 | 10 | 10 | 4 |
| MCP-1 Mean | 507.9 (116); | 491.3 (117); | 686.6 (141); | 512.3 (53); | 414.8 (83); |
| (SEM); n | 10 | 10 | 10 | 10 | 4 |
| IL-1RA Mean | 113.7 (36); | 98.4 (31); | 169.3 (54); | 32.1 (4); | 108.5 (39); |
| (SEM); n | 10 | 10 | 10 | 10 | 4 |
| IFN-γ Mean | 2.4 (0.6); | 2.3 (0.2); | 2.5 (0.3); | 4.4 (0.7); | 9; |
| (SEM); n | 10 | 10 | 10 | 10 | 1 |
| TNF-α Mean | 6.4 (2); | 5.2 (1.6); | 7 (2.5); | 1.4 (0.2); | 6.6 (4); |
| (SEM); n | 10 | 10 | 10 | 10 | 4 |

d 1-3, days 1-3

COVID-19 Induces Profound Suppression of T Cell IFN- [symbol] Production.

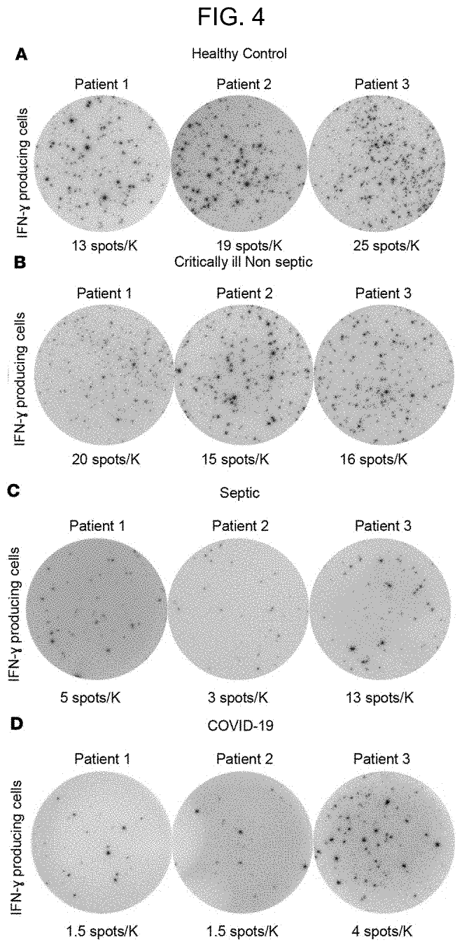

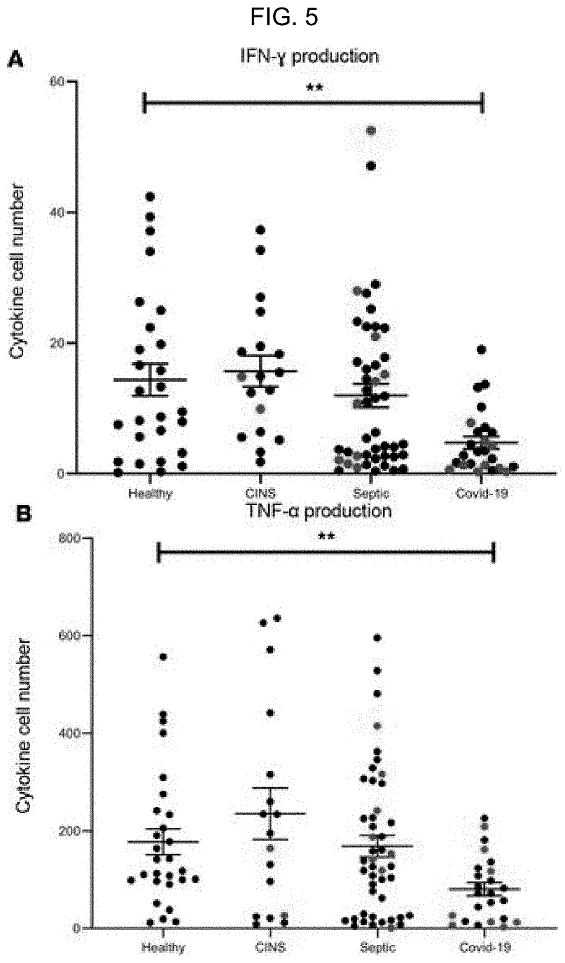

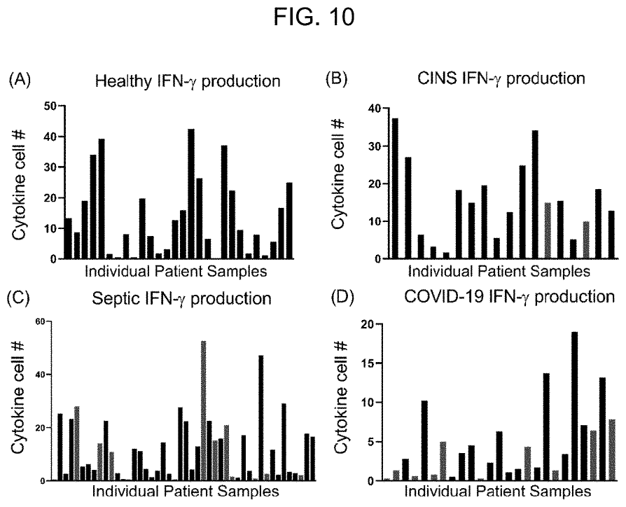

In order to determine the presence and magnitude of functional immunosuppression during COVID-19 infection, we quantitated IFN- [symbol] - and TNF-α-producing cells in overnight cell culture in isolated PBMCs by ELISpot analysis after admission. PBMCs were stimulated and incubated overnight with anti-CD3/anti-CD28 to activate T cells, and IFN- [symbol] -producing cells were quantified. Data are expressed as positive secreting cells per thousand lymphocytes plated. Representative ELISpot figures for IFN- [symbol] -producing cells of representative COVID-19, septic, and CINS patients and healthy volunteers are shown in FIG. 4. Quantitatively, the number of cells producing IFN- [symbol] in patients with COVID-19 infection was significantly reduced compared with those in all the other groups (P=0.004). Stimulated healthy controls had nearly 3-fold more IFN- [symbol] -producing cells than COVID-19 patients (mean 14.4±2.5 vs. 4.8±1). CINS patients had 3-fold-greater levels of stimulated IFN- [symbol] production than COVID-19 patients (mean 15.7±2 vs. 4.8±1). Additionally, the mean number of IFN- [symbol] -producing cells in septic patients was 2-fold greater than in COVID-19 patients (mean 12±2 vs. 4.8±1) (FIG. 5A and FIG. 10).

COVID-19 Induces Profound Suppression of Monocyte TNF-α.

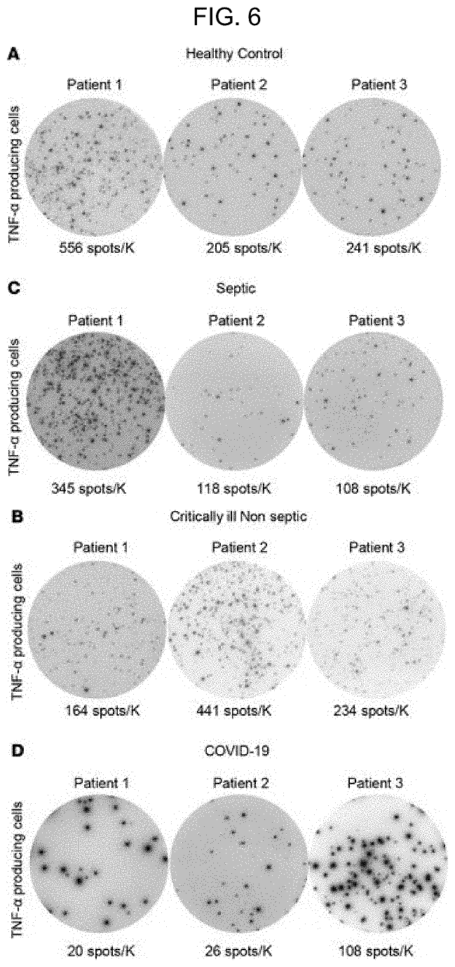

PBMCs were also stimulated overnight with LPS to activate monocytes, and the numbers of TNF-α-producing cells were determined for COVID-19, septic, and CINS patients. Data for TNF-α cytokine-producing cells are expressed as secreting cells per 1000 myeloid cells plated. Representative ELISpot figures for the mean number of TNF-α-producing cells of 3 different COVID-19, septic, and CINS patients and healthy controls are shown in FIG. 6.

Importantly, there was considerable patient heterogeneity in TNF-α production as determined by ELISpot assay. A subset of COVID-19 patients had LPS-stimulated TNF-α production that was comparable to that occurring in other critically ill patients, while a large number of COVID-19 patients had reduced production (FIG. 5B). None of the COVID-19 patients had increased TNF-α production in response to LPS stimulation.

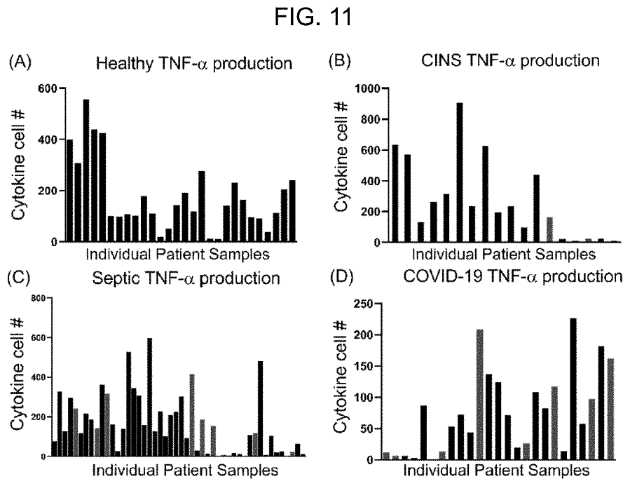

Quantitatively, the number of cells producing TNF-α was reduced 3-fold and 2-fold in with COVID-19 compared with CINS and septic patients, respectively (P=0.009; mean CINS: 272±64; septic: 168±22; COVID-19: 80±14). Compared with healthy volunteers, stimulated PBMCs from COVID-19 patients had half as many TNF-α-producing cells (healthy, 177.5±27) (FIG. 5B and FIG. 11).

Both innate (T cells) and adaptive (monocytes) immune cells from COVID-19 patients who experienced mortality within 30 days of ICU admission were among the most phenotypically suppressed samples. COVID-19 nonsurvivors had quantitatively low ELISpot IFN- [symbol] and TNF-α production, although the difference was not statistically significant (FIG. 5, red dots).

Sustained Immune Suppression Over Time in Patients with COVID-19.

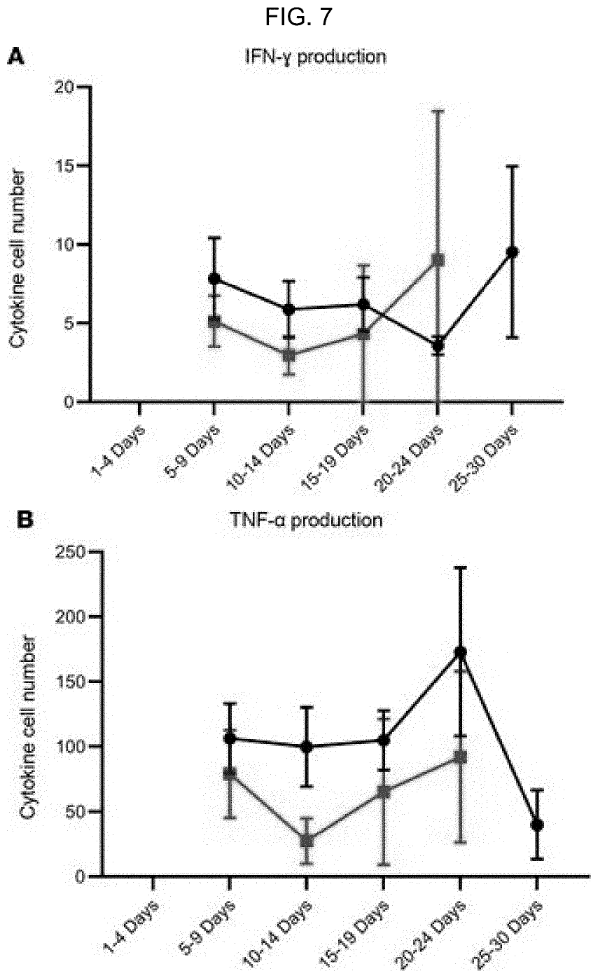

COVID-19 patients were followed over time with serial ELISpot assays, and the mean number of IFN- [symbol] - and TNF-α-producing cells remained suppressed and did not increase over the time course of disease (IFN- [symbol] P=0.54, TNF-α P=0.42) (FIG. 7). Although nonsurvivors maintained lower numbers of IFN- [symbol] - and TNF-α-producing cells than survivors this did not reach statistical significance.

Profound Depletion of CD4⁺ and CD8⁺ T Cells in COVID-19.

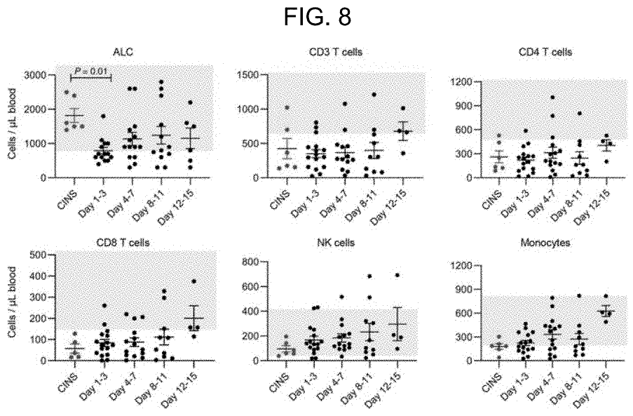

Flow cytometric analysis of samples was performed in all COVID-19 patients (days 1-3, 4-7, 8-11, and 12-15) and in CINS patients (days 1-3) as previously described (33, 34). ALC was profoundly depressed in COVID-19 patients over the entire duration of the study compared with nonseptic patients (first comparison days 1-3; P=0.01) (FIG. 8A). Next, we evaluated absolute CD3⁺, CD4⁺, and CD8⁺ T cell, NK cell, and monocyte numbers (FIG. 8B-FIG. 8F). CD3⁺, CD4⁺, and CD8⁺ T cell numbers were severely depressed compared with those in the normal range (pink shaded area) reported for healthy individuals at the Clinical and Diagnostic CLIA-CLA Laboratories at Barnes-Jewish Hospital (St. Louis, Mo., USA), and remained suppressed for the duration of the study. Although CINS patient samples were not followed sequentially, their initial values were low, similar to the levels found in COVID-19 patients.

IL-7 Increases T Cell IFN- [symbol] Production in COVID-19.

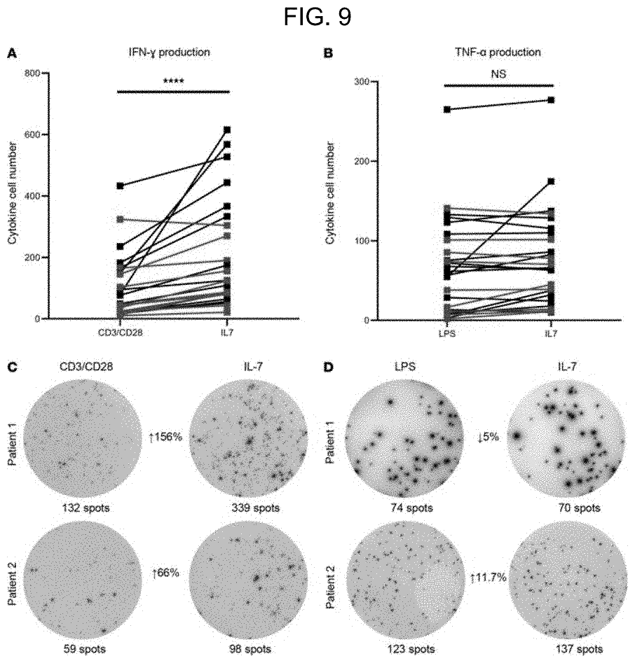

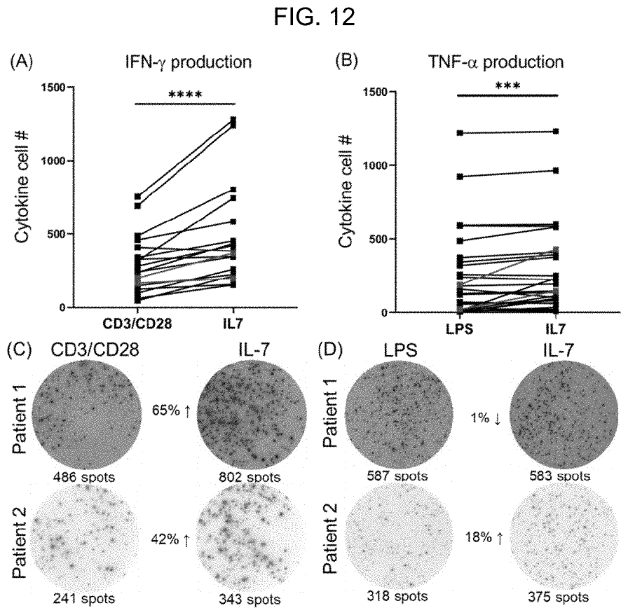

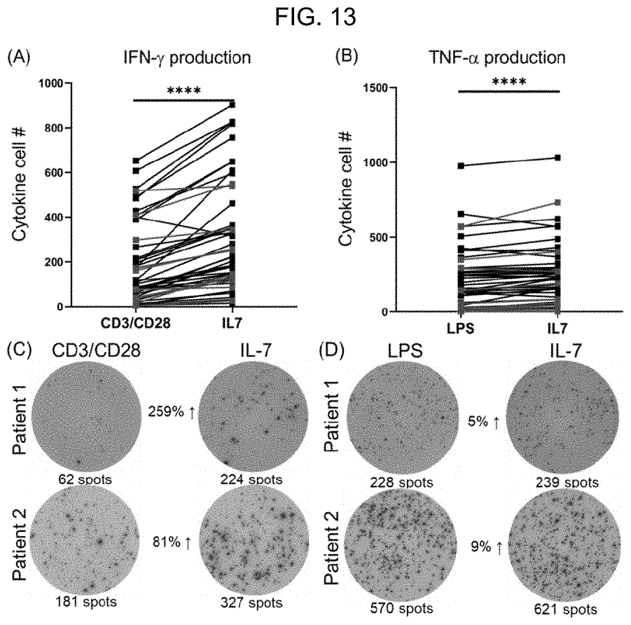

To test the potential efficacy for IL-7 as an immunoadjuvant therapy to restore COVID-19-induced T cell exhaustion, we cocultured patient-derived PBMCs with IL-7 for ELISpot analysis. The mean number of IFN- [symbol] -producing T cells from COVID-19 patients nearly doubled, from 101±21 to 201±36 (P<0.0001), following ex vivo administration of IL-7 (FIG. 9A). Although there was an increase in LPS-induced TNF-α-producing cells in some samples, and a mean increase of 101% overall after IL-7 coincubation, these changes were not statistically significant (FIG. 9B). The effect of IL-7 to increase the number of IFN- [symbol] -producing T cells was also observed in septic and CINS patients (FIG. 12, FIG. 13).

TABLE 3
COVID-19 ICU Patients (n = 27)

| COVID-19 | | | |
|---|---|---|---|
| Patients | Age | Sex | Primary Diagnosis |
| Patient 1 | 57 | M | Acute hypoxemic respiratory failure secondary |
| | | | to adult respiratory distress |
| | | | syndrome due to COVID-19 pneumonia |
| Patient 2 | 57 | F | CAP, viral pneumonia |
| Patient 3 | 40 | F | Acute respiratory distress syndrome (ARDS) |
| | | | due to COVID-19 virus |
| Patient 4 | 67 | M | Acute hypoxemic respiratory failure secondary |
| | | | to ARDS/COVID-19 pneumonia |
| Patient 5 | 47 | M | acute hypoxemic respiratory failure/ARDS secondary |
| | | | to COVID-19 |
| Patient 6 | 52 | F | Pneumonia due to COVID-19 virus |
| Patient 7 | 29 | F | ARDS, COVID19+, transaminitis, metabolic acidosis, |
| | | | lactic acidosis + starvation ketosis, hyponatremia |
| Patient 8 | 49 | M | SIRS, Viral pneumonia vs bacterial, ARF |
| Patient 9 | 84 | M | AHRF, COVID-19, ARDS, |
| Patient 10 | 69 | F | AHRF, COVID19, transaminitis, acute kidney injury |
| Patient 11 | 25 | M | Acute hypoxemic respiratory failure secondary to |
| | | | COVID-19 pneumonia |
| Patient 12 | 33 | F | COVID-19 |
| Patient 13 | 57 | M | COVID-19 virus infection |
| Patient 14 | 61 | M | Pneumonia due to COVID-19 virus |
| Patient 15 | 70 | M | covid-19 viral pneumonia, acute hypoxemic |
| | | | resp failure |
| Patient 16 | 34 | M | Acute hypoxemic respiratory failure due to ARDS, |
| | | | COVID-19 pneumonia |
| Patient 17 | 64 | M | Pneumonia due to COVID-19 virus |
| Patient 18 | 79 | M | Acute hypoxemic respiratory failure: ARDS due to |
| | | | COVID-19 pneumonia |
| Patient 19 | 68 | F | respiratory failure secondary to COVID-19 |
| Patient 20 | 62 | F | Acute hypoxemic respiratory failure, COVID-19, |
| | | | sepsis, acute renal failure |
| Patient 21 | 67 | M | acute hypoxemic respiratory failure/ARDS |
| | | | secondary to COVID-19 |
| Patient 22 | 71 | M | acute hypoxemic respiratory failure/ARDS |
| | | | secondary to COVID-19 |
| Patient 23 | 51 | F | acute hypoxemic respiratory failure/ARDS |
| | | | secondary to COVID-19 |
| Patient 24 | 59 | F | acute hypoxemic respiratory failure/ARDS |
| | | | secondary to COVID-19 |
| Patient 25 | 64 | F | acute hypoxemic respiratory failure/ARDS |
| | | | secondary to COVID-19 |
| Patient 26 | 53 | F | acute hypoxemic respiratory failure/ARDS |
| | | | secondary to COVID-19 |
| Patient 27 | 86 | M | acute hypoxemic respiratory failure/ARDS |
| | | | secondary to COVID-19 |

TABLE 4
Septic Patients (n = 51)

| Septic | | | | | |
|---|---|---|---|---|---|
| Subjects | Age | Sex | Primary Diagnosis | Source of Infection | Organism |
| Patient 1 | 36 | F | Jaundice | PNA | Unknown |
| Patient 2 | 40 | F | Necrotizing Soft Tissue Infection | Necrotizing Fasciitis | Streptococcus agalactiae |
| Patient 3 | 39 | M | Small Bowel Obstruction | Peritonitis | Unknown |
| Patient 4 | 49 | M | Motor Vehicle Collision | Wound Infection | Enterococcus faecium, |
| | | | | | Fusarium, |
| | | | | | Mycobacterium smegmatis, |
| | | | | | Exophiala |
| Patient 5 | 89 | F | Mesenteric Ischemia | Peritonitis | Yeast, Klebsiella pneumoniae, |
| | | | | | Bacteroides fragilis group |
| Patient 6 | 54 | M | Acute Respiratory Failure | PNA | Influenza |
| | | | with Hypoxia | | |
| Patient 7 | 73 | F | Mesenteric Ischemia | Peritonitis | Unknown |
| Patient 8 | 69 | F | Necrotizing Fasciitis | Necrotizing Fasciitis | Escherichia coli, |
| | | | | | Streptococcus anginosus, |
| | | | | | Bacteroides fragilis group |
| Patient 9 | 79 | F | Motor Vehicle Collision | PNA | MRSA |
| Patient 10 | 31 | F | Acute Encephalopathy | PNA | MSSA |
| Patient 11 | 58 | F | Small Bowel Obstruction | Peritonitis | Unknown |
| Patient 12 | 22 | M | Sacral Fracture Closed | UTI | Escherichia coli |
| Patient 13 | 54 | F | Septic Shock | PNA | MRSA |
| Patient 14 | 26 | M | Traumatic Brain Injury | PNA | MSSA |
| Patient 15 | 44 | M | Bowel Perforation | Peritonitis | Escherichia coli, |
| | | | | | Streptococcus constellatus, |
| | | | | | Bacteroides fragilis group |
| Patient 16 | 33 | F | Esophageal Perforation | Mediastinitis | Coagulase negative staphylococcus |
| | | | | | species, Candida albicans |
| Patient 17 | 75 | M | Septic Shock - Urinary Tract | UTI | Proteus mirabilis |
| | | | Infection | | |
| Patient 18 | 54 | M | Spinal Cord Compression | PNA | Yeast |
| Patient 19 | 39 | M | Intraabdominal Fluid Collection | Peritonitis | Coagulase negative |
| | | | | | staphylococcus |
| Patient 20 | 62 | F | Acute Hypoxemic Respiratory Failure | PNA | MSSA |
| Patient 21 | 40 | M | Septic Shock | VAP | MSSA |
| Patient 22 | 33 | M | Pneumoperitoneum | Peritonitis | Coagulase negative |
| | | | | | staphylococcus |
| Patient 23 | 57 | M | Stroke | UTI | Enterobacter cloacae complex |
| Patient 24 | 79 | M | Septic Shock | PNA | Streptococcus pneumoniae |
| Patient 25 | 18 | F | Diffuse Axonal Brain Injury | PNA | MSSA |
| Patient 26 | 82 | F | Acute Hypoxemic Respiratory Failure | PNA | Unknown |
| | | | due to Multifocal Pneumonia | | |
| Patient 27 | 54 | F | Septic Shock | UTI | Pseudomonas Aeruginosa, |
| | | | | | Enterococcus Faecalis |
| Patient 28 | 83 | F | Mixed Shock of Unknown Etiology | Unknown | Unknown |
| Patient 29 | 54 | M | Intraparenchymal Hemorrhage | Peritonitis | Unknown |
| | | M | | | |
| Patient 30 | 63 | | Weakness | Nectrotizing Fasciitis | Enterobacter species |
| Patient 31 | 60 | F | Decompensated Hepatic Cirrhosis | Unknown | Unknown |
| Patient 32 | 59 | M | Acute Respiratory Failure with | PNA | Parainfluenza 3 |
| | | | Hypoxia | | |
| Patient 33 | 72 | M | Spontaneous Bacterial Peritonitis | Peritonitis | Other Staphylococcus species |
| Patient 34 | 59 | M | Hepatic Cirrhosis | PNA | Haemophilus influenza |
| Patient 35 | 68 | F | Respiratory Failure | PNA | Enterobacter species |
| Patient 36 | 46 | M | Acute Liver Failure | Peritonitis | Other Staphylococcus species |
| Patient 37 | 66 | F | GI Bleed | Wound Infection | Acinetobacter, |
| | | | | | Pseudomonas, |
| | | | | | Enterococcus faecium, |
| | | | | | Bacteroides fragilis, |
| | | | | | Stenotrophomonas maltophilia |
| Patient 38 | 57 | M | Respiratory Failure | Peritonitis | Mixed gram positive |
| | | | | | microorganisms |
| Patient 39 | 78 | F | Septic Shock | PNA | Escherichia coli, MRSA |
| Patient 40 | 62 | F | Pneumatosis Coli | Peritonitis | Staphylococcus species |
| Patient 41 | 64 | F | MSSA Bacteremia | Wound Infection | Staphylococcus aureus |
| Patient 42 | 72 | M | Arterial Thromboembolism | Peritonitis | Unknown |
| Patient 43 | 62 | F | Toxic Metabolic Encephalopathy | Peritonitis | Unknown |
| Patient 44 | 59 | F | Acute on Chronic Respiratory | Wound Infection | Staphylococcus epidermidis |
| | | | Failure | | |
| Patient 45 | 28 | F | Diabetic Ketoacidosis | PNA | Unknown |
| Patient 46 | 75 | M | Septic Shock | PNA | Coronavirus HKU1 RNA |
| Patient 47 | 87 | F | Sepsis due to Urinary Tract | UTI | Enterobacter species |
| | | | Infection | | |
| Patient 48 | 66 | F | Perforated Diverticulum | Peritonitis | Unkown |
| Patient 49 | 59 | M | Septic Shock | UTI | Escherichia coli |
| Patient 50 | 59 | F | Septic Shock | PNA | Streptococcus species |
| Patient 51 | 37 | M | Necrotizing Pancreatitis | UTI | Escherichia coli, |
| | | | | | Enterococcus faecalis |

TABLE 5
Critically Ill Non-Septic Patients (n = 18)

| Critically | | | |
|---|---|---|---|
| Ill Patients | Age | Sex | Primary Diagnosis |
| Patient 1 | 57 | M | Polytrauma, Skull Fracture and Subarachnoid |
| | | | Hemorrhage |
| Patient 2 | 36 | M | Surgical Repair for Closed Fracture of |
| | | | Thyroid Cartilage |
| Patient 3 | 56 | M | Pelvic Ring Fracture |
| Patient 4 | 64 | F | Popliteal Arterial Occlusion, left |
| Patient 5 | 56 | M | Motor Vehicle Collision |
| Patient 6 | 23 | F | Desmoid Tumor Resection, paraspinal |
| Patient 7 | 40 | M | Closed Non-displaced Fracture C6 |
| Patient 8 | 61 | M | Retroperitoneal Hematoma, distal aorta |
| Patient 9 | 80 | M | Ruptured Abdominal Aortic Aneurysm |
| Patient 10 | 61 | M | Motor Vehicle Collision |
| Patient 11 | 54 | F | Lumbar Spinal Stenosis with Neurogenic |
| | | | Claudication |
| Patient 12 | 62 | F | Arterial Occlusion |
| Patient 13 | 61 | M | Ruptured Abdominal Aortic Aneurysm |
| Patient 14 | 70 | F | Arterial Occlusion |
| Patient 15 | 62 | M | Popliteal Arterial Occlusion, left |
| Patient 16 | 63 | M | Arterial Occlusion, lower extremity |
| Patient 17 | 75 | M | Central Cord Syndrome |
| Patient 18 | 77 | F | Pulmonary Artery Saddle Embolus |

TABLE 6

| Septic Total 51 | | | Critically III Non-Septic Total 18 | | |
|---|---|---|---|---|---|
| Primary Diagnosis | | | Primary Diagnosis | | |
| Infectious disease | 19 | 37% | Trauma (MVC, Fractures) | 6 | 33% |
| Gastrointestinal disease | 7 | 14% | Peripheral vascular disease | 5 | 28% |
| Respiratory disease | 7 | 14% | Spinal cord injury/disease | 2 | 11% |
| Trauma (MVC, | 5 | 10% | Cardiovascular disease | 2 | 11% |
| Fractures) | | | | | |
| Liver Disease | 4 | 8% | Clotting/Bleeding disorder | 2 | 11% |
| Neurologic disease | 4 | 8% | Neoplasm (nonmalignant) | 1 | 6% |
| Clotting/Bleeding | 2 | 4% | | | |
| disorder | | | | | |
| Metabolic disorder | 1 | 2% | | | |
| Spinal cord | 1 | 2% | | | |
| injury/disease | | | | | |
| Shock | 1 | 2% | | | |
| Source of Infection | | | | | |
| PNA/VAP | 19 | 37% | | | |
| Peritonitis | 15 | 29% | | | |
| UTI | 7 | 14% | | | |
| Wound Infection | 4 | 8% | | | |
| Necrotizing Fasciitis | 3 | 6% | | | |
| Unknown | 2 | 4% | | | |
| Mediastinitis | 1 | 2% | | | |

Discussion

Currently, the prevailing paradigm that guides the therapeutic approach to COVID-19 is that patients are dying from the effects of cytokine storm-mediated inflammation with resultant lung and other organ injury (6, 7, 40-43). Based on this theory of unbridled inflammation, COVID-19 patients are currently being treated with a variety of drugs that block proinflammatory cytokines or inhibit the inflammatory signaling cascade. The results from the present study strongly suggest that the primary endotype of COVID-19 is one of immunosuppression rather than hyper-inflammation. Therefore, the approach of broadly inhibiting the host inflammatory response may be misguided, and may actually worsen clinical trajectories in some COVID-19 patients due to further impairment of an already compromised host protective immune response. Circulating cytokines in COVID-19 patients, at least early in their clinical course, did not show widespread elevation. Most COVID-19 patients had either no elevation or only mild increases in the major proinflammatory cytokines including TNF-α, IL-1α, IL-1β, IFN- [symbol] , etc. (TABLE 2). There were modest elevations in plasma IL-6 in COVID-19 patients, with only 6 patients reaching IL-6 concentrations greater than 1000 pg/μL, as typically seen during overwhelming bacterial sepsis or cytokine release syndrome (44, 45). There were 2 additional COVID-19 patients who had IL-6 levels close to 1000 pg/mL as well as 4 patients whose IL-6 levels were above the level of detection for the assay. Of the aforementioned patients, sustained elevation of IL-6 was detected in some, while others had variable fluctuations in IL-6 levels over time. In addition to macrophages, IL-6 can be made by many different types of cells, including pulmonary epithelial cells, infected with coronaviruses (1, 46). Thus, the increase in IL-6 and IL-8 concentrations that occurs in COVID-19 infection may be a reflection of virus-induced epithelial cell production or cell injury, rather than evidence of a systemic hyper-inflammatory response.

In addition, there was no evidence of exaggerated TNF-α production in response to ex vivo LPS stimulation of PBMCs when compared with septic and CINS patients, nor did the patients have elevated plasma TNF-α levels. Rather, the findings show a predominant endotype of immunosuppression, manifesting as both a profound and sustained loss of CD4⁺ and CD8⁺ T cells, as well as a reduced responsiveness of the remaining lymphocytes to T cell receptor activation. These cells and their responsiveness are essential to containing and eliminating viral pathogens (47). The key finding in the present study is that there is not only a loss in the number of immune cells, but also an accompanying critical defect in the responsiveness of surviving lymphocytes and monocytes.

An aspect of the present study is the use of ELISpot assays performed on freshly obtained blood samples to evaluate individual immune cell responsiveness to agonists. The ELISpot method provides an improved readout of cell function with enhanced sensitivity and increased dynamic range compared with flow cytometric techniques (15, 38). The ELISpot assay showed that when compared with CINS patients, stimulated PBMCs from COVID-19 patients will only activate approximately half the number of IFN- [symbol] -producing lymphocytes (P<0.0001). Similar declines were seen in LPS-stimulated TNF-α production by monocytes from COVID-19 patients. Interestingly, COVID-19 patients who died appeared to have the most profound suppression of TNF-α and IFN- [symbol] production (FIG. 5), and the immune suppression was sustained through at least the first 3 weeks after ICU admission (FIG. 7).

Both clinical and pathological findings suggest that immunosuppression is a critical pathophysiologic phenomenon of COVID-19. Zhou et al. reported that 50% of COVID-19 patients who die develop secondary hospital-acquired infections (48). Autopsy studies of COVID-19 patients demonstrate inclusion bodies, pathologic findings consistent with viral persistence within cells present in lung, kidney, and other organs (23, 49, 50). A recent autopsy investigation of 12 patients who died of COVID-19 showed that 11 of the patients had up to 500,000 viral copies/1×10⁶ RPPH1 copies in lung tissue by SARS-CoV2-specific RT-qPCR (51). Ten of the 12 patients had superimposed bronchopneumonia with both focal and diffuse distribution. Collectively, these studies suggest that there is an inability of the host to mount an adequate immune defense, leading to viral dissemination and organ injury and rendering the patient more susceptible to subsequent hospital-acquired infections.

One important implication of the massive depletion and impaired function of lymphocytes is that immune adjuvants that enhance host immunity should be strongly considered as potential therapeutic interventions in patients with COVID-19. Decades of mechanistic immunologic studies have invariably demonstrated that an intact T cell-mediated adaptive immune response is required for eliminating and suppressing viral infections (52). Support for this potential immune therapeutic approach is provided by studies showing that checkpoint inhibitors and common γ-chain cytokines, which stimulate CD4⁺ and CD8⁺ T cells, have been effective in a number of serious viral infections, including hepatitis C, JC virus-induced progressive multifocal leukoencephalopathy, and HIV (47, 53). Several of these agents (NKG2D-ACE2 CAR-NK cells, anti-PD-1, IL-7) are either in active clinical trials or in the planning stages for COVID-19 (NCT04324996, NCT04356508, NCT04379076, respectively).

Of particular relevance regarding potential immune adjuvant therapy for COVID-19 are the ELISpot results showing that ex vivo IL-7 increased IFN- [symbol] production of stimulated T cells nearly 2-fold (FIG. 9). A clinical trial of IL-7 in patients with sepsis showed that IL-7 was well tolerated, reversed sepsis-induced lymphopenia, and increased CD4⁺ and CD8⁺ T cells by 2- to 3-fold (54).

Another important implication of the present study is that ELISpot may be used to phenotype COVID-19 patients to determine appropriate immunomodulatory drug therapies. Results of the ELISpot analysis showed that some COVID-19 patients displayed ex vivo cytokine production, comparable to results from CINS patients (FIG. 5). Therefore, use of immunostimulant therapies to restore protective immunity in these patients might not be indicated. Conversely, COVID-19 patients with severe reductions in T cell or monocyte cytokine production might benefit from agents to boost their host immunity. We would expect that the ELISpot assay could be used clinically to evaluate the progression of immune dysfunction and to evaluate the effect of different immune therapies to restore innate and adaptive immunity in an immunosuppressed patient.

Most of the COVID-19 patients had symptoms of infection several days prior to hospitalization (FIG. 2). Although an early and excessive hyper-inflammatory phase may have already occurred prior to hospitalization, we deem this unlikely, because significant systemic inflammatory reactions typically induce hypotension and dyspnea that would have led patients to seek immediate care. This study did not exclude a subset of COVID-19 patients who do have cytokine storm-mediated hyper-inflammation with accompanying lung and organ injury. Thus, anti-cytokine therapy or drugs to negatively modulate the inflammatory response may be beneficial in this subset of patients. However, the present results show that a markedly immunosuppressive phenotype predominates in COVID-19 patients. The ongoing clinical trials of anti-cytokine agents and immunosuppressive therapies will likely resolve whether COVID-19 patients actually have damaging hyper-inflammatory responses. Ultimately, in order to eradicate the virus, patients need a competent and active immune system, and research should focus on such therapies to restore this vital function.

Finally, the present results, which are based on blood measurements, do not exclude the possibility that damaging inflammation occurs locally within the lung and other organs that is not detected by levels of circulating cytokines or ELISpot analysis of PBMCs. Direct examination of samples obtained by bronchoalveolar lavage would help address this issue of potential compartmentalized responses to COVID-19 infection.

Conclusions

We conclude that the major immunologic abnormality in COVID-19 is a profound defect in host immunity and not hypercytokinemia-induced organ injury. The defect in host immunity includes both a profound depletion in the number of effector immune cells and severe functional defects in T cell and monocyte function. Based on these findings, immunoadjuvant therapies to enhance host immunity should be considered. Evaluating patient innate and adaptive immunity using functional assays such as ELISpot may be useful in guiding immunomodulatory therapies. IL-7 reverses T cell exhaustion in COVID-19 and should be considered as a potential therapy in this highly lethal disorder.

Methods

Study Design

This was a prospective observational cohort study among patients with COVID-19 in a mixed medical and surgical ICU between March 2020 and May 2020 at Missouri Baptist Medical Center and Barnes-Jewish Hospital. Additionally, samples obtained previously (in 2018-2020) from sepsis or CINS patients were used for comparison.

Patient demographic data, including clinical course, relevant laboratory testing, onset of symptoms prior to admission to the hospital, morbidity, mortality, and medical management data were collected and deidentified. Complete blood counts were recorded at the time closest to blood sampling for immune functional testing. For the COVID-19 patients, the first study blood sample was obtained within the first 24 hours from clinical deterioration (endotracheal intubation) after admission to the ICU in order to try to capture the early hyper-inflammatory phase of infection. COVID-19 patients had 2 blood draws weekly, for a maximum of 4 blood draws, and septic patients had the option for a redraw at 1 week if the patient remained in the ICU.

Inclusion Criteria

We included hospitalized patients, aged 18 years or older, who were COVID-19 positive via either nasopharyngeal- or tracheal aspirate-derived SARS-CoV-2 RNA using an FDA-approved clinical PCR test. COVID-19 testing results were available from 6 to 30 hours after hospital admission. For inclusion in the study, patients with sepsis were defined as previously described (54), including the presence of 2 or more criteria for systemic inflammatory response syndrome (SIRS), 2 or greater point increase in SOFA score, and clinically or microbiologically suspected infection. CINS patients included patients admitted to the medical or surgical ICU following major surgical procedures or major traumatic injury or with noninfectious causes of organ failure, requiring intensive care management and not showing evidence of infection. Healthy control participants had no ongoing infections or autoimmune disease, and no past history of cancer or solid organ transplant.

Exclusion Criteria

No screened patients were excluded from the COVID-19 cohort. For the critically ill groups, to minimize confounding effects of immunosuppressive medications or underlying immunologic disease, patients with the following criteria were excluded: (i) patients with active cancer and/or undergoing chemotherapy or radiation treatment within the past 6 weeks; (ii) HIV; (iii) known history of acute or chronic lymphocytic leukemia; (iv) pregnancy; (v) organ or bone marrow transplantation; (vi) use of current high-dose corticosteroid regimens that were greater than or equivalent to 300 mg/d hydrocortisone or other immunosuppressive medications; (vii) current use of immune-modifying biological agents including inhibitors of TNF-α or other cytokines, viral hepatitis, or systemic autoimmune diseases; and (viii) participation in another interventional trial within the past 4 weeks

Plasma Cytokine Measurements.

Cytokine quantitation was performed on plasma obtained from patients (frozen at −80° C. prior to use), and subsequently analyzed using a human MagPix multiplex cytokine panel (Invitrogen) and on a Luminex FLEXMAP 3D instrument according to the manufacturer's instructions.

ELISpot Quantitation of IFN- [symbol] and TNF-α Production

Quantitation of IFN- [symbol] - and TNF-α-producing cells was performed on isolated PBMCs by ELISpot analysis, as per the manufacturer's instruction (Cellular Technologies Limited [CTL] Immunospot, R&D Systems) and as previously described (38, 39). Patient PBMCs were harvested from whole blood via Ficoll-Paque, counted using the Vi-Cell counter from Beckman Coulter, and incubated overnight plated in 96 well ELISpot culture plates with CLT media or RPMI 1640 media (Sigma-Aldrich) supplemented with human AB serum, nonessential amino acids, penicillin/streptomycin, and I-glutamine. Septic and CINS patient samples were plated in duplicate, and COVID-19 subject samples were plated in triplicate; these results were averaged for each patient sample. ELISpot plates were used for capture of both IFN- [symbol] and TNF-α. For R&D kits, when used, capture antibody was prepared and placed in wells as per the manufacturer's recommendations. CTL kits came with capture antibody precoated. Cells plated in IFN- [symbol] wells were plated at a standardized density of 2.5×10⁴ and 5×10⁴ PBMCs per well and stimulated with anti-CD3 (clone HIT3a, BioLegend) and anti-CD28 (clone CD28.2; BioLegend) antibodies at 1 μg/mL. Cells plated in TNF-α wells were plated at a standardized density of 2.5×10³ and 5×10³ PBMCs per well, and 5×10³ were stimulated with 100 ng/ml LPS (from Salmonella abortus equi S-form, ALX-581-009, Enzo Life Sciences). Anti-CD3 with anti-CD28 or LPS was used as stimulant to evaluate the baseline function of T cells and monocytes, respectively, to assess ability to produce and secrete IFN- [symbol] or TNF-α. ELISpot plates were made by Merck Millipore and obtained through Thermo Fisher Scientific (M8IPS4510). Spots were detected using a colorimetric reagent kit (Strep-AP and BCIP-NBT, R&D Systems, SEL002). Following development, images were captured and analyzed on CTL ImmunoSpot 7.0 plate reader and software.

The immunoadjuvant, IL-7, was obtained from R&D Systems (catalog 207-IL-200). Additional ELISpot wells were prepared as mentioned above with the addition of IL-7 at a final concentration of 50 ng/mL.

Flow Cytometry

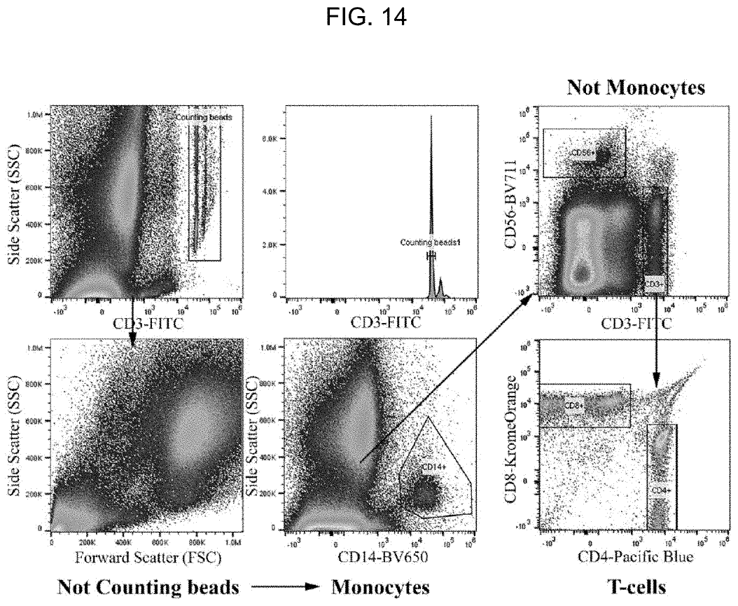

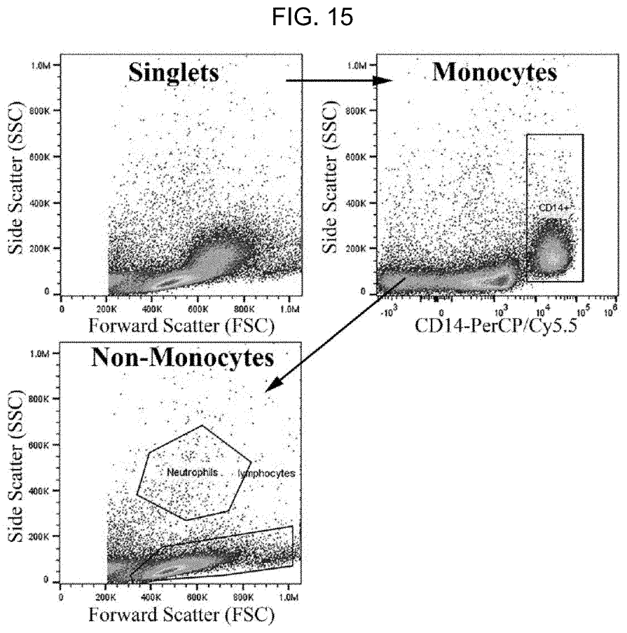

Flow cytometric analysis of samples was performed as previously described (39, 55). Briefly, whole blood or PBMCs were stained for 30 minutes at room temperature, and red blood cells lysed (in the case of whole blood) using Red Blood Cell Lysis Buffer (BioLegend). Samples were acquired on an Attune NxT cytometer (Thermo Fisher Scientific) and data analyzed using FlowJo 10.6.2 (BD Biosciences). Absolute cell counts were ascertained by use of counting beads in LUCID DURAclone staining tubes (Beckman Coulter). The gating strategy used is shown in FIG. 14 and FIG. 15.

The following antibodies (clones) were used in this work: CD3 (HIT3a)-FITC, CD14 (M5E2)-PerCP/Cy5.5, CD4 (RPA-T4)-APC/Cy7, CD8 (SK1)-APC, CD56 (5.1H11)-BV711, CD14 (M5E2)-BV650 (BioLegend), CD3 (UCHT1)-FITC, CD4 (13b8.2)-PacificBlue, and CD8 (B9.11)-KromeOrange (Beckman Coulter).

Statistics

All statistical analyses were performed using GraphPad Prism version 8.4 and SPSS Statistics version 25 (IBM). Mean percentage change in spot number was calculated by dividing the difference between the control and treatment sample by the value of the control. Statistical analysis of ELISpot data comparing unstimulated results with stimulated results was performed using paired analysis with nonparametric Wilcoxon's signed-rank test. In this test, each patient sample is compared with its own unstimulated control, and these changes are compared for the entire group to determine statistical significance. Mann-Whitney U tests were used to compare the mean ELISpot results between different cohorts under similar stimulations. Comparisons of differences in continuous variables within a group (isotype control vs. treatments) were done using paired Student's t tests, 1-way ANOVA, and multivariate analysis. P values less than 0.05 were considered significant.

ELISpot results were corrected for number of cells plated in the following method: The number of spots determined using the CTL ELISpot analyzer represents the number of cells secreting the relevant cytokine. PBMC IFN- [symbol] spots were corrected as the number of spots per lymphocyte percentage in the PBMC fraction based on flow cytometry data. PBMC TNF-α spots were corrected as the number of spots per myeloid cell percentage in the PBMC fraction. For COVID-19 samples, flow cytometry was performed on the PBMC fraction, and neutrophil contamination was included in the correction fraction. Spot number for IFN- [symbol] and TNF-α was reported per thousand cells plated. For samples that did not have flow cytometry data available, complete blood count with differential was used.

### REFERENCES

1. Chen X, et al. Detectable serum SARS-CoV-2 viral load (RNAaemia) is closely correlated with drastically elevated interleukin 6 (IL-6) level in critically ill COVID-19 patients [published online Apr. 17, 2020]. Clin Infect Dis.
2. Guo Y R, et al. The origin, transmission and clinical therapies on coronavirus disease 2019 (COVID-19) outbreak—an update on the status. Mil Med Res. 2020; 7(1):11.
3. McGonagle D, Sharif K, O'Regan A, Bridgewood C. Interleukin-6 use in COVID-19 pneumonia related macrophage activation syndrome. Autoimmun Rev. 2020; 19(6):102537.
4. Qin C, et al. Dysregulation of immune response in patients with coronavirus 2019 (COVID-19) in Wuhan, China. Clin Infect Dis. 2020; 71(15):762-768.
5. Sarzi-Puttini P, et al. COVID-19, cytokines and immunosuppression: what can we learn from severe acute respiratory syndrome? Clin Exp Rheumatol. 2020; 38(2):337-342.
6. Tveito K. Cytokine storms in COVID-19 cases? Tidsskr Nor Laegeforen. 2020; 140
7. Zhang C, Wu Z, Li J W, Zhao H, Wang G Q. Cytokine release syndrome in severe COVID-19: interleukin-6 receptor antagonist tocilizumab may be the key to reduce mortality. Int J Antimicrob Agents. 2020; 55(5):105954.
8. Chatterjee P, et al. The 2019 novel coronavirus disease (COVID-19) pandemic: a review of the current evidence. Indian J Med Res. 2020; 151(2 &3):147-159.
9. Ciotti M, et al. COVID-19 outbreak: an overview. Chemotherapy. 2019; 64(5-6):215-223.
10. Du R H, et al. Hospitalization and critical care of 109 decedents with COVID-19 pneumonia in Wuhan, China. Ann Am Thorac Soc. 2020; 17(7):839-846.
11. Huang C, et al. Clinical features of patients infected with 2019 novel coronavirus in Wuhan, China. Lancet. 2020; 395(10223):497-506.
12. Kang Y J. Mortality rate of infection with COVID-19 in Korea from the perspective of underlying disease [published online Mar. 31, 2020]. Disaster Med Public Health Prep.
13. Wang D, et al. Clinical characteristics of 138 hospitalized patients with 2019 novel coronavirus-infected pneumonia in Wuhan, China. JAMA. 2020; 323(11):1061-1069.
14. Du Y, et al. Clinical features of 85 fatal cases of COVID-19 from Wuhan. A retrospective observational study. Am J Respir Crit Care Med. 2020; 201(11):1372-1379.
15. Remy K E, et al. Immunotherapies for COVID-19: lessons learned from sepsis [published online Apr. 28, 2020]. Lancet Respir Med.
16. Devaux C A, Rolain J M, Colson P, Raoult D. New insights on the antiviral effects of chloroquine against coronavirus: what to expect for COVID-19? Int J Antimicrob Agents. 2020; 55(5):105938.
17. Roback J D, Guamer J. Convalescent plasma to treat COVID-19: possibilities and challenges. JAMA. 2020; 323(16):1561-1562.
18. Shah P, Zampella J G. Use of systemic immunomodulatory therapies during the coronavirus disease 2019 (COVID-19) pandemic. J Am Acad Dermatol. 2020; 82(6):e203-e204.
19. Diao B, et al. Reduction and functional exhaustion of t cells in patients with coronavirus disease 2019 (COVID-19). Front Immunol. 2020; 11:827.
20. Zheng H Y, et al. Elevated exhaustion levels and reduced functional diversity of T cells in peripheral blood may predict severe progression in COVID-19 patients. Cell Mol Immunol. 2020; 17(5):541-543.
21. Zheng M, et al. Functional exhaustion of antiviral lymphocytes in COVID-19 patients. Cell Mol Immunol. 2020; 17(5):533-535.
22. Sereti I, et al. IL-7 administration drives T cell-cycle entry and expansion in HIV-1 infection. Blood. 2009; 113(25):6304-6314.
23. Hanley B, Lucas S B, Youd E, Swift B, Osborn M. Autopsy in suspected COVID-19 cases. J Clin Pathol. 2020; 73(5):239-242.
24. Wang F, et al. Characteristics of peripheral lymphocyte subset alteration in COVID-19 pneumonia. J Infect Dis. 2020; 221(11):1762-1769.
25. Xiong Y, et al. Transcriptomic characteristics of bronchoalveolar lavage fluid and peripheral blood mononuclear cells in COVID-19 patients. Emerg Microbes Infect. 2020; 9(1):761-770.
26. Zheng Y, et al. Study of the lymphocyte change between COVID-19 and non-COVID-19 pneumonia cases suggesting other factors besides uncontrolled inflammation contributed to multi-organ injury. medRxiv. Published Feb. 19, 2020. Accessed Aug. 16, 2020.
27. Peterson D, Damsky W, King B. Reply: Calm before the storm: understanding the role of Janus kinase inhibitors in COVID-19. J Am Acad Dermatol. 2020; 83(1):e67-e68.
28. Russell B, et al. Associations between immune-suppressive and stimulating drugs and novel COVID-19-a systematic review of current evidence. Ecancermedicalscience. 2020; 14:1022.
29. Spinelli F R, Conti F, Gadina M. HiJAKing SARS-CoV-2? The potential role of JAK inhibitors in the management of COVID-19. Sci Immunol. 2020; 5(47):eabc5367.
30. Aouba A, et al. Targeting the inflammatory cascade with anakinra in moderate to severe COVID-19 pneumonia: case series [published online May 6, 2020]. Ann Rheum Dis.
31. Parra-Lopez C A. Phase I/II clinical study of immunotherapy based on adoptive cell transfer as a therapeutic alternative for patients with COVID-19 in Colombia.
ClinicalTrials.gov. https://clinicaltrials.gov/ct2/show/NCT04344548?term=covid+n k+ cells&draw=2&rank=3 Published Apr. 14, 2020. Accessed Aug. 16, 2020.
32. Liu M. A Phase I/II Study of universal off-the-shelf NKG2D-ACE2 CAR-NK cells for therapy of COVID-19.
ClinicalTrials.gov. https://clinicaltrials.gov/ct2/show/NCT04324996?term=covid+n k&draw=2&rank=1 Published Mar. 27, 2020. Accessed Aug. 16, 2020.
33. Ijiri T, Urabe Y, Maeda N, Sasadai J, Suzuki T. Comparative study of the differences in shoulder muscle activation according to arm rotation angle. Hum Mov Sci. 2020; 69:102567.
34. Hayashi N, et al. Multiple biomarkers of sepsis identified by novel time-lapse proteomics of patient serum. PLoS One. 2019; 14(9):e0222403.
35. Sharma N K, Salomao R. Sepsis Through the eyes of proteomics: the progress in the last decade. Shock. 2017; 47(1S suppl 1):17-25.
36. Wong H R, et al. Genomic expression profiling across the pediatric systemic inflammatory response syndrome, sepsis, and septic shock spectrum. Crit Care Med. 2009; 37(5):1558-1566.
37. Oberholzer A, et al. Plasma cytokine measurements augment prognostic scores as indicators of outcome in patients with severe sepsis. Shock. 2005; 23(6):488-493.
38. Mazer M, et al. IL-10 has differential effects on the innate and adaptive immune systems of septic patients. J Immunol. 2019; 203(8):2088-2099.
39. Thampy L K, et al. Restoration of T cell function in multi-drug resistant bacterial sepsis after interleukin-7, anti-PD-L1, and OX-40 administration. PLoS One. 2018; 13(6):e0199497.
40. Jose R J, Manuel A. COVID-19 cytokine storm: the interplay between inflammation and coagulation. Lancet Respir Med. 2020; 8(6):e46-e47.
41. Mehta P, et al. COVID-19: consider cytokine storm syndromes and immunosuppression. Lancet. 2020; 395(10229):1033-1034.
42. Ruscitti P, Berardicurti O, lagnocco A, Giacomelli R. Cytokine storm syndrome in severe COVID-19. Autoimmun Rev. 2020; 19(7):102562.
43. Wright D J M. Prevention of the cytokine storm in COVID-19 [published online May 7, 2020]. Lancet Infect Dis. Waage A, Brandtzaeg P, Halstensen A, Kierulf P, Espevik T. The complex pattern of cytokines in serum from patients with meningococcal septic shock. Association between interleukin 6, interleukin 1, and fatal outcome. J Exp Med. 1989; 169(1):333-338.
45. Tanaka T, Narazaki M, Kishimoto T. Immunotherapeutic implications of IL-6 blockade for cytokine storm. Immunotherapy. 2016; 8(8):959-970.
46. Matsukura S, Kokubu F, Noda H, Tokunaga H, Adachi M. Expression of IL-6, IL-8, and RANTES on human bronchial epithelial cells, NCI-H292, induced by influenza virus A. J Allergy Clin Immunol. 1996; 98(6 pt 1):1080-1087.
47. Swain S L, McKinstry K K, Strutt T M. Expanding roles for CD4+ T cells in immunity to viruses. Nat Rev Immunol. 2012; 12(2):136-148.
48. Zhou F, et al. Clinical course and risk factors for mortality of adult inpatients with COVID-19 in Wuhan, China: a retrospective cohort study. Lancet. 2020; 395(10229):1054-1062.
49. Liu Q, et al. Gross examination report of a COVID-19 death autopsy. Fa Yi Xue Za Zhi. 2020,36(1):21-23.
50. Wichmann D, et al. Autopsy findings and venous thromboembolism in patients With COVID-19 [published online May 6, 2020]. Ann Intern Med.
51. Menter T, et al. Postmortem examination of COVID-19 patients reveals diffuse alveolar damage with severe capillary congestion and variegated findings in lungs and other organs suggesting vascular dysfunction. Histopathology. 2020; 77(2):198-209.
52. Vardhana S A, Wolchok J D. The many faces of the anti-COVID immune response. J Exp Med. 2020; 217(6):e20200678.
53. Hotchkiss R S, Opal S M. Activating immunity to fight a foe—a new path. N Engl J Med. 2020; 382(13):1270-1272.
54. Francois B, et al. Interleukin-7 restores lymphocytes in septic shock: the IRIS-7 randomized clinical trial. JCI Insight. 2018; 3(5):e98960.
55. Drewry A M, et al. Monocyte function and clinical outcomes in febrile and afebrile patients with severe sepsis. Shock. 2018; 50(4):381-387.

### Example 2: A Whole Blood Enzyme-Linked Immunospot Assay for Functional Immune Endotyping of Septic Patients

This example describes that there is significant heterogeneity in the immune response in patients with sepsis. While some septic patients have increased IFN-γ and TNF-α production compared to healthy volunteers, many septic patients have severe suppression of immunity. Septic patients who died had early, severe, and sustained immune suppression as indicated both by a decrease in the number of cytokine producing immune effector cells and a decrease in the amount of cytokine produced on a per cell basis.

Performing the ELISpot assay in patient diluted whole blood is feasible, easier to perform, and more likely to reflect the actual clinical state of the patients' immunity. The ELISpot assay offers a significant advance in the ability to immune phenotype patients with sepsis and to guide therapy of new potential immune adjuvants that are currently being tested in sepsis. The ELISpot assay can have broad clinical applicability in guiding immune therapies in many disorders including patients with autoimmunity, cancer, and patients who have undergone organ transplantation. ELISpot wells coated with various drugs (e.g., tocilizumab, haptoglobin, hemopexin, ox40, IL7, steroids, and many more) have been evaluated using the disclosed platform.

Abstract

Sepsis initiates simultaneous pro- and anti-inflammatory processes, the pattern and intensity of which vary over time. The inability to evaluate the immune status of patients with sepsis in a rapid and quantifiable manner has undoubtedly been a major reason for the failure of many therapeutic trials. Although there has been considerable effort to immunophenotype septic patients, these methods have often not accurately assessed the functional state of host immunity, lack dynamic range, and are more reflective of molecular processes rather than host immunity. In contrast, ELISpot assay measures the number and intensity of cytokine-secreting cells and has excellent dynamic range with rapid turnaround. We investigated the ability of a (to our knowledge) novel whole blood ELISpot assay and compared it with a more traditional ELISpot assay using PBMCs in sepsis. IFN-γ and TNF-α ELISpot assays on whole blood and PBMCs were undertaken in control, critically ill nonseptic, and septic patients. Whole blood ELISpot was easy to perform, and results were generally comparable to PBMC-based ELISpot. However, the whole blood ELISpot assay revealed that nonmonocyte, myeloid populations are a significant source of ex vivo TNF-α production. Septic patients who died had early, profound, and sustained suppression of innate and adaptive immunity. A cohort of septic patients had increased cytokine production compared with controls consistent with either an appropriate or excessive immune response. IL-7 restored ex vivo IFN-γ production in septic patients. The whole blood ELISpot assay offers a significant advance in the ability to immunophenotype patients with sepsis and to guide potential new immunotherapies.

Sepsis is life-threatening organ dysfunction caused by a dysregulated host response to infection (1). Sepsis initiates a complex immunologic response that varies depending upon numerous factors, including patient age, the number and severity of comorbidities, nutritional state, genetics, site of infection, and the particular type of pathogen (2-7). Furthermore, the host immunoinflammatory response will vary in the individual patient over time as the infection persists or resolves. Typically, there is an initial or early proinflammatory phase of sepsis that is accompanied by a more prolonged immunosuppressive phase, often termed immunoparalysis (8-10) or the compensatory anti-inflammatory response syndrome.

The historic large number of unsuccessful clinical trials in sepsis therapeutics have garnered considerable pessimism on the development of potentially new immunomodulatory therapies. Nevertheless, there continue to be several trials underway testing multiple new therapeutics (11-13). Although precision biologic therapy for cancer patients can map each patient's unique tumor mutation profile and therapies for autoimmune diseases can identify and target individual cell type and/or cytokine dysregulation, there remains a void in patient phenotyping for sepsis that would allow for similar application of precise individualized therapies. This void has been compounded by the fact that patients with sepsis often exhibit and transit through several immunological states during the course of their disease, supporting the critical need to functionally endotype individual patients prior to intervention with immunomodulatory drugs. To underscore this need, we have recently seen in the ongoing COVID-19 viral pandemic the failure of several targeted biological response modifiers that further highlights the desperate need for diagnostic tests that can immunophenotype patients (14, 15). Whereas many COVID-19 patients were being treated with drug therapies that block cytokine signaling or suppress immune effector cell function, other COVID-19 patients were being treated with drugs that enhance or restore the immune response. Thus, diametrically opposing therapies were being used in identical COVID-19 cohorts without any approach that could reveal their immunologic phenotype. For the application of new immunomodulatory therapies in sepsis to succeed, there is a critical need for a diagnostic modality that can both determine the functional state of the patient's immune system in a quantifiable manner as well as evaluate the effectiveness of potential immune restorative therapies.

There have been many efforts to develop predictive indices and to identify specific immune phenotypes for patients with sepsis using genomic or proteomic biomarkers of immunity. Although these methods have been helpful in predicting outcomes in sepsis, in general, they have not been able to provide an accurate assessment of the functional state of host immunity and are generally more reflective of past cellular or molecular responses rather than the present state of the subject's immune response (16). The ELISpot is a highly sensitive immunoassay that measures the ex vivo frequency of cytokine-secreting cells at the single cell level (17-20). A key advantage of ELISpot is that the assay has an excellent dynamic five-log range, enabling it to accurately define the immune dysfunction. In addition to detecting the number of cytokine-secreting cells, the relative amount of cytokine that is produced by each cell can be determined by measuring the total well intensity (TWI) as a function of the total area of counted spots and the pixel density of each spot.

An additional advantage of the ELISpot assay is its ability to independently assess the function of the two major arms of the immune system, namely the innate and adaptive response (21-24). This ability to selectively assess the function of both innate and adaptive immunity is particularly important because sepsis is widely considered to cause an initial potent activation of innate immunity and an early suppression of adaptive immunity. Precise knowledge of the functional state of innate and adaptive immunity will permit the identification of individual sepsis patients who may benefit from new immunomodulatory drug therapies that selectively target key innate and adaptive immune effector cells (25).

Current ELISpot protocols require the isolation of PBMCs prior to ex vivo stimulation. The purpose of this investigation was to establish a novel whole blood ELISpot method to determine the functional immune status (i.e., proinflammatory versus immunosuppressive) in critically ill patients with sepsis. Successful development of an ELISpot assay using patient whole blood can greatly simplify assay performance and would generate findings that are much more likely to reflect the actual immunologic state of the patient's immune response because the assay is performed in the presence of the patient's own plasma and includes all leukocyte populations. IFN-γ production was used to assess adaptive immune function and TNF-α as an indicator of the innate response. IFN-γ was selected as the T cell cytokine of interest because of its central role in host defense, and loss of T cell IFN-γ production is a hallmark of “exhausted” T cells in patients with sepsis (26). TNF-α was selected as an indicator of the state of TLR4-mediated innate immune function because TNF-α is one of the major cytokines produced by activated myeloid cells (27). The results of the ELISpot assays for IFN-γ and TNF-α were obtained serially throughout the hospital course in septic patients to determine the differential effects on innate and adaptive immune function over time and to relate changes with clinical metrics. Finally, we tested the ability of potential immune therapies ex vivo to restore the immune effector cell function using the whole blood ELISpot assay. It was therefore hypothesized that use of the whole blood ELISpot assay could both uncover key functional immune endotypes of patients with sepsis and serve as a viable platform for evaluating the efficacy of different immunotherapies.

Materials and Methods

Study Design

This prospective, observational, ex vivo study was performed on adult patients with sepsis, adult patients with critical illness without sepsis, and healthy volunteers acquired at Barnes Jewish Hospital (Washington University School of Medicine, St. Louis, Mo.). The study was approved by the Human Research Protection Office (Institutional Review Board approval no.: 201603006 and 201808049). Informed consent for participation was provided by all patients or their legally authorized representatives.

Inclusion Criteria

Patients hospitalized in the intensive care unit who were 18 y of age or greater were eligible for enrollment. Sepsis was defined based on the 2016 Third International Consensus Conference definition for sepsis and septic shock (Sepsis-3) (1). Patients with a change of two points or greater using the sequential organ failure assessment (SOFA) scoring system were included. In addition, enrolled patients had a clinically suspected or microbiologically proven infection. Control subjects consisted of 1) a cohort of critically ill nonseptic (CINS) patients who were admitted to the intensive care unit for noninfectious causes and 2) a cohort of healthy nonhospitalized subjects.

Exclusion Criteria

To minimize the potential confounding effects by immune altering conditions, subjects having any one of the following criteria were excluded: 1) immune-altering chronic infectious diseases such as HIV or chronic hepatitis, 2) immunosuppressive medications including chemotherapy or radiation treatment within the previous 6 wk, 3) current use of high-dose corticosteroid regimens defined as exceeding greater than a dose of 300 mg of hydrocortisone or its equivalent, 4) immune-modifying biological agents or other immunosuppression transplant-associated medications, and 5) patients with systemic autoimmune diseases.

Blood Sampling and Processing

Patients consented for up to three blood samples obtained serially in sodium heparin tubes. The initial blood sample from septic patients was drawn within the first 24-48 h of sepsis diagnosis. Subsequent blood draws occurred on days 3-5 and 6-10 for up to three samples if the patient remained in the hospital.

Fractionation of PBMCs

Fresh whole blood samples were processed within 90 min of collection as previously described (28). Briefly, blood was diluted in an equal volume of PBS and layered carefully on Ficoll Paque PLUS (GE Healthcare). The PBMC fraction was isolated following centrifugation at 500×g for 30 min at room temperature. The number of total PBMCs was determined with a Vi-CELL Viability Analyzer (Beckman Coulter, Brea, Calif.). Flow cytometry was performed on PBMC fraction for cell typing.

Preparation of ELISpot Assay for Assessment of Adaptive and Innate Immune Function

Innate and adaptive immune function was assessed using ELISpot analysis by measurement of the production of IFN-γ and TNF-α in ex vivo-stimulated cells following overnight culture. Capture Ab precoated 96-well polyvinylidene difluoride-backed strip plates were used for single color enzymatic assays (ImmunoSpot; Cellular Technology [CTL], Cleveland, Ohio) for detection of human IFN-γ and TNF-α. ELISpot culture procedure was followed as directed using instructions from the ELISpot kit. Samples were run in duplicate for each test condition. Plates were prepared with stimulant and were incubated at 37° C. and 5% CO₂ for 30 min prior to plating cells. Identical conditions were prepared for comparison of whole blood assay to PBMC assay, and culture media alone was used as a negative control. Combination of 500 ng/ml of anti-CD3 (clone HIT3a; BioLegend) with 2.5 μg/ml of anti-CD28 (clone CD28.2; BioLegend) Abs were used to induce IFN-γ, and 2.5 ng/ml LPS (from Salmonella abortus equi S-form, ALX-581-009; Enzo Life Sciences, Farmingdale, N.Y.) was used to induce TNF-α wells. Total well volume for all samples was 200 μl. PBMCs were plated into wells in quantities of 2.5×10⁴ cells per well for IFN-γ and 2.5×10³ cells per well for TNF-α. The relevant volume for 5×10⁴ leukocytes of diluted whole blood (in culture media) was plated in each well based on complete blood counts performed in the clinical research core laboratory at Washington University. PBMCs and diluted whole blood were costimulated with and without recombinant human IL-7 (Escherichia coli-derived protein, product no. 207-IL; R&D Systems, Minneapolis, Minn.). ELISpot assays were incubated overnight for 18-22 h at 37° C. and 5% CO₂ as previously described (29). Following overnight incubation, a biotinylated secondary detection Ab, streptavidin-bound alkaline phosphatase, and developer solution were applied to samples as per manufacturer instructions prior to image capture and analysis.

ELISpot Analysis

Samples were scanned, analyzed, and quality controlled for spot count, spot area, and TWI using a Cellular Technology series 6 ImmunoSpot Universal Analyzer with ImmunoSpot 7.0 professional software (Cellular Technology Analyzers, Shaker Heights, Ohio). ELISpot analysis parameters were optimized to obtain appropriate spot numbers (cytokine-secreting cells) and were maintained constant throughout each sample.

Evaluation of Cytokine Production Based Upon Number of Spot-Forming Units

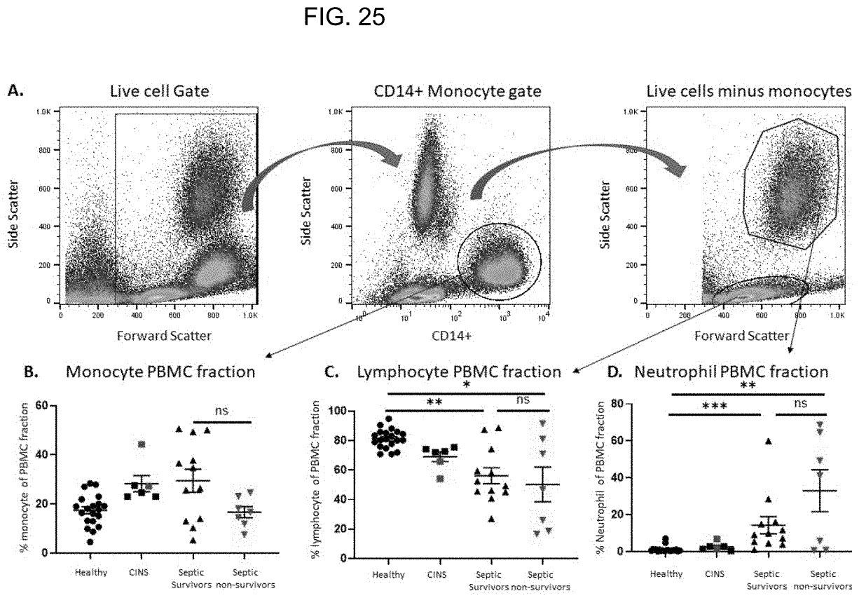

The number of cytokine-secreting cells present in each ELISpot well is referred to as spot-forming units (SFU) and is reported in two distinct ways. SFUs are reported as spots per microliter of diluted whole blood and as spots per 1000 lymphocytes (for IFN-γ ELISpot) or as spots per 1000 myeloid (monocytes and neutrophils) cells (for TNF-α ELISpot). The number of lymphocytes in each well was determined based upon the absolute lymphocyte count as measured by the patients' complete blood count. For ELISpot studies involving PBMCs, flow cytometry was performed on the PBMC fraction, and the number of lymphocytes, monocytes, and residual neutrophils were determined. Cells were stained for CD14 (clone M5E2; BioLegend, San Diego, Calif.). Samples were acquired on FACScan (Becton Dickenson, Franklin Lakes, N.J.) with a five-color modification (CyTek Biosciences, Fremont, Calif.) and analyzed using FlowJo 10.2 (FlowJo, Ashland, Oreg.) as previously described (28). Gating strategy (shown in FIG. 25) consisted of drawing a preliminary size gate on a forward×side scatter plot. This gate was interrogated for CD14 positivity (monocytes). A subsequent gate that excluded the monocytes (Boolean “not” gate) was used to determine lymphocyte and granulocyte population percentages on a forward×side scatter plot.

Evaluation of Cytokine Production Based Upon Spot Intensity

In addition to the number of cytokine-producing cells, data are also reported using an automated analytical method (Cellular Technology ImmunoSpot 7.0 software) based upon the pixel density/intensity of each ELISpot well with adjustment for background well intensity (23, 30). The intensity of each well was calculated based upon the total area of the well encompassed by spots with a correction for the background intensity of each well. This analytical method allows easy interassay variability of wells from different experimental settings to be accurately assessed. The mean intensity is then multiplied by the proportion of the well that is covered in spots (total foreground area [×10³ mm²/total well area]) to establish the TWI. This metric, presented as a percentage of the maximum intensity, is a comparable measurement to the results obtained by ELISA for ex vivo-stimulated cytokine production. The total intensity is reported in this article multiplied by 10² for ease of expression. TWI is normalized and reported as TWI per microliter of whole blood as well as per 1000 lymphocytes (IFN-γ) or 1000 myeloid cells (TNF-α).

Determining Contribution of Monocytes Versus Neutrophils to ELISpot TNF-α Via Cell Depletion

In additional whole blood measurements, RBCs were eliminated from the blood sample using EasySep RBC Depletion Reagent (STEMCELL Technologies, Vancouver, BC, Canada), according to the manufacturer's directions. After completing RBC depletion, monocytes were selectively removed using the Human Monocyte Isolation Kit (STEMCELL Technologies). The final product was a whole blood solution without RBCs or monocytes. Samples were washed and reconstituted in culture media with native patient plasma. Purity of the sample was confirmed using flow cytometry.

Assay of Cytokines and Chemokines

Cytokine quantitation was performed on previously frozen plasma using a human MagPix multiplex cytokine panel (Invitrogen) and analyzed on a Luminex FLEXMAP 3D instrument, according to the manufacturer's instructions. Cytokines in the 35-plex panel included EGF, Eotaxin, FGF-basic, G-CSF, GM-CSF, HGF, IFN-α, IFN-γ, IL-1β, IL-1α, IL-1RA, IL-2, IL-2R, IL-3, IL-4, IL-5, IL-6, IL-7, IL-8, IL-9, IL-10, IL-12 (p40/p70) IL-13, IL-15, IL-17A, IL-17F, IL-22, IP-10, MCP-1, MIG, MIP-1α, MIP-1β, RANTES, TNF-α, and VEGF.

Statistical Analysis

ELISpot samples were performed in duplicate, and results from the two wells were averaged. ELISpot data were analyzed using GraphPad Prism version 8.4 (GraphPad, San Diego, Calif.). Analysis of differences between groups was performed using a nonparametric Kruskal-Wallis test with multiple comparisons corrected by controlling for the desired false discovery rate (FDR) of 5% using the Benjamini, Krieger, and Yekutieli method (31). The FDR corrected p values are reported with p=0.05 indicating statistical significance. Statistical analysis of the change in cytokine production with and without ex vivo IL-7 was analyzed using the Wilcoxon signed-rank test.

Results

Demographic Characteristics/Clinical Parameters

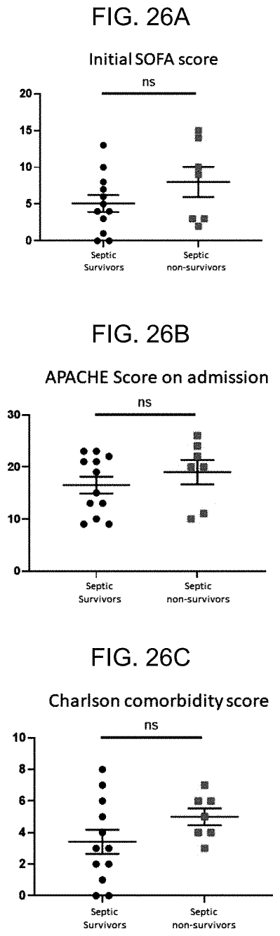

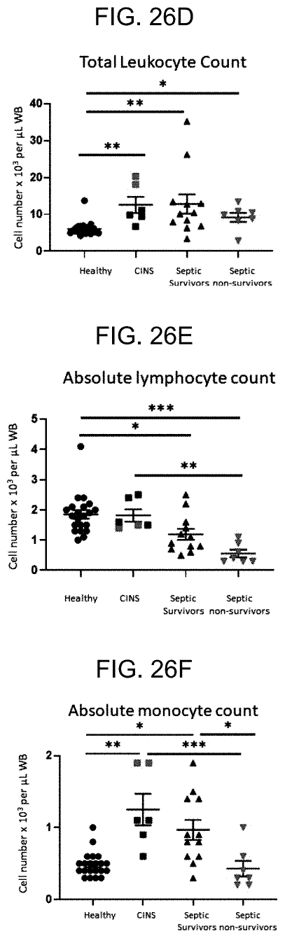

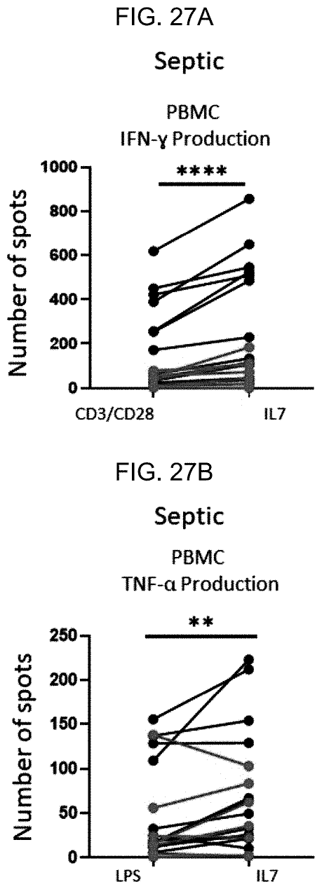

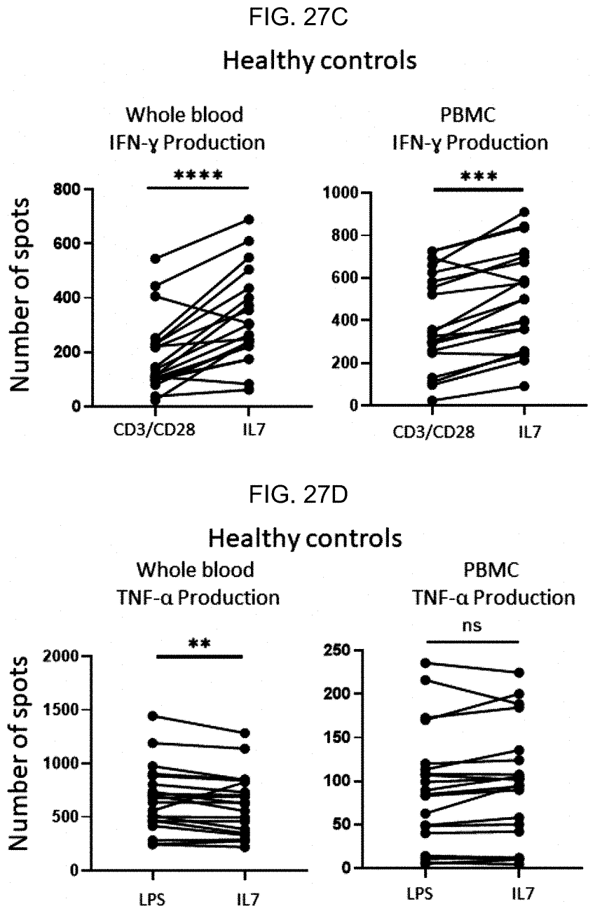

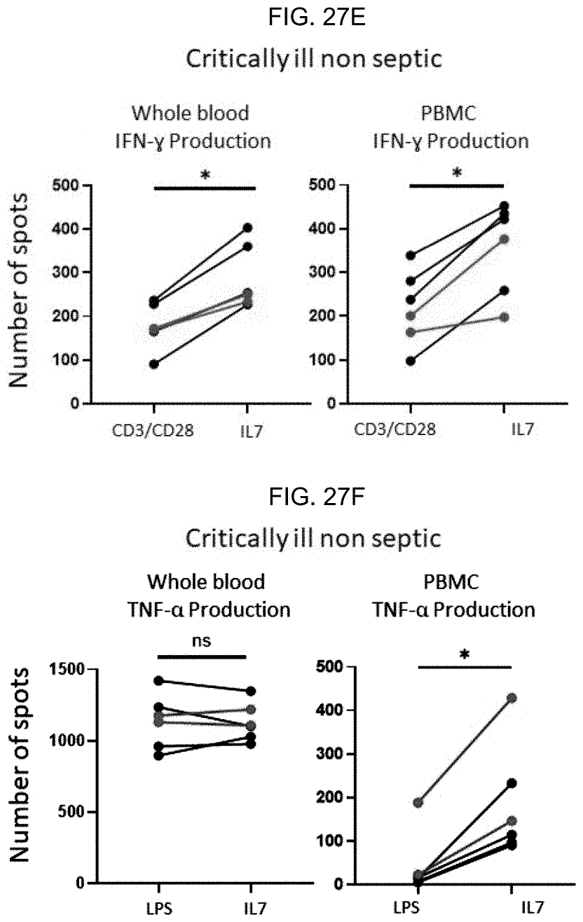

The relevant clinical and laboratory data for the 19 septic, six CINS, and 20 healthy control subjects are presented in TABLE 7. The average Acute Physiology and Chronic Health Evaluation-II and SOFA scores for the septic patients were 17±1 and 6±1, respectively. Of note, there was no significant difference between sepsis survivors and nonsurvivors in terms of severity of illness or comorbidity scores (FIG. 26). The in-hospital mortality was 37% (7/19) and 33% (2/6) for septic and CINS patients, respectively. A list of primary diagnoses for individual patients with sepsis and for CINS patients can be found in TABLE 8.

TABLE 7
Patient demographics

| | Septic | | CINS | | Healthy | | |
|---|---|---|---|---|---|---|---|
| | Patients | | Patients | | Controls | | |
| Demographics | (n = 19) | | (n = 6) | | (n = 20) | | p Value |
| Age, mean (range) | 59 | (28-87) | 48 | (23-64) | 51 | (25-69) | 0.06 |
| Sex | | | | | | | |
| Female | 12 | (63%) | 2 | (33%) | 13 | (65%) | 0.36 |
| Male | 7 | (37%) | 4 | (67%) | 7 | (35%) | |
| Race | | | | | | | |
| African American | 8 | (42%) | 2 | (33%) | 3 | (15%) | 0.49 |
| White | 11 | (58%) | 4 | (67%) | 15 | (75%) | |
| Asian | 0 | | 0 | | 1 | (5%) | |
| Hispanic | 0 | | 0 | | 1 | (5%) | |
| Comorbidities | | | | | | | |
| Cancer | 2 | (11%) | 1 | (17%) | | | |
| Cardiovascular disease | 9 | (47%) | 1 | (17%) | | | |
| Diabetes | 8 | (42%) | 0 | | | | |
| Gastrointestinal disease | 1 | (5%) | 0 | | | | |
| Hepatic disease | 1 | (5%) | 0 | | | | |
| Hyperlipidemia | 2 | (11%) | 1 | (17%) | | | |
| Hypertension | 2 | (11%) | 1 | (17%) | | | |
| Kidney disease | 5 | (26%) | 1 | (17%) | | | |
| Neurologic disease | 2 | (11%) | 0 | | | | |
| Obesity | 4 | (21%) | 0 | | | | |
| Respiratory disease | 6 | (32%) | 2 | (33%) | | | |
| Substance abuse | 1 | (5%) | 1 | (17%) | | | |
| Thyroid disease | 1 | (5%) | 0 | | | | |
| APACHE II score, mean (range) | 17 | (9-26) | 11 | (3-21) | | | 0.04 |
| SOFA score, mean (range) | 6 | (0-15) | 4.3 | (1-9) | | | 0.46 |
| Charlson comorbidity score | 4 | (0-8) | 2 | (0-7) | | | 0.1 |
| Subjects with secondary | 4 | (11%) | N/A | | | | |
| infections | | | | | | | |
| In-hospital mortality | 7 | (37%) | 2 | (33%) | | | |
| WBC count × 10³ per μl (initial | 11.5 | (3.4-35.2) | 12.6 | (6.7-20.3) | 6 | (4.2-13.7) | 0.001 |
| blood draw) | | | | | | | |
| Absolute lymphocyte count × | 1.04 | (0.3-2.5) | 1.82 | (1.4-2.5) | 1.89 | (1.1-4.1) | 0.0008 |
| 10³ per μl (initial blood draw) | | | | | | | |
| Absolute monocyte count × | 0.77 | (0.2-1.9) | 1.25 | (0.6-1.9) | 0.49 | (0.3-1) | 0.003 |
| 10³ per μl (initial blood draw) | | | | | | | |

TABLE 8
Septic Patient Etiologies

| | | Primary | Source of | |
|---|---|---|---|---|
| Cohort | Subjects | Diagnosis | Infection | Organism |
| Sepsis | Patient 1 | GI Bleed | Wound Infection | Acinetobacter, Pseudomonas, |
| | | | | Enterococcus faecium, |
| | | | | B. Fragilis, Stenotrophomonas |
| | | | | maltophilia |
| Sepsis | Patient 2 | Respiratory Failure | Peritonitis | Mixed gram positive microorganisms |
| Sepsis | Patient 3 | Septic Shock | PNA | Escherichia coli, MRSA |
| Sepsis | Patient 4 | Pneumatosis Coli | Peritonitis | Staphylococcus species |
| Sepsis | Patient 5 | MSSA Bacteremia | Wound Infection | Staphylococcus aureus |
| Sepsis | Patient 6 | Arterial Thromboembolus | Peritonitis | Unknown |
| Sepsis | Patient 7 | Toxic Metabolic Encephalopathy | Peritonitis | Unknown |
| Sepsis | Patient 8 | Acute on Chronic Respiratory Failure | Wound Infection | Staphylococcus epidermidis |
| Sepsis | Patient 9 | Diabetic Ketoacidosis | PNA | Unknown |
| Sepsis | Patient 10 | Septic Shock | PNA | Coronavirus HKU1 RNA |
| Sepsis | Patient 11 | Sepsis due to Urinary Tract Infection | UTI | Enterobacter species |
| Sepsis | Patient 12 | Perforated Diverticulum | Peritonitis | Unknown |
| Sepsis | Patient 13 | Septic Shock | UTI | Escherichia coli |
| Sepsis | Patient 14 | Septic Shock | PNA | Streptococcus species |
| Sepsis | Patient 15 | Necrotizing Pancreatitis | UTI | Escherichia coli, Enterococcus faecalis |
| Sepsis | Patient 16 | Leg Ulceration | Peritonitis | Escherichia coli |
| Sepsis | Patient 17 | Syncope | PNA | Unknown |
| Sepsis | Patient 18 | Altered mental status | Central Nervous | Unknown |
| Sepsis | Patient 19 | Gunshot Wound | PNA | Unknown |

| | | |
|---|---|---|
| CINS | Patient 1 | Polytrauma, Skull Fracture and Subarachnoid |
| | | Hemorrhage |
| CINS | Patient 2 | Surgical Repair for Closed Fracture of |
| | | Thyroid Cartilage |
| CINS | Patient 3 | Pelvic Ring Feature |
| CINS | Patient 4 | Popliteal Arterial Occlusion, left |
| CINS | Patient 5 | Motor Vehicle Collision |
| CINS | Patient 6 | Desmoid Tumor Resection, |
| | | paraspinal |

The WBC counts (cells×1000/μl) were higher in both CINS (12.6±2.4; p<0.005), septic survivors (12.8±2.7; p<0.005), and septic nonsurvivors (9.2±1.3; p<0.05) compared with healthy control subjects (6.1±0.5). Conversely, the absolute lymphocyte count (cells×1000/μl) in septic patients who died (0.6±0.1) was significantly decreased compared with healthy controls (1.9±0.2; p<0.0005) and CINS (1.8±0.2; p<0.01) but not septic survivors (1.3±0.2). The number of monocytes (cells×1000/μl) was increased in septic survivors (1.0±0.1; p<0.05) and CINS (1.3±0.2; p<0.01) compared with both sepsis nonsurvivors (0.4±0.1) and healthy control (0.5±0.04) subjects. Sepsis nonsurvivors had a similar monocyte count to healthy controls (FIG. 26).

Unstimulated Whole Blood Production of IFN-γ and TNF-α in Patients with Sepsis

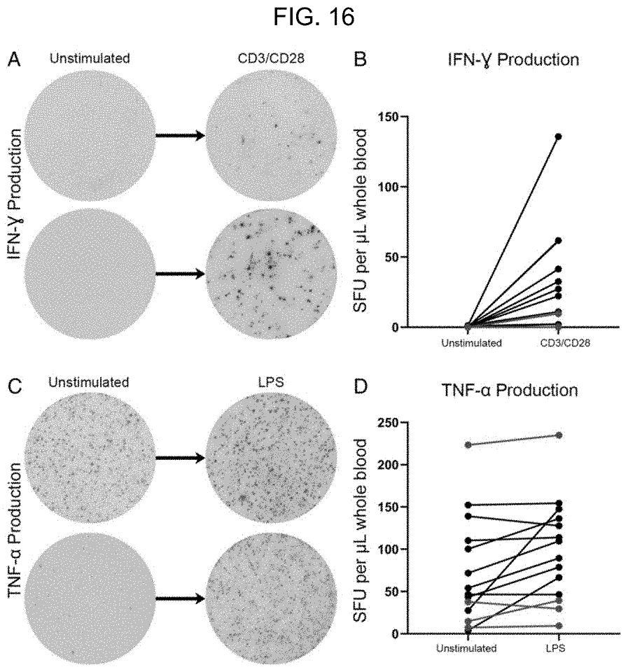

Data from ex vivo production of IFN-γ and TNF-α in whole blood unstimulated with either anti-CD3/CD28 or LPS are shown in FIG. 16 for the first 15 of the 19 septic patients in the cohort. There was essentially no production of IFN-γ without stimulation by CD3/CD28-activating Abs. This lack of cytokine production was true not only for whole blood ELISpot but also for ex vivo IFN-γ production in PBMCs (data not shown). In contrast to IFN-γ, septic patient samples produced spontaneous, unstimulated TNF-α (i.e., without the addition of LPS). Among septic patients, two distinct groups can be characterized based on low (<100 SFU/μl) or high (>100 SFU/μl) spontaneous TNF-α production (nine versus five patients, respectively). In both groups, there was also a subset of patients who had at least a 20% increase in the number of TNF-α-producing cells following LPS stimulation compared with spontaneous production, as well as a subset of patients who did not respond to LPS stimulation above their baseline. Intriguingly, three of the four septic patients who failed to demonstrate an increase in TNF-α production with LPS stimulation died versus only one of nine septic patients who did have a response to LPS.

Suppressed ELISpot IFN-γ Production is Associated with Sepsis Mortality

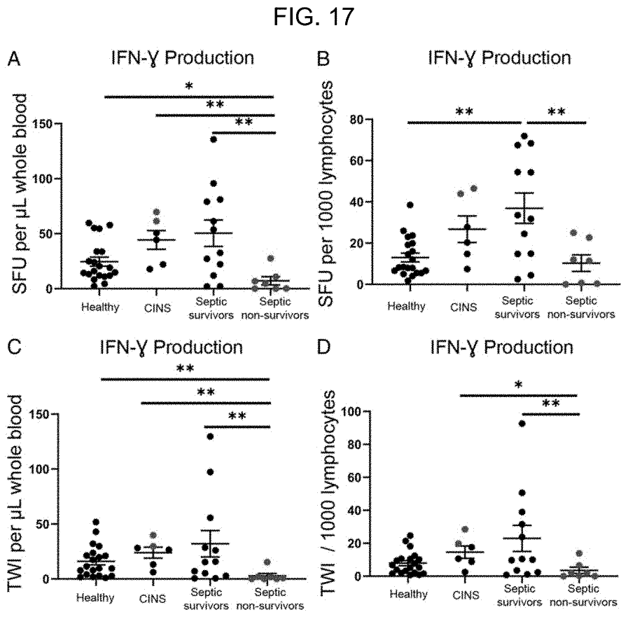

Whole blood IFN-γ production after CD3/CD28 stimulation is shown in FIG. 17. Healthy volunteers and CINS patient responses are compared with the immune function of sepsis within 48 h after sepsis diagnosis. Number of cytokine-producing cells (SFU) and overall cytokine production as TWI was measured. Mean number of IFN-γ-producing cells per microliter of blood was 25±4 for healthy volunteers and 44±9 for CINS versus 50±12 and 7±4 for septic survivors and septic nonsurvivors, respectively (FIG. 17A). Per microliter of blood, sepsis nonsurvivors had significantly lower IFN-γ-producing cells and TWI compared with healthy controls (p<0.05), CINS (p<0.01), and sepsis survivors (p<0.01) (FIG. 17A, FIG. 17C). Patients who died of sepsis had 3-fold lower numbers of activated T cells per lymphocyte compared with patients who survived sepsis (10±4 for nonsurvivors, 37±8 for survivors; p<0.01) (FIG. 17B). Intensity per 1000 lymphocytes was also significantly lower in the sepsis nonsurvivors compared with CINS (p<0.05) and sepsis survivors (p<0.01) (FIG. 17D). These findings are consistent with an early and severe adaptive immune suppression in patients with sepsis who ultimately succumb. Septic patient survivors and CINS had a non-statistically significant trend toward more IFN-γ SFU/μl compared with healthy volunteers, and septic survivors had a 3-fold increase in activated T cells per lymphocyte compared with healthy controls (37±8 versus 12±2; p<0.01), as well as a doubling of the total intensity per microliter from 16±3 to 32±13, indicating that a subset of patients with sepsis had an appropriately activated adaptive immune response to infection.

Suppressed ELISpot TNF-α Production is Associated with Sepsis Mortality

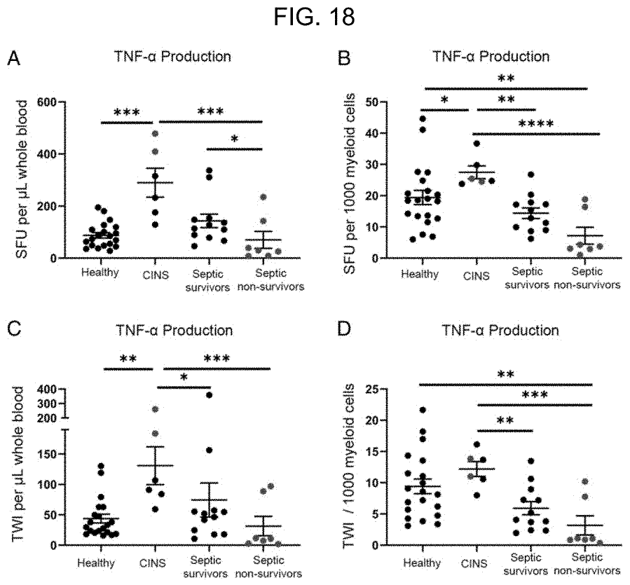

FIG. 18 represents whole blood LPS-stimulated TNF-α production using the ELISpot assay. Healthy volunteers and CINS patient responses were compared with the initial sepsis time point (24-48 h postdiagnosis). The mean number of TNF-α-producing cells per microliter of whole blood was 88±10 for healthy controls, 290±60 for CINS, 143±27 for septic patients who survived, and 70±35 for septic patients who did not survive (FIG. 18A). CINS patients had the highest mean TNF-α production per microliter and per myeloid cell in terms of the number of cytokine-producing cells as well as total intensity, with a 3-fold increase compared with healthy volunteers (p<0.001, p<0.01, respectively) (FIG. 18A, FIG. 18C). Sepsis nonsurvivors had the lowest mean TNF-α production per microliter and per myeloid cell in terms of both the number of cytokine-producing cells as well as total intensity (FIG. 18). Sepsis nonsurvivors had significantly fewer TNF-α-producing cells per microliter compared with sepsis survivors (70±35 versus 143±27; p<0.05) (FIG. 18A). Although there was no significant difference in terms of spots per 1000 myeloid cells plated, there was a strong trend toward decreased numbers of TNF-α-producing myeloid cells in septic nonsurvivors versus septic survivors (7±3 versus 14±2) (FIG. 18B). Additionally, although TNF-α production per microliter was not statistically different between sepsis nonsurvivors and healthy volunteers, they had significantly suppressed the number of cells and intensity when compared on a per 1000 myeloid cell basis (p<0.01) (FIG. 18D).

IFN-γ ELISpot Responses are Comparable in Whole Blood and PBMCs Preparations

A key goal of the study was to compare ELISpot results in diluted whole blood versus PBMCs obtained after Ficoll gradient separation. Note that the ELISpot results for the septic patient using PBMCs have previously been reported (15) for 15 of the 19 patients and are used for a comparison with the whole blood assay.

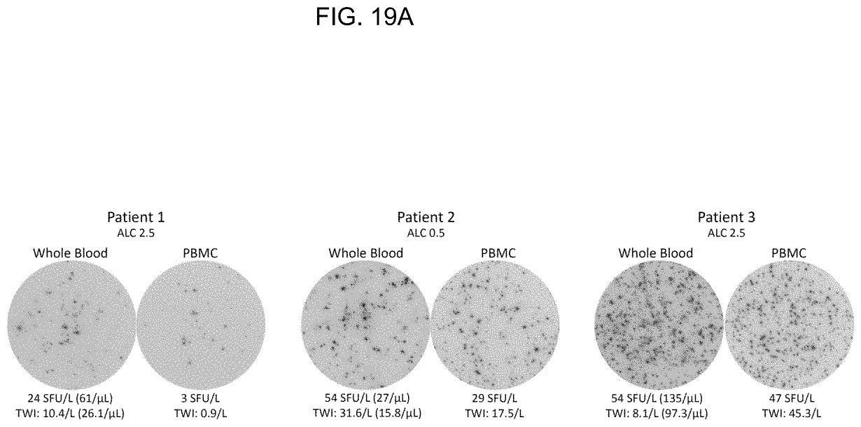

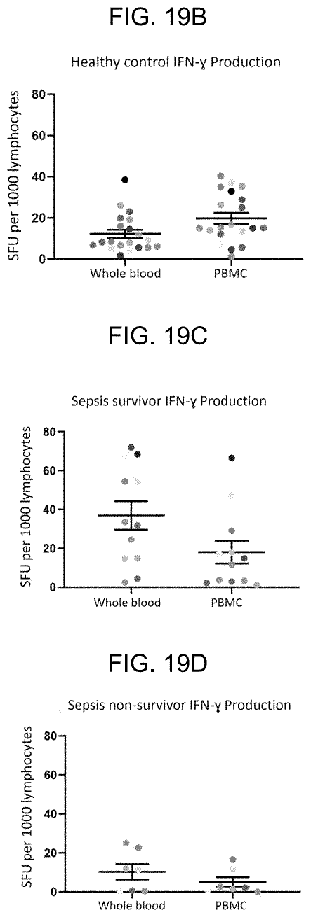

Representative color photomicrographs of IFN-γ ELISpot wells for three septic patients are presented in FIG. 13A, displaying images of single wells for both whole blood and PBMC ELISpot assay methods. Graphs comparing individual whole blood and PBMC IFN-γ production per 1000 lymphocytes for healthy controls, sepsis survivors, and nonsurvivors are depicted in FIG. 19B-FIG. 19D. Means and ranges for IFN-γ production (SFU/1000 lymphocytes) between whole blood and PBMC stimulations were similar in mean values (healthy: 12±2 lymphocytes versus 20±3; septic survivors: 37±8 versus 18±6; sepsis nonsurvivors: 10±4 versus 5±3) and range of variation among each cohort, with PBMCs having a slightly lower number of IFN-γ-producing cells in each cohort except in the healthy group.

Increased TNF-α ELISpot Response in PBMCs Compared with Whole Blood Preparation

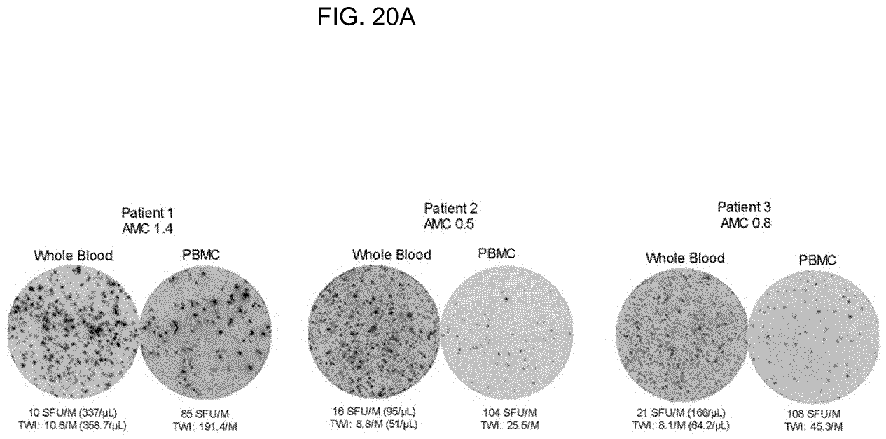

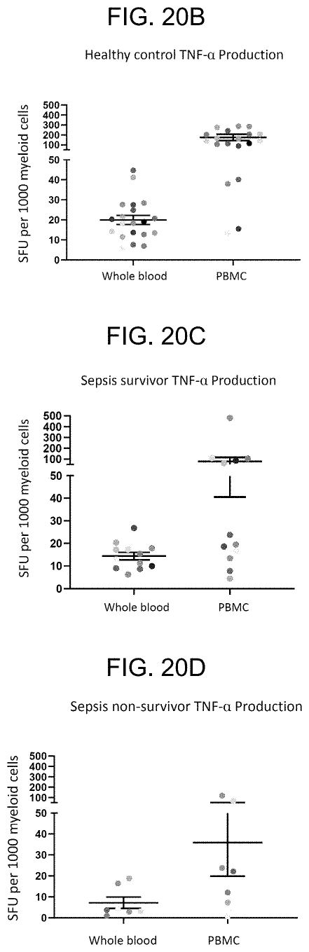

Representative color photomicrographs comparing whole blood versus PBMC ELISpot TNF-α for three septic patients are presented in FIG. 20A. Graphs comparing individual whole blood versus PBMC TNF-α production per 1000 myeloid cells for healthy controls, sepsis survivors, and nonsurvivors are depicted in FIG. 20B-FIG. 20D. To account for neutrophil production of TNF-α, results were therefore normalized to the number of total myeloid cells plated in each experiment. Because whole blood and PBMC factions have vastly different cell type proportions, the corrected results are reported as nearly 10-fold higher for the PBMC assay compared with whole blood. Flow cytometry was performed on the PBMC fraction to determine the contaminating proportion of neutrophils in each sample. The percentage of monocytes in the PBMCs varied from ˜20-30% of total cells and were not statistically different in four cohorts of subjects (i.e., healthy controls, CINS, septic survivors, and septic nonsurvivors) (FIG. 25). In contrast, the percentage of lymphocytes in the PBMCs ranged from ˜50-80% of total cells and was statistically higher in healthy controls versus septic survivors (53±6%) (p<0.01) and sepsis nonsurvivors (50±12%) (p<0.01). Whereas the percentage of neutrophils in the PBMC fraction was 7% or less in healthy controls and CINS, the percentage of neutrophils was ˜15 and 35% in septic survivors and septic nonsurvivors, respectively (p<0.01 for both groups). The results indicate that standard Ficoll gradient separation fails to deplete a significant percentage of neutrophils of the PBMC blood fraction in patients with sepsis. These contaminating low-density neutrophils produce considerable TNF-α (vide infra) and make the comparison between whole blood and PBMC assays more complex.

Whole Blood ELISpot TNF-α Production is Due to Both Neutrophils and Monocytes

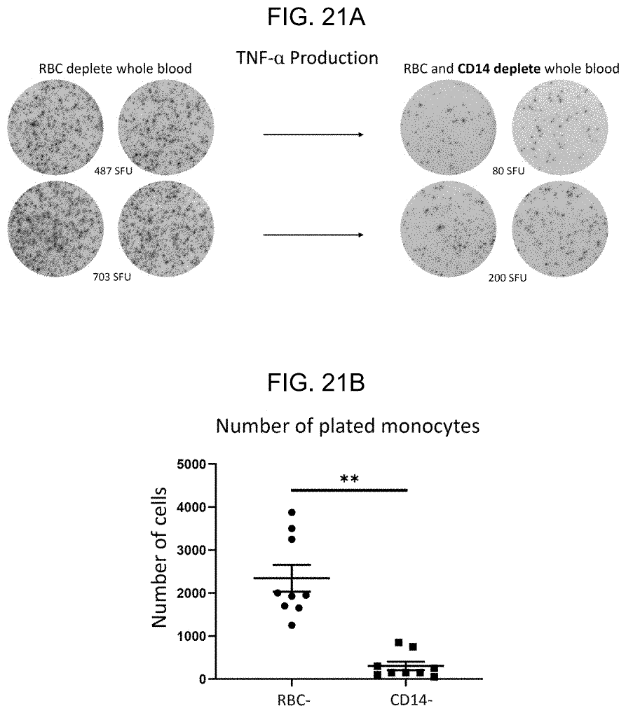

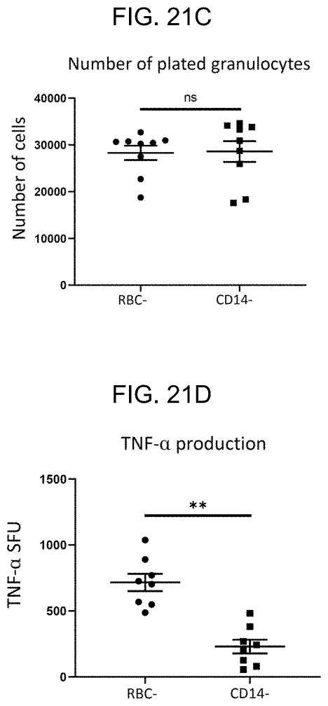

To determine the relative contributions of neutrophils and monocytes to the TNF-α ELISpot production, blood samples from healthy volunteers underwent serial RBC depletion using a magnetic bead erythrocyte depletion kit followed by monocyte magnetic bead depletion using kits from STEMCELL Technologies. Purity of the monocyte depletion was analyzed using flow cytometry for the detection of CD14⁺ cells. Whole blood contained 6.3% monocytes (±0.5%, n=8), RBC-depleted blood contained 4.5% (±0.7%) monocytes, and monocyte-depleted blood contained 0.7% (±0.2%) monocytes. The number of cells positive for TNF-α after overnight LPS stimulation was compared in whole blood versus RBC- and monocyte-depleted blood (FIG. 21). On average, 81%±4.5% (n=8) of the spots in RBC-depleted whole blood were produced by monocytes (range 57-96%) and 27%±2% (n=8) of monocytes plated in each well were activated and secreting TNF-α during the assay (range 20-34%). Put another way, almost 20% of the TNF-α-secreting cells in whole blood ELISpot assays from septic patients are coming from nonmonocyte sources, presumably granulocytes.

The Early and Profound Suppression of Adaptive Immunity is Sustained Throughout Sepsis in Nonsurvivors

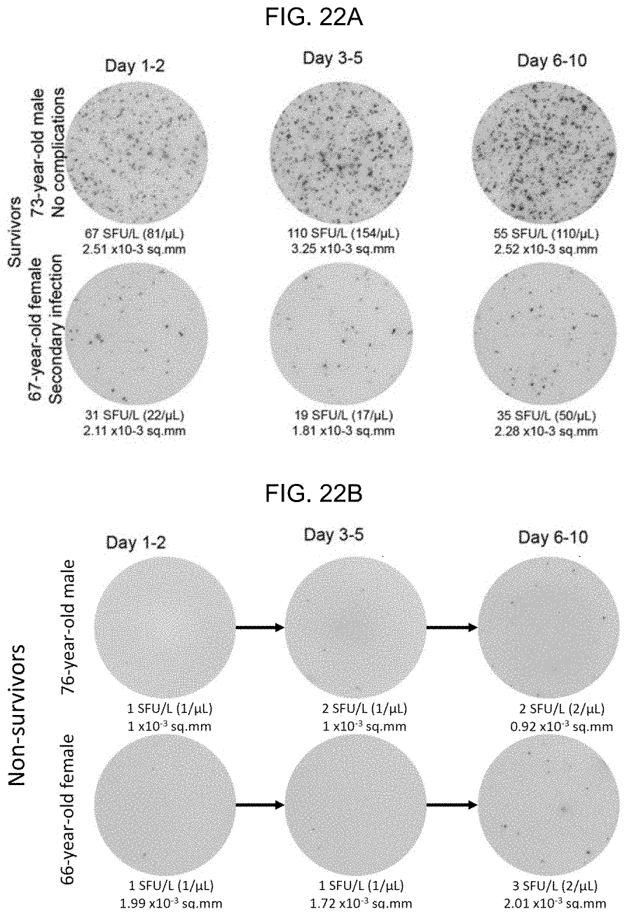

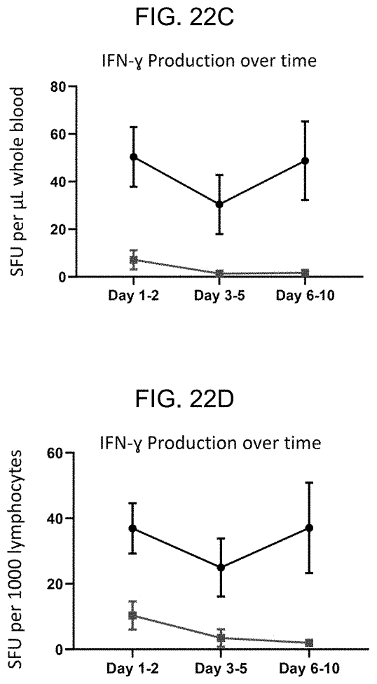

Serial time course examination of septic patient IFN-γ production via ELISpot is presented for patients who survived sepsis versus those who died of sepsis. Representative color micrographs are presented for two sepsis survivors and two nonsurvivors for three consecutive time points (FIG. 22A, FIG. 22B). Data were analyzed by both the number of IFN-γ-producing lymphocytes per 1000 lymphocytes and number of IFN-γ-producing lymphocytes/μl of diluted whole blood. In both cases, septic survivors had increased IFN-γ production compared with nonsurvivors, which was sustained over the course of the 6-10 d after sepsis diagnosis (FIG. 22C, FIG. 22D). This impairment in IFN-γ production that was present on their initial presentation was sustained throughout their entire period of study. There was a non-statistically significant trend toward worsening IFN-γ production as sepsis persisted.

The Early and Profound Suppression in Innate Immunity is Sustained Throughout Sepsis in Nonsurvivors

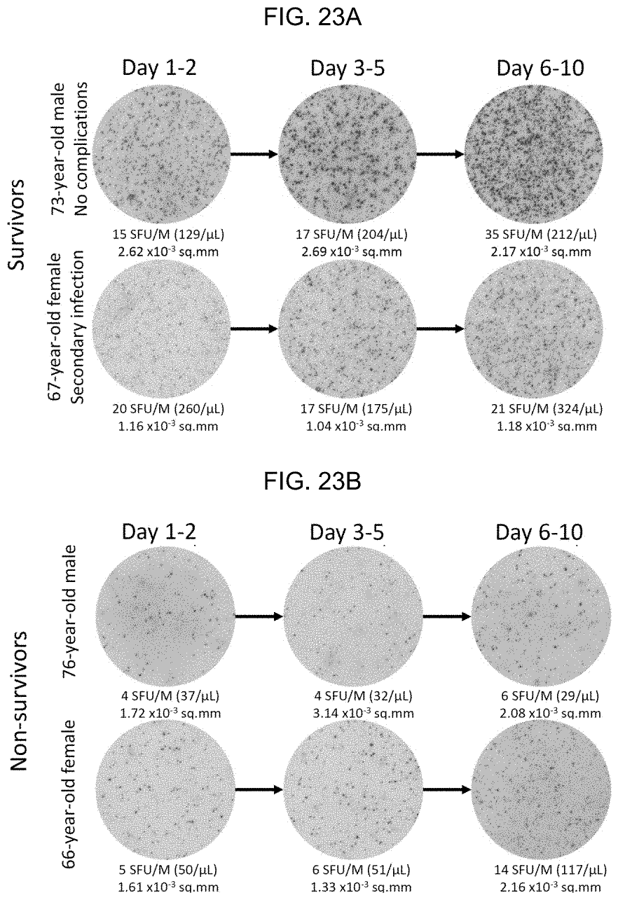

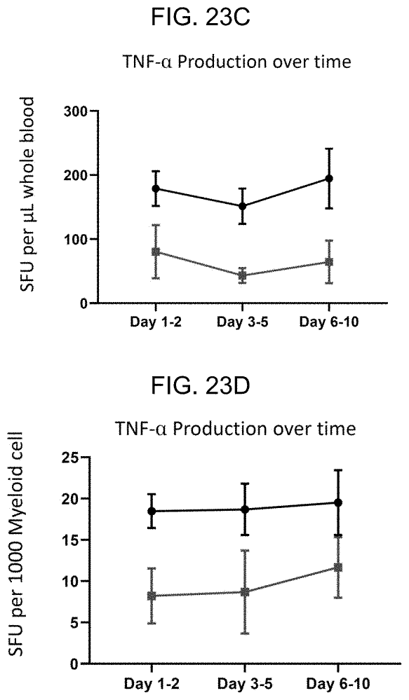

Serial time course examination of septic patient TNF-α production via ELISpot is presented for patients who survived sepsis versus patients who died. Representative color micrographs are presented for two sepsis survivors and two nonsurvivors for three consecutive time points (FIG. 23A, FIG. 23B). The number of TNF-α-producing myeloid cells was increased in septic survivors compared with septic patients who expired and was sustained throughout the study period of up to 10 d following diagnosis of sepsis. Sepsis nonsurvivors demonstrated sustained innate immune dysfunction with a sustained low number of LPS-stimulated cells producing TNF-α (FIG. 23C, FIG. 23D).

IL-7 Restores Adaptive but not Innate Immune Response

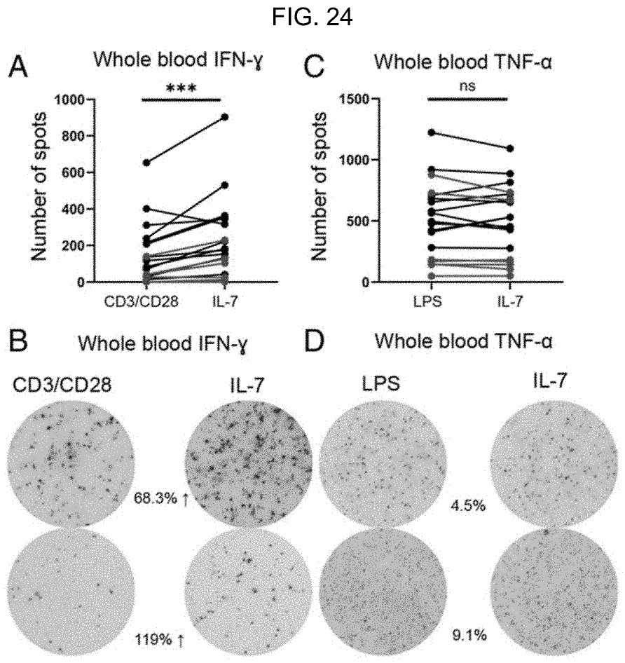

Another key goal of this study was to investigate if the ELISpot assay could be used to determine the potential ex vivo efficacy of immune-adjuvant drug therapies using whole blood ELISpot. In this manner, the potential value of specific immune therapies on cell preparations from individual septic patients could be tested. As proof of principle, we evaluated the ability of IL-7, a potent T cell activator that has undergone phase I/II trials in sepsis, to improve the T cell response in patient samples. Previous work by our group has demonstrated a significant increase in septic patient IFN-γ production using IL-7, and these findings are compared in this study with whole blood ELISpot assay (28). This is a critical comparison as whole blood ELISpot has the potential to be used in ex vivo determination of immunotherapy candidacy in several disease states. Overall, septic patients had a 172%±77 increase in the number of spots when stimulated with IL-7 (p<0.005) (FIG. 24A). This effect was greater in the severely immunosuppressed cohort of patients who did not survive. Survivors had a 102%±32 increase in spot numbers with IL-7, whereas the nonsurvivors had a 312%±240 increase. A similar effect of IL-7 to restore T cell IFN-γ production in septic patients was observed in PBMCs (p<0.0001) (FIG. 18A). In contrast, for whole blood ELISpot, IL-7 does not increase the number of TNF-α-producing cells (FIG. 24C). Surprisingly, for PBMC ELISpot, IL-7 did increase TNF-α production in 13/19 septic patients, resulting in an overall 97% increase in TNF-α SFUs (p<0.01) (FIG. 18B). Similarly, IL-7 significantly increased the SFUs in both healthy subjects and CINS patients in both whole blood and PBMC ELISpot assays (FIG. 18C, FIG. 18E). For TNF-α, there was no change in SFUs for CINS in the whole blood assay, but there was a significant increase using the PBMC assay. For healthy subjects, there was a significant decrease in TNF-α SFUs for whole blood (p<0.01) and no change in PBMCs (FIG. 18D, FIG. 18F).

Circulating Pro- and Anti-Inflammatory Cytokines in Sepsis

Analysis of prototypical pro- and anti-inflammatory cytokines were quantitated using a human cytokine Luminex panel in patients with sepsis (n=15) during their hospital course and compared with values for healthy controls (n=9) and CINS (n=4) patients (TABLE 9) Note that the cytokine data for the healthy control and CINS cohorts, but not the septic patients, have been previously reported (15) and are used in this study to compare with septic patient cytokine response. There was no significant difference in any cytokine concentration between sepsis survivors and non-survivors. Circulating plasma TNF-α levels were significantly higher in septic (p<0.0001) and CINS (p<0.005) patients compared with healthy controls. Elevated IL-6 levels were not associated with any IFN-γ or TNF-α phenotype. Interestingly, plasma TNF-α levels that were above 5 pg/ml were associated with higher unstimulated ex vivo TNF-α production. Surprisingly, IL-8 is inversely related with ex vivo TNF-α production, with the higher levels (>80 pg/ml) being associated with low TNF-α production. Circulating IL-10 levels ranged from 1.5 to 524 pg/ml in septic patients. Although the individual patient with the highest (524 pg/ml) IL-10 level died, the remainder of patients with IL-10 levels >15 pg/ml were associated with a favorable outcome and higher ex vivo IFN-γ production. Of the eight patients with elevated IL-6 levels (>70 pg/ml), there were two mortalities.

TABLE 9
Serum cytokine profile over time in patients with sepsis

| | Septic 1-2 d | Septic 3-6 d | Septic 7-11 d | Healthy Control | CINS^{a} |
|---|---|---|---|---|---|
| | (n = 15), | (n = 13), | (n = 7), | (n = 9), | (n = 4), |
| | Mean ± SEM | Mean ± SEM | Mean ± SEM | Mean ± SEM | Mean ± SEM |
| Cytokine | (range) | (range) | (range) | (range) | (range) |
| IL-1β | 1 ± 0.32 | 1.5 ± 0.7 | 2.2 ± 1.6 | 1.3 ± 0.8 | 0.8 ± 0.3 |
| | (<0.08-3.4) | (0.1-9.2) | (0.08-10.7) | (1-2) | (0.6-1) |
| | | | | | (n = 2) |
| IL-6 | 503.4 ± 96.4 | 536.8 ± 77.9 | 362.7 ± 328.3 | 2 ± 0.3 | 137.1 ± 110.3 |
| | (7.3-4849) | (3.9-4849) | (4.7-2179.1) | (1-3.7) | (21.7-422.3) |
| IL-7 | 7 ± 1.2 | 8.7 ± 1.5 | 7.3 ± 1.8 | 35.8 ± 7.8 | 5.5 ± 0.6 |
| | (2.5-18.5) | (2.8-20.9) | (1.3-11.6) | (5.1-75.6) | (4.1-6.3) |
| IL-8 | 83.5 ± 27.4 | 92.7 ± 38.7 | 71.9 ± 27 | 15 ± 1 | 73 ± 25.8 |
| | (3.9-377.7) | (1.9-455.3) | (3-198.7) | (11.5-17) | (27.4-113.8) |
| IL-10 | 62.8 ± 36.7 | 57 ± 30.3 | 57.2 ± 51.9 | 108.1 ± 78.7 | 25.5 ± 6 |
| | (1.5-524.6) | (1.5-359.72) | (1.3-345.2) | (1-667.3) | (12.5-29.5) |
| IL-12 | 92.7 ± 36.1 | 82.1 ± 31.3 | 107.4 ± 63.2 | 50.4 ± 6.8 | 44.4 ± 15.3 |
| | (6.9-548.6) | (7.2-417.5) | (24.2-450.4) | (31.4-77) | (6.1-65.3) |
| MCP-1 | 502.4 ± 103.8 | 596.5 ± 190.8 | 438.7 ± 124.7 | 486.1 ± 50.2 | 414.8 ± 83 |
| | (71.7-1385.4) | (127.1-2362.3) | (112.8-1000) | (182.1-687.6) | (258.6-595.7) |
| IL-1RA | 383.8 ± 205.3 | 313.8 ± 234.9 | 1082.1 ± 1077.7 | 32 ± 4.3 | 108.5 ± 38.5 |
| | (50.2-2831.9) | (13-3034.2) | (26.3-7067.5) | (18.8-49.9) | (40.5-200.4) |
| IFN-γ | 3.5 ± 2.6 | 1.6 ± 0.6 | 0.7 ± 0.3 | 4.8 ± 0.6 | 9 |
| | (0.4-38.3) | (0.4-8.5) | (0.4-2.2) | (1.5-7) | (n = 1) |
| TNF-α | 4.3 ± 0.7 | 5.2 ± 0.7 | 3.9 ± 1.5 | 1.34 ± 0.2 | 6.6 ± 3.8 |
| | (1.9-12.4) | (2.5-10.1) | (1.1-11.6) | (0.7-1.88) | (2.8-16.5) |
| FGF-basic | 15.6 ± 8.8 | 18.2 ± 11.6 | 35.5 ± 31.3 | 23.3 ± 9.1 | 6.5 ± 2.4 |
| | (2.5-134.5) | (4.1-151.4) | (4.5-209.1) | (0-78.5) | (2.3-12.3) |
| G-CSF/CSF-3 | 224.5 ± 193.2 | 29.4 ± 9.2 | 19.1 ± 7.3 | 32.5 ± 7.6 | 8 ± 2.1 |
| | (6.5-2821.2) | (7.5-103.2) | (6.1-54.5) | (8-80) | (2.7-10.4) |
| IL-13 | 5.1 ± 1.3 | 5.8 ± 1.7 | 3.7 ± 0.9 | 3.2 ± 0.9 | 2.7 ± 0.5 |
| | (0.3-19.7) | (1.7-24.4) | (0.9-5.5) | (0.8-9.2) | (1.9-3.7) |
| RANTES | 1310.2 ± 303.3 | 886.1 ± 158.2 | 1034.7 ± 312.4 | 697.9 ± 64 | N/A |
| | (457.7-2058.7) | (282.4-2058.7) | (350.2-2362.4) | (360.7-888.1) | |
| EOTAXIN | 26.5 ± 4.9 | 28.2 ± 5.5 | 22.9 ± 2.9 | 92.4 ± 28.1 | 57.5 ± 26.4 |
| | (9.2-69.5) | (4.2-67) | (10.4-30.2) | (8.1-255.7) | (15.8-101.4) |
| IL-17A | 3.1 ± 0.6 | 3.1 ± 0.5 | 2.9 ± 1.4 | 5.2 ± 1.9 | 3.2 ± 1 |
| | (0.1-6.4) | (0.1-5.5) | (0.1-9.4) | (0.7-18.9) | (1.3-5.4) |
| MIP-1a | 14.9 ± 10.3 | 19.5 ± 16 | 36.9 ± 38.3 | 5.1 ± 2.6 | 11.97 |
| | (1.4-152.7) | (1.4-203.2) | (1.4-249.9) | (0.3-18.5) | (n = 1) |
| GM-CSF | 2.9 ± 1.3 | 3.2 ± 1.3 | 4.5 ± 1.9 | 9.6 ± 8.4 | 1.6 ± 1 |
| | (0.4-18.2) | (0.8-14.7) | (0.5-11.7) | (0.3-72.6) | (0.9-4.3) |
| MIP-1b | 137.5 ± 58.2 | 158.2 ± 79.8 | 237 ± 195.7 | 88.4 ± 11 | 70.7 ± 18.4 |
| | (19.7-871) | (24.8-1019.9) | (20.6-1323.3) | (65.1-152.7) | (33.2-109.4) |
| IL-15 | 228.5 ± 169.1 | 261.5 ± 204.6 | 601 ± 592.7 | 42 ± 9.7 | 122.8 ± 66.9 |
| | (1.8-2490.4) | (1.8-2602.7) | (9.5-3892.4) | (13.3-88.3) | (12.7-274.5) |
| EGF | 89.7 ± 35.7 | 102.1 ± 50.4 | 193.1 ± 155.9 | 69.1 ± 10.7 | 95.2 ± 27.2 |
| | (3.2-546.1) | (3.2-660.4) | (3.2-1056.4) | (41.1-128.9) | (57.9-159.5) |
| IL-5 | 4 ± 1.5 | 4.2 ± 1.3 | 7.7 ± 4.5 | 2.8 ± 0.6 | 2.2 ± 0.4 |
| | (0.4-22.7) | (1.3-17.1) | (0.8-31.3) | (1.1-7) | (1.3-3.1) |
| HGF | 7710.6 ± 4960.9 | 2265.6 ± 824.6 | 3435 ± 2641.6 | 45 ± 9.4 | 556.9 ± 338.8 |
| | (27-54, 374.5) | (198.6-8177.8) | (59.6-17.727.6) | (22.1-89.2) | (49.4-1325.8) |
| VEGF | 2.9 ± 0.7 | 1.8 ± 0.7 | 3.1 ± 1.4 | 2.1 ± 0.7 | 3.6 ± 1.7 |
| | (0.03-11.2) | (0.03-9.23) | (0.8-9.9) | (0.2-5.7) | (0.7-7.2) |
| IL-1a | 3.3 ± 0.9 | 4.3 ± 1.7 | 7 ± 4.3 | 3.2 ± 1.4 | 9.6 ± 7.7 |
| | (0.7-12.7) | (0.3-22.3) | (0.5-30) | (0.5-12.6) | (1.3-29.5) |
| IL-17F | 170.4 ± 43.7 | 173.3 ± 50 | 278.6 ± 94.2 | 53.5 ± 6.8 | 111.3 ± 51.6 |
| | (53.8-710.9) | (71.2-685.8) | (58.7-641.1) | (37.6-101.6) | (42.1-242.5) |
| IFN-α | 31.4 ± 18.7 | 36.7 ± 24.9 | 69.3 ± 64.2 | 26.7 ± 11.9 | 13.6 ± 5.5 |
| | (3.5-281.1) | (2.5-322.1) | (6.2-425.8) | (7.2-113.6) | (6.4-27.7) |
| IL-9 | 7.4 ± 5.4 | 7.6 ± 5.8 | 11.7 ± 10 | 1.3 ± 0.3 | 3 ± 0.5 |
| | (0.6-79.7) | (0.09-74.3) | (0.4-66.6) | (0.2-3.5) | (2.1-3.7) |
| IL-3 | 33.7 ± 18.4 | 34 ± 17.3 | 38.6 ± 26.2 | 26.4 ± 7.9 | 37.7 ± 23.9 |
| | (0.3-263.9) | (1-183.1) | (0.5-180) | (2.8-73) | (12.3-99.7) |
| IL-2 | 62.6 ± 48.6 | 91.5 ± 77.8 | 227.1 ± 231.9 | 14.8 ± 3.6 | 23.5 ± 16.5 |
| | (3.3-714.3) | (3.9-984.1) | (3-1515.2) | (5.6-33.3) | (5-65.4) |
| IP-10 | 98.1 ± 75.1 | 33.1 ± 12.3 | 16.9 ± 3 | 20.1 ± 3.4 | 24.1 ± 10.8 |
| | (4.1-1110) | (7.4-162.6) | (6.5-25.1) | (5.7-33.9) | (5.7-48.6) |
| IL-2R | 197.5 ± 60.1 | 147.7 ± 43.2 | 178.6 ± 49.5 | 24.4 ± 6.2 | 275.9 ± 236.6 |
| | (19.7-829.2) | (21.1-462.1) | (80.8-429.9) | (2.5-51.7) | (39.5-889.5) |
| IL-22 | 74.6 ± 23.7 | 76.4 ± 24.1 | 71.5 ± 23 | 52.2 ± 14 | 38.8 ± 18.4 |
| | (14.8-350.2) | (20.3-321.7) | (17.3-167) | (20.4-136.1) | (14.5-84) |
| MIG | 65.3 ± 18.6 | 63.6 ± 12.4 | 69.4 ± 24.9 | 15.5 ± 2.9 | 24.3 ± 3.3 |
| | (7.8-231.4) | (7.8-141.2) | (8.8-147.5) | (5.4-27.1) | (10-45.5) |
| IL-4 | 4.2 ± 0.7 | 3.9 ± 0.6 | 2.7 ± 0.8 | 3.5 ± 0.6 | 3.3 ± 2.9 |
| | (0.8-9) | (0.8-7.5) | (0.5-5.7) | (1.5-6.6) | (0.3-8) |
| | | | | | (n = 3) |

Discussion

Sepsis remains a major cause of death and has been remarkably resistant to any new therapies (1-5). Undoubtedly, a key problem in developing immunomodulatory therapies for sepsis is the difficulty in evaluating the immunologic status of the individual patient. The functional state of patients' immune systems during sepsis is complex (6-9). Currently, there is an enormous effort underway to develop methods to immune phenotype patients with sepsis. Knowledge of the status of patients' immunity could guide the administration of effective new immune-based therapies that can either dampen damaging cytokine-mediated inflammation or restore immune function in patients who are profoundly immune suppressed.

The present study demonstrates a major advance in the ability to immune phenotype patients. The whole blood ELISpot assay is an effective method to quantify the functional state of patient adaptive and innate cellular function with excellent dynamic range. Circulating peripheral blood, which combines RBCs, WBCs, platelets, cytokines, and chemokines, is considered in combination to be a vital functional organ. In this sense, it is highly informative to measure ex vivo cytokine production as a response to external stimuli in patient samples. Additionally, circulating chemokines and cytokines in the blood plasma fraction from patients with sepsis has potent immunologic effects on the function of the circulating WBCs (32), and removal through PBMC fractionation can dramatically change cellular response to stimuli or therapeutic molecules. Thus, studies testing diluted whole blood are more likely to reflect the in vivo state. Reporting this response per volume of blood is fundamentally comparable between individual patients and offers more practical applicability than absolute cell counts. Finally, use of diluted whole blood has significant technical advantages of reduced preparation time and effort as well as avoiding potential biologic changes to fragile cells from patients with sepsis because of Ficoll gradient separation or any other processing and handling of the sample.

Findings from the current study demonstrate variability and heterogeneity in the innate and adaptive immune response to sepsis. Many septic patients had increased immune activation, indicated by increased IFN-γ and TNF-α production compared with healthy controls, whereas other septic patients were nearly incapable of cell cytokine production (FIG. 17, FIG. 18). Importantly, the whole blood ELISpot results provide insight into a major driving force for mortality in sepsis. Septic patients who died had early, severe, and sustained suppression of adaptive immunity, as measured by ex vivo IFN-γ production. A significant percentage of the nonsurvivors had marked suppression of innate immunity as well. There was a reduction not only in the number of immune effector cells producing key cytokines but also in the amount of cytokines produced by each cell as measured by spot intensity (FIG. 17, FIG. 18). These results are consistent with the contention that immunosuppression is a key pathophysiologic process in sepsis, and therapies that may restore immunity in vivo could be beneficial on patient outcomes (2-4).

Findings in our study using the whole blood ELISpot also revealed a subgroup of septic patients who had an increase in IFN-γ and TNF-α production compared with healthy control subjects (FIG. 17). This subset of septic patients likely comprises patients who are either mounting an appropriate robust immunologic response to the invading pathogens or, if excessively elevated, a damaging exaggerated proinflammatory response. Findings of an elevated adaptive ex vivo IFN-γ response could be perceived as surprising given the numerous previous observations that sepsis induces significant impairment of adaptive immunity. We speculate that there are several reasons for this observation. First, it is likely that many of the lymphocytes from septic and CINS patients had been primed (because of the infection or injury) prior to being plated in the ELISpot wells. Thus, these lymphocytes were ready to produce IFN-γ upon costimulation during the 18-22-h incubation (33). Conversely, the lymphocytes from healthy control subjects were not primed prior to being plated in the ELISpot wells and therefore did not respond as rapidly to stimulation. Second, it is likely, given the differential propensity of lymphocyte subsets to undergo sepsis-induced apoptosis, that the types of circulating lymphocytes (i.e., naive, effector memory, central memory, etc.) are different in patients with sepsis versus healthy control subjects (34). Naive lymphocytes are slower than effector memory cells to respond to stimulation as they first require differentiation before producing cytokines (35). Finally, it is also important to note that patients with sepsis-induced immunosuppression have significant depletion of CD4⁺ and CD8⁺ T cells in spleen, gastrointestinal lymphoid-associated tissues, and secondary lymphoid organs, which is a major cause of sepsis-induced impaired immunity (34).

Another key finding in our study is the high level of spontaneous TNF-α production in the unstimulated patient samples (FIG. 16D). This result could be useful in identifying septic patients who exhibit a proinflammatory phenotype. Differential TNF-α responses to LPS could also serve as an important indicator of immune system exhaustion (36). Our team is currently performing ELISpot assays on additional patients to define the level of boundaries of an appropriate versus an excessive proinflammatory response and to build prognostic models.

The role played by neutrophils in the global immune response and their specific response in the setting of sepsis further highlights the importance of performing the ELISpot assay using whole blood. Neutrophils make large amounts of both pro- and anti-inflammatory cytokines, including TNF-α and IL-10 (37). Results from healthy control subjects showed that ˜20% of the TNF-α produced in the diluted whole blood ELISpot assay is derived from neutrophils. A significant amount of TNF-α that is present in blood from septic patients is likely to have been derived from neutrophils because of the neutrophilia that occurs in sepsis. Neutrophils also express multiple negative costimulatory molecules including programmed cell death 1 (PD-1) and PD-1 ligand (PD-L1) that can suppress T cell function (38). Thus, the results from ELISpot studies using diluted whole blood are much more likely to reflect the actual state of the patient's immune status compared with neutrophil-depleted PBMCs.

Another significant benefit of the ELISpot assay is that not only can it identify patients with sepsis who are at high risk of dying because of immunosuppression, it can also reveal potential immune adjuvant therapies that might effectively reverse the immunosuppression. Importantly, the ELISpot assay can independently assess the functional status of the two major arms of immunity (i.e., adaptive and innate immunity). This ability to discriminate between the effects of sepsis on the two key components of immunity is particularly important given the availability of new immune adjuvants that selectively target key immune effector cell types. There are several immune adjuvants that are undergoing clinical trials in sepsis (e.g., anti-PD-1, anti-PD-L1, GM-CSF, and IL-7) (11-13). In the current study, IL-7 added ex vivo to septic patient samples effectively restored T cell IFN-γ production in the majority of septic patients. By restoring host immunity, IL-7 could potentially accelerate eradication of the primary infection and decrease secondary hospital-acquired infections. Previously, our group reported that anti-PD-1, anti-PD-L1, and OX-40 agonistic Abs are also effective in restoring T cell IFN-γ production in a variable percentage of septic patients using a PBMC ELISpot assay (28). Thus, the ELISpot assay could be used to identify the optimal immune therapy for use in individual septic patients. This method undoubtedly holds translatable potential to many other fields within critical care, oncology, and autoimmune disease.

Although the ELISpot assay can quantitate numerous cytokines, we elected to examine IFN-γ in the current study for several reasons. T cell exhaustion is a key pathophysiologic mechanism of sepsis-induced immunosuppression and decreased T cell production of IFN-γ is the hallmark of exhausted T cells (39, 40). Furthermore, IFN-γ plays a critical role in host defense against invading pathogens by activating monocytes and macrophages to eliminate invading microbes. Decreased IFN-γ production correlates with worsened survival in animal models of sepsis (41), and administration of IFN-γ showed clinically beneficial effects on infectious outcomes in patients with sepsis and trauma (26, 42). Because of the overabundance of IFN-γ receptors that are present on virtually all nucleated cells, circulating levels of IFN-γ are typically either minimally elevated or not above baseline detection in patients with sepsis. Thus, the ELISpot assay for IFN-γ production is the ideal method to evaluate adaptive immune function and assess immune-adjuvant therapies that impact IFN-γ production because of its exquisite sensitivity and the inability to follow IFN-γ blood levels. Although IFN-γ plays a central role in host antimicrobial defenses, it will be important to define the impact of sepsis on T cell-stimulated production of other cytokines (e.g., IL-2 and TNF-α) that are also critical for a coordinated response to invading pathogens. Similarly, ELISpot assay of monocyte production of additional cytokines such as IL-6 and IL-12 will provide important mechanistic insights into sepsis-induced immunosuppression.

In this regard, the LPS-stimulated whole blood TNF-α release assay developed by Hall et al. (43) has been useful in identifying pediatric patients with sepsis or influenza who have impaired immunity and are more likely to have an increased prevalence of secondary infections and death (44). Although this LPS-stimulated whole blood method has been useful in pediatric patients, it has not yet been shown to have similar utility in adult patients with sepsis and lacks a readout of the adaptive immune response. Additionally, the ELISpot assay has the ability to isolate more discrete immune-suppressive phenotypes by determining the number of cells producing the desired cytokine and can differentiate between low and high cytokine-producing cells using the total area and intensity of each spot (45).

Sepsis is a heterogeneous disorder, and whole blood ELISpot on a larger cohort of patients to prospectively predict outcome and potential responsiveness to therapeutic interventions studies currently are underway. The present study related the ELISpot immunologic findings to the key end point of hospital survival. Future studies will correlate the ELISpot assays to additional clinical metrics that reflect the integrity of the patients' immunity. IFN-γ and TNF-α whole blood ELISpot results will be correlated, for example, with the prevalence of secondary hospital-acquired infections, duration of sepsis, and hospital readmissions. Finally, evaluating whole blood ELISpot data in patients with sepsis will be performed because of a variety of diverse bacterial and fungal pathogens that may have unique effects on host immunity.

In conclusion, there is significant heterogeneity in the immune response in patients with sepsis. Whereas some septic patients have increased IFN-γ and TNF-α production compared with healthy volunteers, many septic patients have severe suppression of immunity. Septic patients who died had early, severe, and sustained immune suppression, as indicated both by a decrease in the number of cytokine-producing immune effector cells and a decrease in the amount of cytokine produced on a per cell basis. Performing the ELISpot assay in patient-diluted whole blood is feasible, easy to perform, and likely to reflect the actual clinical state of the patient's immunity. The whole blood ELISpot assay offers a significant advance in the ability to immune phenotype patients with sepsis and to guide therapy of new potential immune adjuvants that are currently being tested in the treatment of sepsis. For example, administration of corticosteroids in patients with septic shock might be guided by ELISpot analysis of T cell function. Patients in septic shock who have severe depression of T cell function on ELISpot assay might not be good candidates for corticosteroids, which could further exacerbate the T cell depression. The whole blood ELISpot assay may have broad clinical applicability in guiding immune therapies in many disorders, including patients with autoimmunity and cancer and patients who have undergone organ transplantation.

### REFERENCES

1. Singer M, Deutschman C S, Seymour C W, Shankar-Hari M, Annane D, Bauer M, Bellomo R, Bernard G R, Chiche J D, Coopersmith C M, et al. 2016. The Third International Consensus definitions for sepsis and septic shock (Sepsis-3). JAMA 315: 801-810.
2. Venet F, and Monneret G. 2018. Advances in the understanding and treatment of sepsis-induced immunosuppression. Nat. Rev. Nephrol 14: 121-137.
3. Angus D C, and van der Poll T. 2013. Severe sepsis and septic shock. N. Engl. J. Med 369: 2063.
4. Hawkins R B, Raymond S L, Stortz J A, Horiguchi H, Brakenridge S C, Gardner A, Efron P A, Bihorac A, Segal M, Moore F A, and Moldawer L L. 2018. Chronic critical illness and the persistent inflammation, immunosuppression, and catabolism syndrome. Front. Immunol 9: 1511.
5. Martin M D, Badovinac V P, and Griffith T S. 2020. CD4 T cell responses and the sepsis-induced immunoparalysis state. Front. Immunol 11: 1364.
6. Danahy D B, Kurup S P, Winborn C S, Jensen I J, Harty J T, Griffith T S, and Badovinac V P. 2019. Sepsis-induced state of immunoparalysis is defined by diminished CD8 T cell-mediated antitumor immunity. J. Immunol 203: 725-735.
7. Wakeley M E, Gray C C, Monaghan S F, Heffernan D S, and Ayala A. 2020. Check point inhibitors and their role in immunosuppression in sepsis. Crit. Care Clin 36: 69-88.
8. Hotchkiss R S, and Sherwood E R. 2015. Immunology. Getting sepsis therapy right. Science 347: 1201-1202.
9. Hotchkiss R S, Monneret G, and Payen D. 2013. Sepsis-induced immunosuppression: from cellular dysfunctions to immunotherapy. Nat. Rev. Immunol 13: 862-874.
10. Hutchins N A, Unsinger J, Hotchkiss R S, and Ayala A. 2014. The new normal: immunomodulatory agents against sepsis immune suppression. Trends Mol. Med 20: 224-233.
11. Francois B, Jeannet R, Daix T, Walton A H, Shotwell M S, Unsinger J, Monneret G, Rimmelé T, Blood T, Morre M, et al. 2018. Interleukin-7 restores lymphocytes in septic shock: the IRIS-7 randomized clinical trial. JCI Insight 3: e98960.
12. Hotchkiss R S, Colston E, Yende S, Angus D C, Moldawer L L, Crouser E D, Martin G S, Coopersmith C M, Brakenridge S, Mayr F B, et al. 2019. Immune checkpoint inhibition in sepsis: a phase 1b randomized, placebo-controlled, single ascending dose study of antiprogrammed cell deathligand 1 antibody (BMS-936559). Crit. Care Med 47: 632-642.
13. Hotchkiss R S, Colston E, Yende S, Crouser E D, Martin G S, Albertson T, Bartz R R, Brakenridge S C, Delano M J, Park P K, et al. 2019. Immune checkpoint inhibition in sepsis: a Phase 1b randomized study to evaluate the safety, tolerability, pharmacokinetics, and pharmacodynamics of nivolumab. Intensive Care Med 45: 1360-1371.
14. Remy K E, Brakenridge S C, Francois B, Daix T, Deutschman C S, Monneret G, Jeannet R, Laterre P F, Hotchkiss R S, and Moldawer L L. 2020. Immunotherapies for COVID-19: lessons learned from sepsis. Lancet Respir. Med 8: 946-949.
15. Remy K E, Mazer M, Striker D A, Ellebedy A H, Walton A H, Unsinger J, Blood T M, Mudd P A, Yi D J, Mannion D A, et al. 2020. Severe immunosuppression and nota cytokine storm characterizes COVID-19 infections. JCI Insight 5: e140329.
16. Pierrakos C, Velissaris D, Bisdorff M, Marshall J C, and Vincent J L. 2020. Biomarkers of sepsis: time for a reappraisal. Crit. Care 24: 287.
17. Czerkinsky C C, Nilsson L A, Nygren H, Ouchterlony 0, and Tarkowski A. 1983. A solid-phase enzyme-linked immunospot (ELISPOT) assay for enumeration of specific antibody-secreting cells. J. Immunol. Methods 65: 109-121.
18. Sedgwick J D, and Holt P G. 1983. A solid-phase immunoenzymatic technique for the enumeration of specific antibody-secreting cells. J. Immunol. Methods 57: 301-309.
19. Helms T, Boehm B O, Asaad R J, Trezza R P, Lehmann P V, and Tary-Lehmann M. 2000. Direct visualization of cytokine-producing recall antigen-specific CD4 memory T cells in healthy individuals and HIV patients. J. Immunol 164: 3723-3732.
20. Augustine J J, and Hricik D E. 2012. T-cell immune monitoring by the ELISPOT assay for interferon gamma. Clin. Chim. Acta 413: 1359-1363.
21. MObs C, and Schmidt T. 2016. Research techniques made simple: monitoring of T-cell subsets using the ELISPOT assay. J. Invest. Dermatol 136: e55-e59.
22. Cox J H, Ferrari G, and Janetzki S. 2006. Measurement of cytokine release at the single cell level using the ELISPOT assay. Methods 38: 274-282.
23. Li W, Chen W, Herberman R B, Plotnikoff N P, Youkilis G, Griffin N, Wang E, Lu C, and Shan F. 2014. Immunotherapy of cancer via mediation of cytotoxic T lymphocytes by methionine enkephalin (MENK). Cancer Lett 344: 212-222.
24. Patil N K, Bohannon J K, and Sherwood E R. 2016. Immunotherapy: a promising approach to reverse sepsis-induced immunosuppression. Pharmacol. Res 111: 688-702.
25. Hotchkiss R S, and Moldawer L L. 2014. Parallels between cancer and infectious disease. N. Engl. J. Med 371: 380-383.
26. Döcke WD, Randow F, Syrbe U, Krausch D, Asadullah K, Reinke P, Volk H D, and Kox W. 1997. Monocyte deactivation in septic patients: restoration by IFN-gamma treatment. Nat. Med 3: 678-681.
27. Munoz C, Carlet J, Fitting C, Misset B, Blériot JP, and Cavaillon J M. 1991. Dysregulation of in vitro cytokine production by monocytes during sepsis. J. Clin. Invest 88: 1747-1754.
28. Thampy L K, Remy K E, Walton A H, Hong Z, Liu K, Liu R, Yi V, Burnham C D, and Hotchkiss R S. 2018. Restoration of T Cell function in multi-drug resistant bacterial sepsis after interleukin-7, anti-PD-L1, and OX-40 administration. PLoS One 13: e0199497.
29. Mazer M, Unsinger J, Drewry A, Walton A, Osborne D, Blood T, Hotchkiss R, and Remy K E. 2019. IL-10 has differential effects on the innate and adaptive immune systems of septic patients. J. Immunol 203: 2088-2099.
30. Ferrando-Martinez S, Huang K, Bennett A S, Sterba P, Yu L, Suzich J A, Janssen H L A, and Robbins S H. 2019. HBeAg seroconversion is associated with a more effective PD-L1 blockade during chronic hepatitis B infection. JHEP Rep 1: 170-178.
31. Benjamini Y K A, and Yekutieli D. 2006. Adaptive linear step-up procedures that control the false discovery rate. Biometrika 93: 491-507.
32. Gentile L F, Cuenca A G, Vanzant E L, Efron P A, McKinley B, Moore F, and Moldawer L L. 2013. Is there value in plasma cytokine measurements in patients with severe trauma and sepsis? Methods 61: 3-9.
33. Schlingmann T R, Shive C L, Targoni O S, Tary-Lehmann M, and Lehmann P V. 2009. Increased per cell IFN-gamma productivity indicates recent in vivo activation of T cells. Cell. Immunol 258: 131-137.
34. Hotchkiss R S, Swanson P E, Freeman B D, Tinsley K W, Cobb J P, Matuschak G M, Buchman T G, and Karl I E. 1999. Apoptotic cell death in patients with sepsis, shock, and multiple organ dysfunction. Crit. Care Med 27: 1230-1251.
35. Schroder K, Hertzog P J, Ravasi T, and Hume D A. 2004. Interferon-gamma: an overview of signals, mechanisms and functions. J. Leukoc. Biol 75: 163-189.
36. Heagy W, Hansen C, Nieman K, Cohen M, Richardson C, Rodriguez J L, and West M A. 2000. Impaired ex vivo lipopolysaccharide-stimulated whole blood tumor necrosis factor production may identify “septic” intensive care unit patients. Shock 14: 271-276, discussion 276-277.
37. Leliefeld P H, Wessels C M, Leenen L P, Koenderman L, and Pillay J. 2016. The role of neutrophils in immune dysfunction during severe inflammation. Crit. Care 20: 73.
38. Patera A C, Drewry A M, Chang K, Beiter E R, Osborne D, and Hotchkiss R S. 2016. Frontline science: defects in immune function in patients with sepsis are associated with PD-1 or PD-L1 expression and can be restored by antibodies targeting PD-1 or PD-L1. J. Leukoc. Biol 100: 1239-1254.
39. Wherry E J 2011. T cell exhaustion. Nat. Immunol 12: 492-499.
40. Boomer J S, To K, Chang K C, Takasu O, Osborne D F, Walton A H, Bricker T L, Jarman S D II, Kreisel D, Krupnick A S, et al. 2011. Immunosuppression in patients who die of sepsis and multiple organ failure. JAMA 306: 2594-2605.
41. Hotchkiss R S, Chang K C, Grayson M H, Tinsley K W, Dunne B S, Davis C G, Osborne D F, and Karl I E. 2003. Adoptive transfer of apoptotic splenocytes worsens survival, whereas adoptive transfer of necrotic splenocytes improves survival in sepsis. Proc. Natl. Acad. Sci. USA 100: 6724-6729.
42. Dries D J, Jurkovich G J, Maier R V, Clemmer T P, Struve S N, Weigelt J A, Stanford G G, Herr D L, Champion H R, Lewis F R, et al. 1994. Effect of interferon gamma on infection-related death in patients with severe injuries. A randomized, double-blind, placebo-controlled trial. Arch. Surg 129: 1031-1041, discussion 1042.
43. Hall M W, Knatz N L, Vetterly C, Tomarello S, Wewers M D, Volk H D, and Carcillo J A. 2011. Immunoparalysis and nosocomial infection in children with multiple organ dysfunction syndrome. Intensive Care Med 37: 525-532.
44. Muszynski J A, Nofziger R, Greathouse K, Nateri J, Hanson-Huber L, Steele L, Nicol K, Groner J I, Besner G E, Raffel C, et al. 2014. Innate immune function predicts the development of nosocomial infection in critically injured children. Shock 42: 313-321.
45. Beckmann N, Huber F, Hanschen M, St Pierre Schneider B, Nomellini V, and Caldwell C C. 2020. Scald injury-induced T cell dysfunction can Be mitigated by Gr1⁺ cell depletion and blockage of CD47/CD172a signaling. Front. Immunol 11: 876.

### Example 3: IL-7 Immunotherapy in a Nonimmunocompromised Patient with Intractable Fungal Wound Sepsis

Abstract

A nonimmunocompromised patient developed life-threatening soft tissue infection with Trichosporon asahii, Fusarium, and Saksenaea that progressed despite maximum antifungal therapies and aggressive debridement. Interleukin-7 immunotherapy resulted in clinical improvement, fungal clearance, reversal of lymphopenia, and improved T cell function. Immunoadjuvant therapies to boost host immunity may be efficacious in life-threatening fungal infections.

Invasive fungal infections are a growing complication following major traumatic injuries that result in extensive soft tissue damage [1, 2]. The Department of Defense has identified combat wound infections due to invasive fungi as an emerging threat and a high priority [1, 2]. Despite aggressive surgical debridement and antimicrobial therapy that is active against the particular fungal pathogens, many infections progress, with resultant substantial morbidity and/or mortality. Progression of infection despite optimal therapy is consistent with the hypothesis that impaired host immunity may be an important pathophysiologic mechanism that renders the fungus refractory to therapy [3, 4].

Drugs that boost the host immune system are increasingly being tested in various infectious disorders in both immunosuppressed and immunocompetent patients. These immune-adjuvant therapies include interferon (IFN)-γ, checkpoint inhibitors (anti-programmed cell death 1 (anti-PD-1), granulocyte-macrophage colony-stimulating factor (GM-CSF), and interleukin (IL)-7 [5-8]. In some cases, these immune-adjuvant therapies have restored indices of immune function and lead to control of refractory infections [5-8]. Herein, we describe the use of IL-7 in a patient with life-threatening soft tissue necrotizing fungal infection that was refractory to the maximal available therapy.

Patient Case

A previously healthy 21-year-old man presented to the hospital after suffering a high-velocity motorcycle accident. He suffered severe injuries including comminuted pelvic fractures, multiple extremity factures, and a catastrophic degloving injury of the buttocks and perineum with gross wound soilage. He also suffered vascular injuries that required angioembolization of his bilateral iliac arteries for hemorrhage control. On postinjury day 7, he was noted to have a rapidly progressing necrotizing soft tissue infection of his buttocks and perineum. Tissue cultures at that time demonstrated a polymicrobial infection including abundant Acinetobacter spp., abundant Pseudomonas spp. (not Pseudomonas aeruginosa), and moderate Stenotrophomonas maltophila. His wound cultures also grew Trichosporon asahii, Saksenaea spp., and Fusarium spp. The Saksenaea isolate was initially identified using conventional fungal identification methods and the sporulation inducement method for Saksenaea as described by Padhye and Ajello [9]. The isolate was then referred to Mayo Clinic (Rochester, Minn., USA) for a species-level identification, but only a genus level could be determined. The Saksenaea isolate underwent susceptibility testing at the University of Texas Health San Antonio using the Clinical and Laboratory Standards Institute (CLSI) broth dilution antifungal reference method. Drug sensitivities were as follows: amphotericin B<0.03 mcg/mL, posaconazole 0.25 mcg/mL, isavuconazole 1 mcg/mL. The Fusarium was identified by phenotype and microscopy. Trichosporon asahii was identified by matrix-assisted laser desorption/ionization time-of-flight mass spectrometry (MALDI-TOF MS). No sensitivities were performed on Fusarium or Trichosporon asahii.

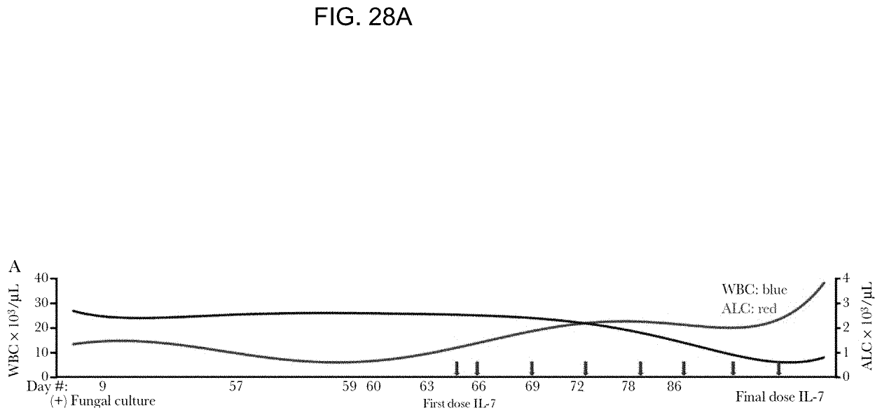

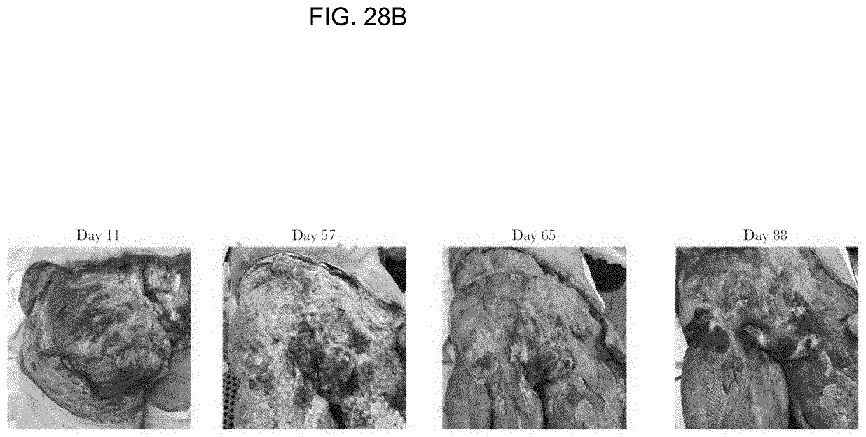

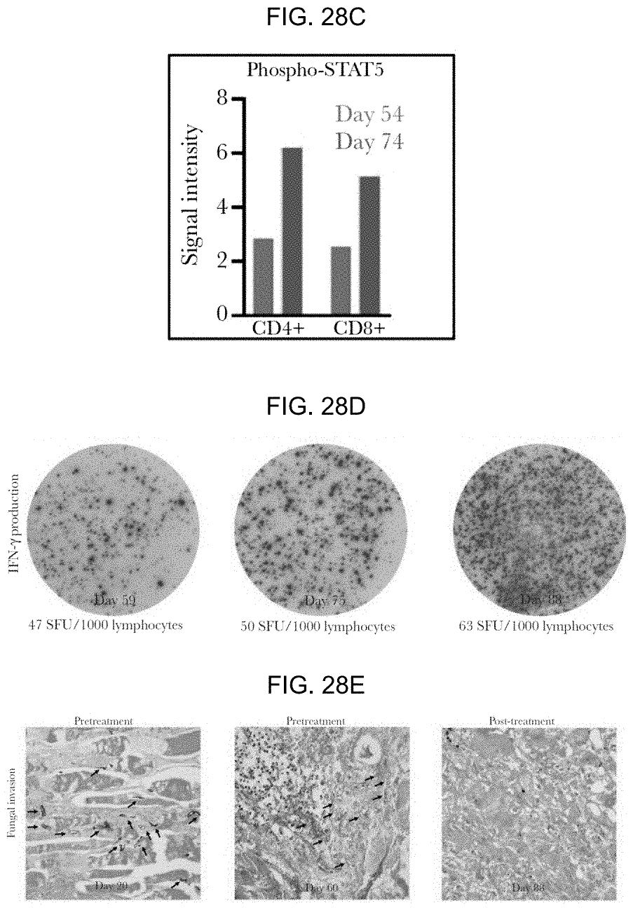

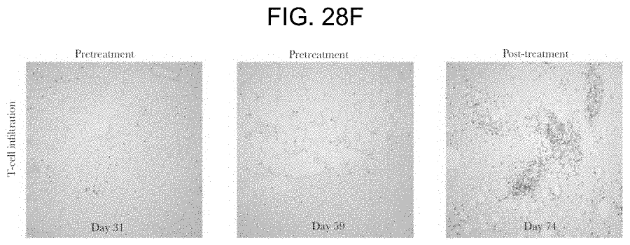

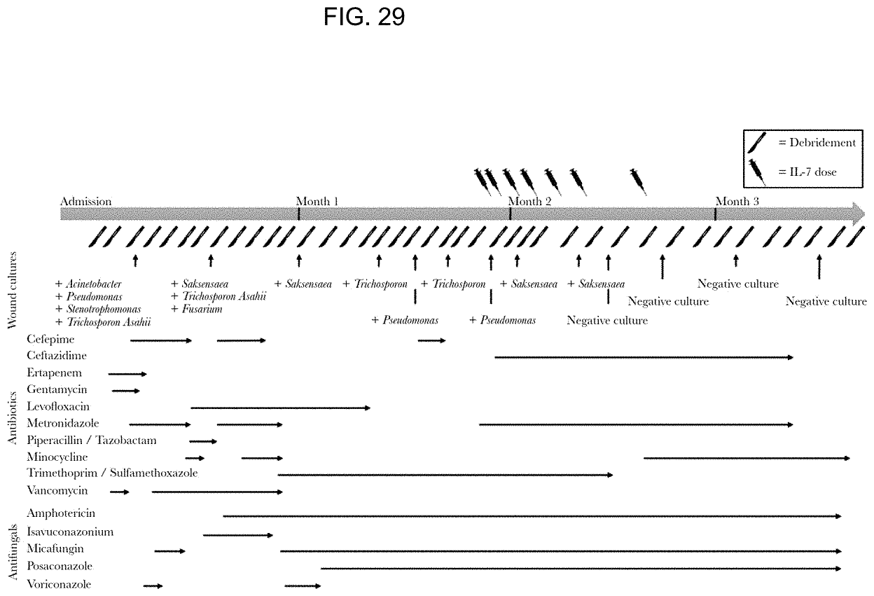

He was aggressively treated with daily or alternating-day operative debridement, broad-spectrum parenteral and topical antibacterial and antifungal therapy. Antimicrobial therapy included ceftazidime, metronidazole, trimethoprim-sulfamethoxazole, micafungin, amphotericin B, and posaconazole (FIG. 29), as well as VERAFLO vac instillation of antimicrobials into the wound bed. The patient's polymicrobial soft tissue infection continued to advance and was accompanied by multisystem organ failure and shock. Over a 4-week period, he required debridement of >3000 cm² of skin, subcutaneous tissue, and muscle involving the entire perineum, bilateral thighs, buttocks, and circumferential abdominal wall (FIG. 28B). Although bacterial pathogens were rapidly eradicated from the patient's wound, cultures were persistently positive for both Trichosporon asahii and Saksenaea species despite triple antifungal therapy comprising posaconazole, amphotericin B, and micafungin. Histopathological exam of wound biopsies showed invasive fungal elements (FIG. 28E).

Despite aggressive antimicrobial and surgical management over the course of 7 weeks after the identification of the polymicrobial infection, his condition continued to worsen. The patient initially had an increased lymphocyte count of up to 2×10³/μL, but soon developed persistent lymphopenia with neutrophilia as high as 50×10³/μL. Therefore, immune-adjuvant therapy with recombinant human IL-7 was considered. IL-7 induces proliferation, maturation, and activation of CD4 and CD8 T cells, which are severely depleted and poorly functional in patients with life-threatening infections including those due to fungal pathogens [6, 7].

Informed consent was obtained from the patient and family, and a test dose of IL-7 (3 μg/kg ideal body weight, intramuscularly; kindly provided by Dr. Michel Morre, RevImmune) was administered on day 59 postinjury, which was well tolerated (FIG. 28A). Twenty-four hours later, the dosage was increased to 10 μg/kg and continued every 3-4 days for 7 doses. The patient's clinical status showed significant improvement beginning at 4-7 days after initiation of IL-7. As illustrated by serial pictures of the wound, the most notable change following initiation of IL-7 was to slow the progression of the invasive soft tissue necrosis (FIG. 28B, green arrows).

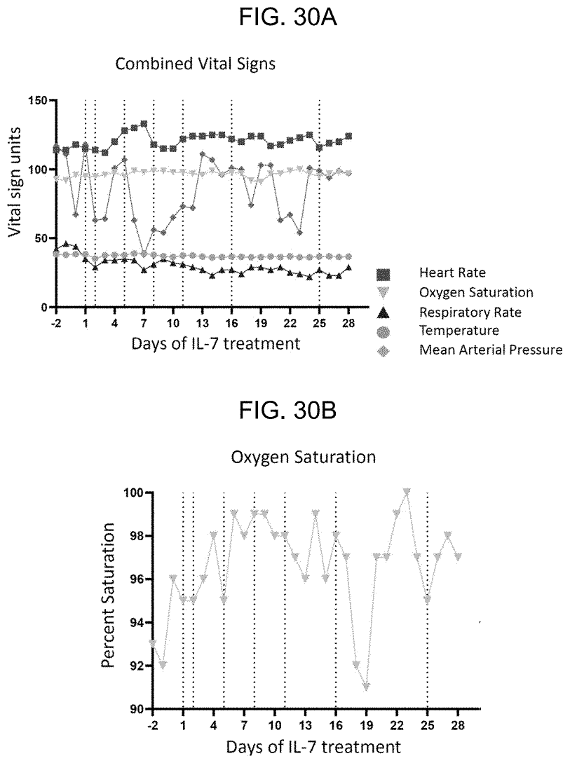

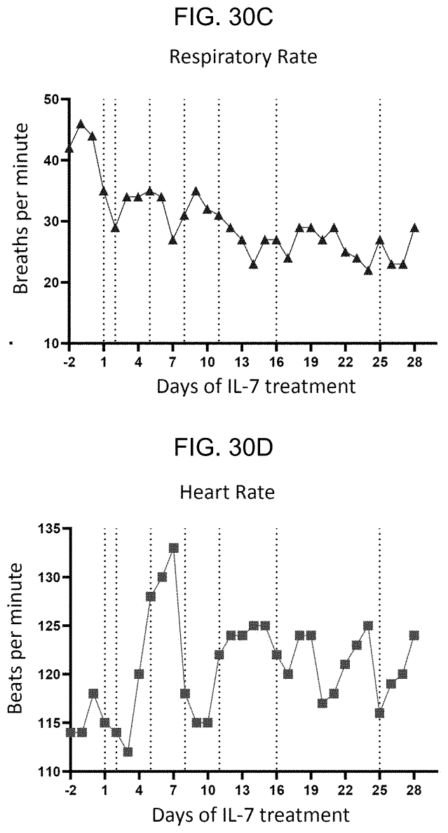

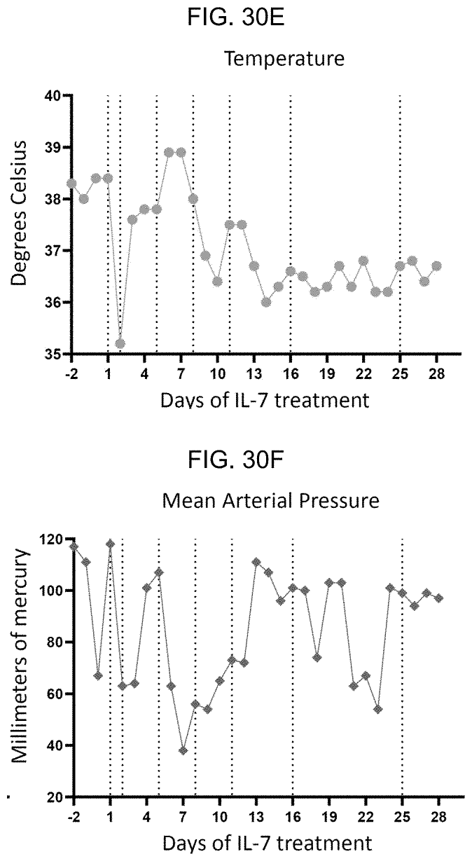

In addition to decreased infectious spread and fungal proliferation, with improved wound healing and healthier-appearing tissue margins, clinical indicators of severity of disease improved within days of IL-7 treatment initiation with improvement of the fever curve, tachycardia, and tachypnea (FIG. 30). Laboratory evidence of resolution of the fungal invasion included progressive decreases in total white blood cell count and increasing absolute lymphocyte counts (FIG. 28A). The patient's absolute lymphocyte count increased >6-fold from a low of 600 lymphocytes/μL to >4000 lymphocytes/μL (upper limit of normal for absolute lymphocyte counts is 3500). Of note, the increase in the patient's absolute lymphocyte count was likely due to both the effect of IL-7 in inducing lymphocyte proliferation and to the resolution of the fungal infection.

The patient had demonstrated persistent fungal growth with rapidly extending tissue borders requiring surgical debridement over the course of 52 days before initiation of IL-7 (TABLE 10). After initiation of IL-7 therapy, an increasing number of tissue cultures did not identify fungal elements, and by the fifth dose of IL-7, the patient had persistently negative cultures. Tissue histology also became negative for fungus at approximately the same time as tissue cultures (FIG. 28E).

After completing IL-7 therapy, the wound beds qualitatively improved with development of healthy granulation tissue facilitating skin grafting. The patient's blood, wound, and bone cultures were negative for bacterial or fungal pathogens for 40 days after completing IL-7 therapy (see TABLE 10 for complete list of cultures). The resolution in wound fungal infection facilitated definitive closure of >90% (>700 cm²) of open wounds. Currently, the patient has a small region of exposed pelvic bone and a persistent perineal wound that remains open due to disruption of his urethra and ongoing urine drainage that is pending reconstruction. A recent biopsy of the exposed pelvic bone and the open wound margin were positive for Trichosporon asahii. This is being treated with an extended course of isavuconazonium. There has been no recurrence of the life-threatening necrotizing fasciitis that resolved with IL-7 therapy.

The effect of IL-7 in terms of improving the patient's T cell function was evaluated by an ex vivo stimulation assay using anti-CD3 and anti-CD28 antibodies (BioLegend, San Diego, Calif., USA) on an IFN-γ ELISPOT assay (Cellular Technologies Limited, Shaker Heights, Ohio, USA), which was performed as previously described [10, 11]. The total number of activated, IFN-γ-producing T cells progressively increased from baseline (before IL-7 therapy), accompanied by a 1.4-fold increase in the proportion of activated T cells (FIG. 28D).

A beneficial effect of IL-7 in infectious disorders is to increase expression of lymphocyte adhesion molecules and induce lymphocyte trafficking to sites of infection [6]. Consequently, immunohistochemical staining using the lymphocyte marker anti-CD3 was performed and demonstrated a marked increase in the number of lymphocytes in the biopsies from the infected wound (FIG. 28F, right-hand panel).

To measure the specific molecular effects underlying the increased T cell responsiveness induced by IL-7, we measured intracellular levels of phospho-STATS (pSTAT5) in CD4 and CD8 T cells using mass cytometry. STATS is the primary T cell differentiation signal downstream of the IL-7 receptor [12]. T cells were identified based on surface marker staining (CD45+/CD15−/CD66b−/CD56−/CD3+). We then measured the pSTAT5 signal intensity. IL-7 was associated with a 3-fold increase in activated STATS in CD4-T cells and a 2-fold increase in CD8-T cells (FIG. 28C). These data provide a putative molecular mechanism for the increase in T cell function induced by IL-7.

Discussion

Critically ill patients with protracted sepsis typically develop profound and persistent immunosuppression [13]. Numerous pathophysiologic mechanisms drive the immune suppression including apoptosis-induced lymphocyte depletion, increased myeloid-derived suppressor cells, and T cell exhaustion. Based upon a growing number of case reports, there is increasing recognition that therapies that boost patient immunity may be beneficial in patients with intractable infections that are nonresponsive to conventional therapies [5, 8, 13]. Particularly relevant to the present case is the use of the immune-adjuvants nivolumab (anti-PD-1) and IFN-γ in a Belgian bomb blast victim who was dying of refractory mucormycosis [5]. Immune-adjuvant therapy resulted in rapid clinical improvement, enhanced immune phenotypic markers, and fungal elimination.

Although a number of immuno-adjuvants are likely to be beneficial, IL-7 is particularly attractive because of its effects on a broad array of immune effector cells including CD4 and CD8 T cells, mucosally associated invariant T cells, and innate lymphoid cells that play key roles in pathogen elimination [8, 13]. Although not presently approved for clinical use, IL-7 is under investigation in multiple clinical trials in infectious and oncologic disorders [6-8]. IL-7 has an excellent safety profile and has been used in >450 patients with both severe infections and various cancers. A double-blind, randomized, phase 2 trial of IL-7 in patients with sepsis showed that IL-7 was well tolerated, reversed sepsis-induced lymphopenia, and enhanced T cell activation [6]. IL-7 has also been shown to prevent lymphocyte apoptosis, improve immune function, and increase survival in a 2-hit animal model of fungal sepsis. IL-7's primary effect is on lymphocytes, but it will have indirect effects to enhance macrophage and neutrophil antimicrobial properties as well. IL-7 increases T cell production of IFN-γ, a potent activator of macrophages. IL-7 also increases T cell IL-17 production, which plays a critical role in fungal infections by enhancing neutrophil migration to sites of infection [6]. IL-7 should be particularly advantageous in patients with profound and persistent lymphopenia because of its potential to prevent lymphocyte apoptosis and induce lymphocyte proliferation.

Although the patient in this report had no history of recurrent fungal infections to suggest an underlying immune deficiency, persistent or recurrent mucocutaneous or invasive fungal infections developing in a “normal” host may be indicative of genetic defects in innate or adaptive immunity [14, 15]. Recently, defects in the caspase recruitment domain containing protein 9 (CARD9) have been reported to occur in patients with severe fungal infections [15].

Recently, immuno-adjuvant therapy to boost host immunity has been proposed as a potential additional powerful weapon in the armamentarium in infectious diseases [8]. The authors believe that the rather remarkable turnaround in the patient's hospital course in the current report provides further support for the concept of augmenting the integrity of the host immune system in life-threatening infections. The ability to evaluate the functional status of the patient's immune system, such as the use of the ELISpot assay in the present case (FIG. 28D), will greatly advance this field by identifying patients who are immunosuppressed and enabling investigators to follow patient response to immuno-adjuvant therapies. Patients with intractable hospital-acquired infections involving multidrug-resistant bacteria or patients with invasive fungal infections are likely good candidates for immuno-adjuvant therapies because they are almost invariably immunosuppressed and have high mortality. Use of IL-7, checkpoint inhibitors, or other immune-adjuvant therapies might be considered on a compassionate basis in patients dying of these intractable fungal infections. If immunotherapy does prove to be an effective new approach in infectious diseases, it could usher in a novel path forward in the battle against increasingly deadly foes.

TABLE 10

| Days post | | | |
|---|---|---|---|
| injury | Test type | Source | Organism |
| Day 9 | Aerobic/anaerobic/ | Tissue, buttocks | Abundant acinetobacter; |
| | gram stain | | Abundant Pseudomonas, not |
| | | | aeruginosa; |
| | | | Mod Stenotrophomonas |
| | | | maltophilia |
| | Mycology culture | Tissue, buttocks | Trichosporon asahii |
| | and stain | | |
| Day 17 | Aerobic/anaerobic/ | Tissue, buttocks | Rare |
| | gram stain | | Trichosporon asahii |
| | Mycology fungal | Left flank | Saksensaea, |
| | | | fusarium species |
| | Mycobacteriology | Tissue, buttocks | Fusarium species; |
| | (AFB) | | Trichosporon asahii |
| | Aerobic/anaerobic/ | Tissue, buttocks, | Mod stenotrophomonas |
| | gram stain | rt thigh | maltophilia |
| | Mycology (fungal) | Left thigh | Saksensaea |
| | Aerobic/anaerobic/ | Right thigh | Trichosporon asahii |
| | gram stain | | |
| | Mycology (fungal) | Right thigh | Saksensaea |
| | Mycobacteriology | Right thigh | Trichosporon asahii |
| | (AFB) | | |
| Day 18 | Mycology (fungal) | Right thigh | Saksensaea |
| Day 20 | Mycology (fungal) | Right thigh | Saksensaea |
| | Aerobic/anaerobic/ | Right thigh | Saksensaea |
| | gram stain | | |
| | Aerobic/anaerobic/ | Left thigh | Saksensaea |
| | gram stain | | |
| | Mycology (fungal) | Left thigh, flank | Saksensaea |
| | Aerobic/anaerobic/ | Left thigh | Saksensaea; |
| | gram stain | | Rare Stenotrophomonas |
| | | | maltophilia |
| | Aerobic/anaerobic/ | Flank | Saksensaea |
| | gram stain | | |
| Day 22 | Aerobic/anaerobic/ | Left buttock | Few Stenotrophomonas |
| | gram stain | | maltophilia |
| Day 30 | Aerobic/anaerobic/ | Lower back | Moderate Stenotrophomonas |
| | gram stain | | maltophilia |
| Day 32 | Aerobic/anaerobic/ | Right groin | Rare Stenotrophomonas |
| | gram stain | | maltophilia |
| | Mycology (fungal) | Right groin | Saksensaea |
| Day 33 | Aerobic/anaerobic/ | Left leg | Few Stenotrophomonas |
| | gram stain | | maltophilia; |
| | | | Saksensaea |
| | Mycology (fungal) | Left leg, left hip | Saksensaea |
| Day 49 | Mycology (fungal) | Abdominal tissue | Trichosporon asahii |
| Day 52 | Aerobic/anaerobic/ | Right thigh | Rare Pseudomonas, not |
| | gram stain | | aeruginosa |
| Day 57 | Aerobic/anaerobic/ | Lower back | Rare Pseudomonas, not |
| | gram stain | | aeruginosa |
| | Mycology (fungal) | Lower back, right | Saksensaea |
| | | thigh | |
| | Aerobic/anaerobic/ | Right thigh | Moderate Pseudomonas, not |
| | gram stain | | aeruginosa; rare mixed |
| | | | microorganisms |
| | Aerobic/anaerobic/ | Abdominal tissue | Few Pseudomonas, not |
| | gram stain | | aeroginosa; mod mixed |
| | | | microorganisms: Saksensaea |
| | Aerobic/anaerobic/ | Left thigh | Moderate Pseudomonas, not |
| | gram stain | | aeruginosa; abundant gram |
| | | | negative bacilli, few gram |
| | | | positive cocci |
| Day 61 | Aerobic/anaerobic/ | Upper back | Rare polymorphonuclear |
| | gram stain | | leukocytes; rare gram pos |
| | | | cocci, rare gram variable |
| | | | bacilli |
| | Aerobic/anaerobic/ | Buttocks | Few gram pos cocci, |
| | gram stain | | Pseudomonas species |
| Day 67 | Mycology (fungal) | Back | Saksensaea |
| | Aerobic/anaerobic/ | Buttocks | Staph epidermidis |
| | gram stain | | |
| Day 73 | Aerobic/anaerobic/ | Right hip | Staph epidenmdis, |
| | gram stain | | Corynebacterium |
| | | | tuberculostearicum |
| | Mycology (fungal) | Right hip, right | Saksenaea |
| | | thigh | |
| Day 76 | Mycology (fungal) | Abdominal Tissue | No growth |
| | | right lower | |
| | | quadrant | |
| | Mycology (fungal) | Back Tissue | No growth |
| | Tissue aerobic and | Abdominal tissue | No growth |
| | anaerobic culture | right lower | |
| | | quadrant | |
| | Tissue aerobic and | Back Tissue | No growth |
| | anaerobic culture | | |
| Day 80 | Mycology (fungal) | Tissue leg, right | No Growth |
| | Mycology (fungal) | Tissue leg, right | No growth |
| | Tissue aerobic and | Tissue leg, right | Few mixed |
| | anaerobic culture | | microorganisms |
| | Tissue aerobic and | Tissue leg, right | No Growth |
| | anaerobic culture | | |
| Day 87 | Mycology (fungal) | Tissue, Flank | No growth |
| Day 118 | Mycology (fungal) | Right Ischium | No growth |
| | | bone graft culture | |
| | Mycology (fungal) | Right Ischium | No Growth |
| | | bone graft culture | |
| | Mycology (fungal) | Left Ischium boss | No Growth |
| | | graft culture | |
| | Mycology (fungal) | Left Ischium bone | No Growth |
| | | graft culture | |
| | Tissue aerobic and | Right Ischium | Moderate Corynebacterium |
| | anaerobic culture | bone graft culture | stratum rare Staphylococcus |
| | | | Epidermidis |
| | Tissue aerobic and | Left Ischium, bone | Moderate Corynebacterium |
| | anaerobic culture | graft culture | striatum |
| Day 138 | Urine | Catheter | Chemically significant plus |
| | | | growth Serratia marcescens |
| | Blood cultures | Blood | Serratia marcescens |
| Day 146 | Tissue aerobic and | Tissue Rt foot | Rare Serratia marcescens |
| | anaerobic culture | | |
| Day 162 | Tissue aerobic and | Wound pelvic | Rare Trichosporon asahii |
| | anaerobic culture | | |
| Day 165 | Mycology | Bone Pelvic | Rare Trichosporon asahii |
| Day 182 | Urine | Catheter | Serratin marcescens |

### REFERENCES

1. Thompson K B, Krispinsky L T, Stark R J. Late immune consequences of combat trauma: a review of trauma-related immune dysfunction and potential therapies. Mil Med Res 2019; 6:11.
2. Kronen R, Liang S Y, Bochicchio G, et al. Invasive fungal infections secondary to traumatic injury. Int J Infect Dis 2017; 62:102-11.
3. Unsinger J, Burnham C A, McDonough J, et al. Interleukin-7 ameliorates immune dysfunction and improves survival in a 2-hit model of fungal sepsis. J Infect Dis 2012; 206:606-16.
4. Spec A, Shindo Y, Burnham C A, et al. T cells from patients with Candida sepsis display a suppressive immunophenotype. Crit Care 2016; 20:15.
5. Grimaldi D, Pradier O, Hotchkiss R S, Vincent J L. Nivolumab plus interferon-γ in the treatment of intractable mucormycosis. Lancet Infect Dis 2017; 17:18.
6. Francois B, Jeannet R, Daix T, et al. Interleukin-7 restores lymphocytes in septic shock: the IRIS-7 randomized clinical trial. JCI Insight 2018; 3:e98960.
7. Hotchkiss R S, Colston E, Yende S, et al. Immune checkpoint inhibition in sepsis: a phase 1 b randomized study to evaluate the safety, tolerability, pharmacokinetics, and pharmacodynamics of nivolumab. Intensive Care Med 2019; 45:1360-71.
8. Hotchkiss R S, Opal S M. Activating immunity to fight a foe—a new path. N Engl J Med 2020; 382:1270-2.
9. Padhye A A, Ajello L. Simple method of inducing sporulation by Apophysomyces elegans and Saksenaea vasiformis. J Clin Microbiol 1988; 26:1861-3.
10. Mazer M B, C Caldwell C, Hanson J, et al. A whole blood enzyme-linked immunospot assay for functional immune endotyping of septic patients. J Immunol 2021; 206:23-36.
11. Remy K E, Mazer M, Striker D A, et al. Severe immunosuppression and not a cytokine storm characterizes COVID-19 infections. JCI Insight 2020; 5:e140329.
12. Pallard C, Stegmann A P, van Kleffens T, et al. Distinct roles of the phosphatidylinositol 3-kinase and STATS pathways in IL-7-mediated development of human thymocyte precursors. Immunity 1999; 10:525-35.
13. Hotchkiss R S, Monneret G, Payen D. Immunosuppression in sepsis: a novel understanding of the disorder and a new therapeutic approach. Lancet Infect Dis 2013; 13:260-8.
14. Lionakis M S. Genetic susceptibility to fungal infections in humans. Curr Fungal Infect Rep 2012; 6:11-22.
15. Vaezi A, Fakhim H, Abtahian Z, et al. Frequency and geographic distribution of CARD9 mutations in patients with severe fungal infections. Front Microbiol 2018; 9:2434.

### Example 4: In Vitro-Administered Dexamethasone Suppresses T Cell Function with Reversal by Interleukin-7 in Coronavirus Disease 2019

Abstract

Objectives: Corticosteroid therapy has become standard of care therapy for hospitalized patients infected with the severe acute respiratory syndrome coronavirus-2 global pandemic-causing virus. Whereas systemic inflammation is a notably important feature in coronavirus disease 2019 pathogenesis, adaptive immune suppression and the inability to eradicate effectively the virus remain significant factors as well. We sought to evaluate the in vitro effects of dexamethasone phosphate on T cell function in peripheral blood mononuclear cells derived from patients with acute, severe, and moderate coronavirus disease 2019. Design: Prospective in vitro laboratory study. Setting: Coronavirus disease 2019-specific medical wards and ICUs at a single-center, quaternary-care academic hospital between Oct. 1, 2020, and Nov. 15, 2020. Patients: Eleven patients diagnosed with coronavirus disease 2019 admitted to either the ICU or hospital coronavirus disease 2019 unit. Three patients had received at least one dose of dexamethasone prior to enrollment. Interventions: Fresh whole blood was collected, and peripheral blood mononuclear cells were immediately isolated and plated onto precoated enzyme-linked immunospot plates for detection of interferon-γ production. Samples were incubated with CD3/CD28 antibodies alone and with three concentrations of dexamethasone. These conditions were also stimulated with recombinant human interleukin-7. Following overnight incubation, the plates were washed and stained for analysis using Cellular Technology Limited ImmunoSpot S6 universal analyzer (ImmunoSpot by Cellular Technology Limited, Cleveland, Ohio). Measurements and main results: Functional cytokine production was assessed by quantitation of cell spot number and total well intensity after calculation for each enzyme-linked immunospot well using the Cellular Technology Limited ImmunoSpot Version 7.0 professional software (CTL Analyzers, Shaker Heights, Ohio). Comparisons were made using t test and using a nonparametric analysis of variance Friedman test. The number of functional T cells producing interferon-γ and the intensity of the response decrease significantly with exposure to 1.2-pg/mL dexamethasone. About 0.12 μg/mL does not significantly affect the functional immune response on enzyme-linked immunospot. Interleukin-7 increases the overall number of activated T cells, including those exposed to dexamethasone. Conclusions: Further evaluation of the effect of immunomodulatory therapies is warranted in coronavirus disease 2019. A refined functional, precision medicine approach that evaluates the cellular immune function of individual patients with coronavirus disease 2019 is needed to better define which therapies could have benefit or cause harm for specific patients.

The ongoing coronavirus disease 2019 (COVID-19) pandemic caused by the severe acute respiratory syndrome coronavirus-2 has resulted in over 2 million deaths worldwide. Although a mechanistic understanding of the disease remains broadly unclear, perturbations in host immunity, injury to respiratory endothelium, and alterations in hemostasis are hallmarks of disease severity. Numerous pharmacologic therapies targeting the viral replication mechanics, inflammatory cascade, compliment system, coagulation cascade, and the host immune response have been tested in clinical trials but demonstrated limited efficacy. No silver bullet to cure critically ill patients and thereby quell the global effects of the pandemic has been revealed; however, corticosteroids have demonstrated improvements in survival, presumably through suppression of a “cytokine storm” and its pathologic effects. Administration of dexamethasone is currently recommended for use in COVID-19 acute respiratory distress syndrome (ARDS) patients by leading authorities (1-3). Administration of dexamethasone is currently recommended for use in COVID-19 ARDS patients by leading authorities (4).

While the anti-inflammatory effects of dexamethasone have potential benefit in reducing cytokine production and edema, corticosteroids suppress a number of critical cellular immune functions that could impair viral clearance and lead to secondary infections (5). Corticosteroids, like dexamethasone, decrease B cell production of immunoglobulins and induce T cell apoptosis, two immune cellular effects that would be counterproductive in COVID-19 (6).

Although cytokine-mediated hyper-inflammation may lead to mortality in COVID-19, many groups, including our own, have demonstrated that COVID-19 is less a disorder of hyper-inflammation than the one characterized by immunosuppression (7-9). Additionally, there exist reports in the literature of corticosteroids in severe COVID-19 patients with ARDS, demonstrating an increased 28-day mortality rate (3). Given the heterogeneity of the immune response in patients with COVID-19 and the potential and deleterious effects of using a glucocorticoid in patients with existing immune suppression, we investigated the effect of dexamethasone on T cell function in blood from hospitalized COVID-19 patients.

Materials and Methods

In a cohort of 11 patients admitted to an academic quaternary care ICU or COVID-19 hospital unit, we obtained the first blood sample within 72 hours from admission. Three of the 11 patients received dexamethasone 6 mg prior to blood sample draw. Two of the patients admitted to the ICU subsequently died. Patient characteristics between ± in vivo dexamethasone are shown in TABLE 11. We evaluated adaptive immune function using the enzyme-linked immunospot (ELISpot) assay to quantitate blood mononuclear cell interferon (IFN)-γ production after CD3/CD28 stimulation from 11 hospitalized COVID-19 patients. We mimicked in vitro dexamethasone administration to the standard 6-mg dexamethasone dose being used in patients with COVID-19.

TABLE 11
Patient Demographics and Clinical Information

| | | |
|---|---|---|
| | Received | Did Not Receive |
| | Dexamethasone | Dexamethasone |
| Demographics | (n = 3) | (n = 8) |

| | | | | |
|---|---|---|---|---|
| Age, mean (range) | 42.6 | (21.6-55.5) | 47.9 | (19.3-69.6) |

Sex, n (%)

| | | | | |
|---|---|---|---|---|
| Female | 0 | (0) | 3 | (37.5) |
| Male | 3 | (100) | 5 | (62.5) |

Race, n (%)

| | | | | |
|---|---|---|---|---|
| African American | 0 | (0) | 7 | (87.5) |
| White | 3 | (100) | 1 | (12.5) |
| Body mass index, | 43.3 | (29-68.7) | 32.3 | (21.7-40.5) |
| mean (range) | | | | |

ICU admission, n (%)

| | | | | |
|---|---|---|---|---|
| Yes | 2 | (66.7) | 3 | (37.5) |
| No | 1 | (33.3) | 5 | (62.5) |

Mortality status, n (%)

| | | | | |
|---|---|---|---|---|
| Alive | 2 | (66.7) | 7 | (87.5) |
| Deceased | 1 | (33.3) | 1 | (12.5) |

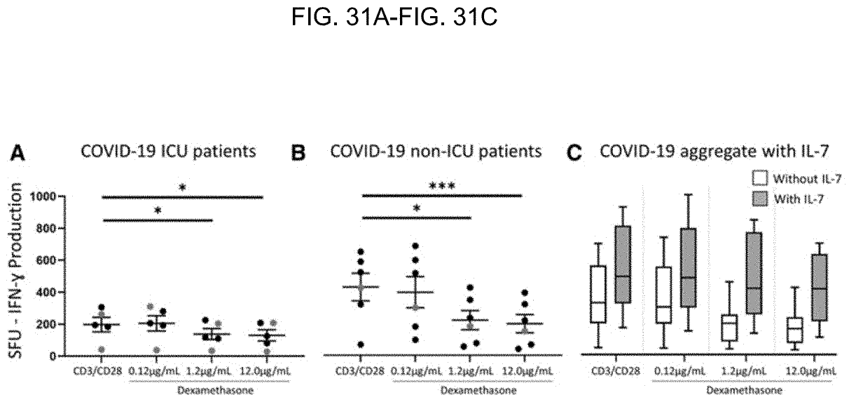

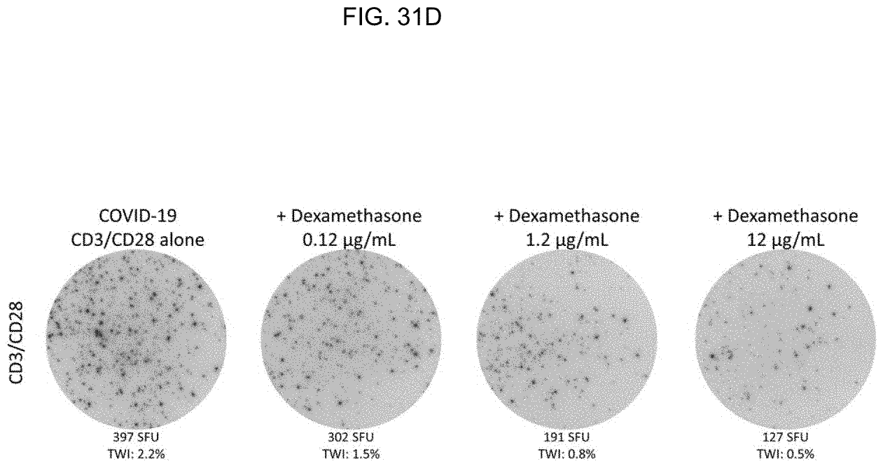

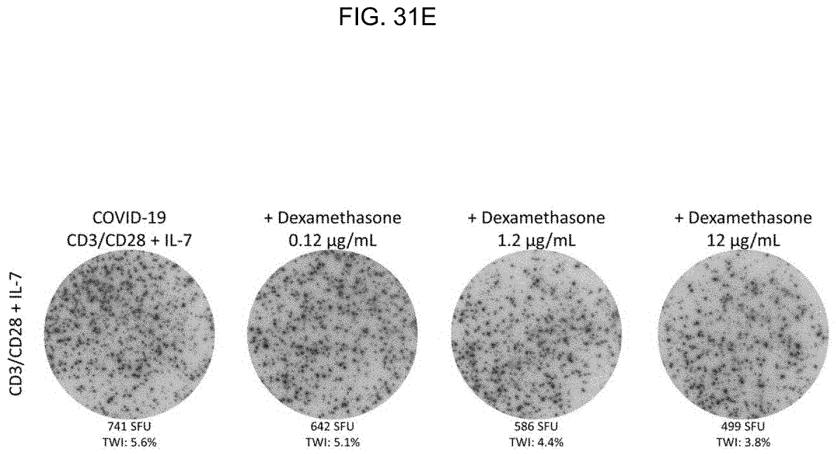

Given this typical daily dose of 6 mg, and an expected peak plasma concentration of approximately 1.5 μg/mL and volume of distribution of 648 mL/kg, dexamethasone concentrations of 0.12, 1.20, and 12.0 μg/mL were tested after CD3/CD28 stimulation in ICU (FIG. 31A) and non-ICU patients (FIG. 31B) (10). Patients that received dexamethasone are shown in green. Representative ELISpot figures are shown in FIG. 31D and FIG. 31E. Samples were compared in ICU or non-ICU patients by analysis of variance (ANOVA) in three separate doses against CD3/CD28 stimulated positive control cells using the GraphPad Prism Version 9.0 software (GraphPad, San Diego, Calif.).

Results

Dexamethasone produced in patients a dose-dependent decrease in T cell IFN-γ production with a 30% (ICU) and 49% (non-ICU) reduction in the number of IFN-γ secreting cells, and 61% (ICU) and 58% (non-ICU), respectively, decrease in IFN-γ production (measured by ELISpot total well intensity), in the 1.20-mg/mL concentration (most closely approximating the 6-mg equivalent in patients) (FIG. 31B, p<0.05; all comparisons). Importantly, when coincubated with both dexamethasone and interleukin (IL)-7, a potent T cell stimulant, T cell function was restored in the aggregate of ICU and non-ICU patients (FIG. 31C). IL-7 has previously been shown to be safely administered and to reverse profound lymphopenia in critically ill patients with COVID-19 and could function as an adjunct to corticosteroid therapy (11).

Discussion

COVID-19 has demonstrated an elusive yet heterogeneous immune phenotype across all patients (7-9). These data make a compelling argument for using a precision medicine approach to the immune endotypes in COVID-19 patients when considering treatments such as corticosteroids. Undeniably, increased severity of illness (ICU vs non-ICU patients) demonstrated, in the absence of corticosteroids, significant immune suppression. However, the effect was dramatically worsened by increasing doses of in vitro administration of dexamethasone, especially in non-ICU, less severe patients. Likewise, IL-7 restoration of T cell IFN-γ production after coincubation with dexamethasone may show a promising therapy for some patients that have T cell exhaustion and concomitant “cytokine storm.”

The strengths of our study include a younger population that may not exhibit immunosenescence as seen with older patients (mean age in this study of 42.6 vs 47.9 yr±dexamethasone), differing severity of illnesses (ICU vs non-ICU), and evaluation of dexamethasone dose response. Nonetheless, our findings (while hypothesis generating) should be taken with caution as they only represent in vitro findings. A before and after T cell IFN-γ production evaluation in patients receiving standard of care dexamethasone would best delineate the true in vivo effects of dexamethasone in this population.

Conclusion

Invariably, application of a therapy such as dexamethasone may be beneficial to some patients and harmful in others. We recommend further evaluation with a refined functional, precision medicine approach that evaluates the cellular immune function of individual patients with COVID-19 to better define which therapies may have benefit or harm. Such an approach may also refine which patients may benefit from other therapies such as tocilizumab, anakinra, or IL-7. Directing therapy at known affected targets of the immune system will undoubtedly improve outcomes in patients and may revitalize therapies that have previously demonstrated a lack of efficacy in large clinical trials. We recommend improved methods to individualize care by assessing the functional state of patient immunity and, thereby, rigorously defining which patients are appropriate to receive immune-modulating therapies to combat this pandemic.

### REFERENCES

1. Sterne J A C, Murthy S, Diaz J V, et al. WHO Rapid Evidence Appraisal for COVID-19 Therapies (REACT) Working Group. Association between administration of systemic corticosteroids and mortality among critically ill patients with COVID-19: A meta-analysis. JAMA. 2020; 324:1330-1341
2. Cano E J, Fuentes X F, Campioli C C, et al. Impact of corticosteroids in coronavirus disease 2019 outcomes: Systematic review and meta-analysis. Chest. 2021; 159:1019-1040
3. Liu J, Zhang S, Dong X, et al. Corticosteroid treatment in severe COVID-19 patients with acute respiratory distress syndrome. J Clin Invest. 2020; 130:6417-6428
4. Health TNlo. NIH COVID-19 Treatment Guidelines—Therapeutic Management. Available at: https://www.covid19treatmentguidelines.nih.gov/therapeutic-management/. Accessed Feb. 22, 2021
5. Coutinho A E, Chapman K E. The anti-inflammatory and immunosuppressive effects of glucocorticoids, recent developments and mechanistic insights. Mol Cell Endocrinol. 2011; 335:2-13
6. Woodward M J, de Boer J, Heidorn S, et al. Tnfaip8 is an essential gene for the regulation of glucocorticoid-mediated apoptosis of thymocytes. Cell Death Differ. 2010; 17:316-323 [PubMed] [Google Scholar]
7. Remy K E, Brakenridge S C, Francois B, et al. Immunotherapies for COVID-19: Lessons learned from sepsis. Lancet Respir Med. 2020; 8:946-949
8. Remy K E, Mazer M, Striker D A, et al. Severe immunosuppression and not a cytokine storm characterizes COVID-19 infections. JCI Insight. 2020; 5
9. Mudd P A, Crawford J C, Turner J S, et al. Distinct inflammatory profiles distinguish COVID-19 from influenza with limited contributions from cytokine storm. Sci Adv. 2020; 6:eabe3024.
10. Perez E M, Rogers L K, Smith C V, et al. Pharmacokinetics of dexamethasone in rats 346. Pediatr Res. 1998; 43:61-61
11. Laterre P F, Francois B, Collienne C, et al. Association of interleukin 7 immunotherapy with lymphocyte counts among patients with severe coronavirus disease 2019 (COVID-19). JAMA Netw Open. 2020; 3:e2016485.

### Example 5: Interleukin-7 Reverses Lymphopenia and Improves T Cell Function in Coronavirus Disease 2019 Patient with Inborn Error of Toll-Like Receptor 3: A Case Report

Abstract

Background: Immunotherapy treatment for coronavirus disease 2019 combined with antiviral therapy and supportive care remains under intense investigation. However, the capacity to distinguish patients who would benefit from immunosuppressive or immune stimulatory therapies remains insufficient. Here, we present a patient with severe coronavirus disease 2019 with a defective immune response, treated successfully with interleukin-7 on compassionate basis with resultant improved adaptive immune function. CASE Summary: A previously healthy 43-year-old male developed severe acute respiratory distress syndrome due to the severe acute respiratory syndrome coronavirus 2 virus with acute hypoxemic respiratory failure and persistent, profound lymphopenia. Functional analysis demonstrated depressed lymphocyte function and few antigen-specific T cells. Interleukin-7 administration resulted in reversal of lymphopenia and improved T cell function. Respiratory function and clinical status rapidly improved, and he was discharged home. Whole exome sequencing identified a deleterious autosomal dominant mutation in TICAM1, associated with a dysfunctional type I interferon antiviral response with increased severity of coronavirus disease 2019 disease. Conclusions: Immunoadjuvant therapies to boost host immunity may be efficacious in life-threatening severe coronavirus disease 2019 infections, particularly by applying a precision medicine approach in selecting patients expressing an immunosuppressive phenotype.

Severe acute respiratory distress syndrome secondary to severe acute respiratory syndrome coronavirus 2 (SARS-CoV-2) carries a ˜50% mortality (¹⁻⁴). Although preferentially affecting individuals of advanced age, younger patients with respiratory failure who require ICU admission and invasive mechanical ventilation maintain a significant risk of mortality. Prospective and retrospective cohorts of patients with coronavirus disease 2019 (COVID-19) have demonstrated mortality rates between 20% and 40% for patients 40-50 years who are admitted to an ICU and approaches 45% if they require intubation (⁵⁻⁷). Severity of disease in COVID-19 is linked to a dysregulated host immune response to the SARS-CoV-2 virus (⁸⁻¹⁰), which has led to the initiation of several clinical trials investigating the use of immunomodulatory agents as adjuvant therapy alongside antiviral medications (^{11,12}). There have been several different immunopathologic mechanisms described in the literature to date, with each potentially benefitting from a distinct targeted therapy. Among these theories, cytokine storm and cellular adaptive immune suppression are the most frequently described in the literature (^{11,13}). One important indicator of cellular adaptive immune dysfunction is lymphopenia. Lymphopenia (<1×10³ cells/μL) is predictive of severity and poor outcome, whereas severe lymphopenia (<0.5×10³ cells/μL) is associated with a 12-fold increased risk of mortality (¹⁴⁻¹⁶). Furthermore, the functional capacity of circulating lymphocytes was assessed using an ex vivo stimulation assay (¹⁷) and is severely impaired in patients with severe COVID-19, as evidenced by defective T cell production of interferon (IFN)-γ after T cell receptor agonist stimulation (¹⁸). Additionally, impairments to immune function and antiviral host defense have been linked to subtle inborn errors of immunity. There are reports that 3.5% of patients with life-threatening COVID-19 pneumonia have deleterious variants in genes involved in type 1 IFN signaling, further supporting therapies that restore immune function (¹⁹⁻²¹).

Herein, we describe the use of interleukin (IL)-7 in a critically ill patient with severe COVID-19 disease, evidence of adaptive immune dysfunction, and the discovery of a genetic defect in Toll-like receptor (TLR)-3.

Case Description

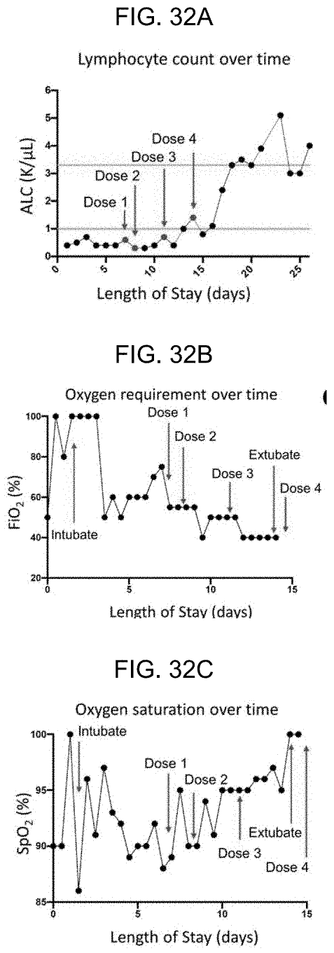

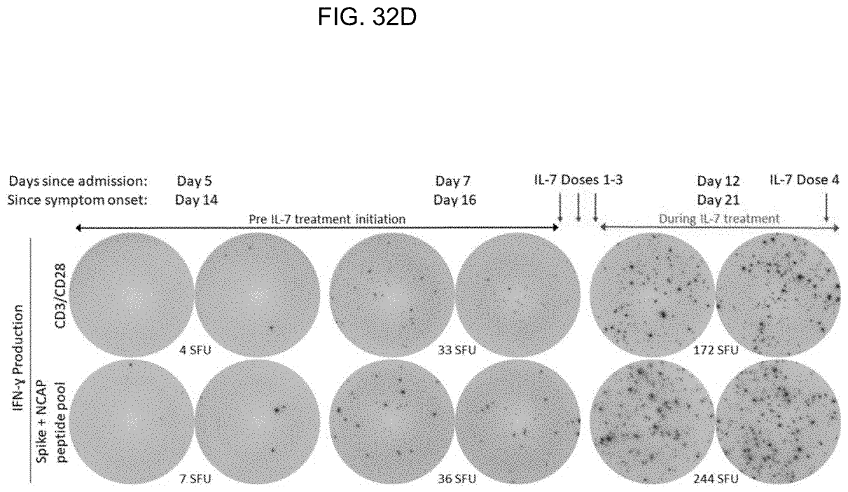

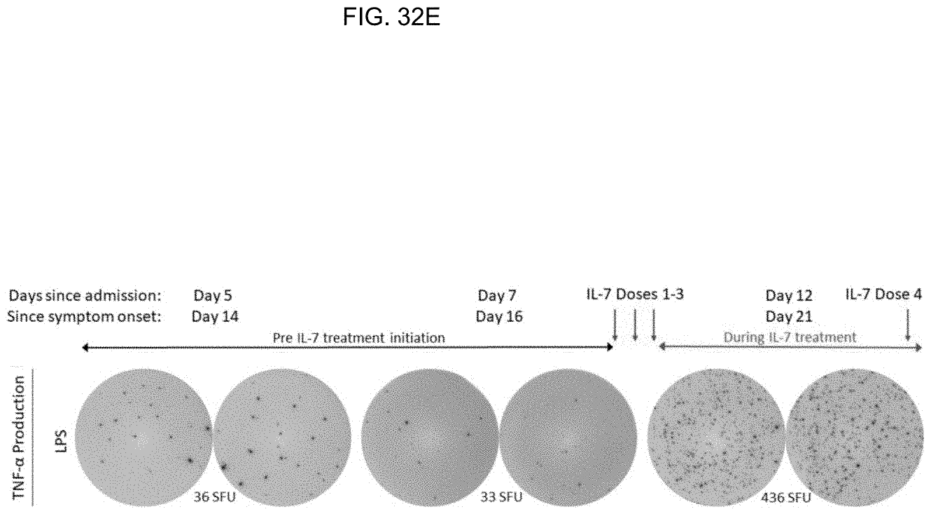

A 43-year-old male with no significant comorbidities presented with severe hypoxemic respiratory failure due to SARS-CoV-2. The patient began experiencing cough, fever, and myalgias 9 days prior to hospitalization and tested positive 2 days after symptom onset. He was prescribed 7 days of dexamethasone, 6 mg daily, at that time by his primary care provider. Despite treatment with high-flow oxygen, convalescent plasma, remdesivir, and continued dexamethasone, the patient developed rapidly worsening hypoxemia necessitating endotracheal intubation and mechanical ventilation within 24 hours of hospitalization. The patient developed refractory hypoxemia requiring 100% Fio₂, high positive end-expiratory pressure (PEEP), and inhaled epoprostenol to maintain adequate oxygen saturations. His absolute lymphocyte count (ALC) was 0.4×10³ cells/μL, and his lymphopenia persisted, ranging from 0.4×10³ to 0.7×10³ cells/μL (FIG. 32A).

Lymphocyte function was assessed by the enzyme-linked immunospot (ELISpot) (Cellular Technology Limited, Shaker Heights, Ohio) assay using ex vivo stimulated peripheral blood mononuclear cells (PBMCs) as previously described (Missouri Baptist Medical Center Institutional Review Board Approval number 1132) (^{18,22,23}). Cellular function is determined by the number of cells secreting cytokines. Cluster of differentiation (CD)-3/CD28 stimulated IFN-γ indicates adaptive immune function, and lipopolysaccharide-stimulated tumor necrosis factor (TNF)-α is used to indicate innate immune function (¹⁸). IFN-γ production in response to SARS-CoV-2 spike glycoprotein and nucleocapsid peptide pool (JPT, Berlin, Germany) is used to determine the patient's antigen-specific T cell response.

On days 5 and 7 post admission, ELISpot showed marked suppression of lymphocyte function (FIG. 32D), and TNF-α production was not elevated, suggesting that the patient did not have an overactive innate response or cytokine storm (FIG. 32E). Additionally, very few IFN-γ-producing SARS-CoV-2-specific T cells were detected (FIG. 32D). One week into hospitalization, the patient remained persistently febrile with continued need for mechanical ventilation requiring 75% Fio₂ with a PEEP of 14 cm H₂O (FIG. 32A-FIG. 32C). He completed 5 days of remdesivir and continued dexamethasone treatment. In light of the patient's failure to improve, the predicted prognosis, and lymphopenia with suppressed lymphocyte function, administration of IL-7 was considered on compassionate basis. Preliminary studies of COVID-19 disease patients treated with IL-7 demonstrated that IL-7 increased ALCs two- to three-fold and was well tolerated (²⁴). Other studies showed that ex vivo addition of IL-7 to PBMCs from critically ill COVID 19 disease patients doubled lymphocyte IFN-γ production (¹⁸).

Given the patient's ex vivo ELISpot response, approval was sought from the Federal Drug Administration (FDA) and obtained via emergency compassionate use authorization (Approved IND number 155018). Consent was obtained, and a test dose of 3 μg/kg of recombinant human IL-7 (RevImmune Inc, Paris, France) was administered via intramuscular injection and was well tolerated (FIG. 32). Twenty-four hours later, 10 μg/kg of IL-7 was administered followed by two additional doses of IL-7 separated by 72 hours.

After initiation of IL-7 therapy, the patient's clinical status steadily improved, and mechanical ventilation was discontinued on day 15 after ICU admission. The patient was discharged home on day 24 of hospitalization (FIG. 32B and FIG. 32C). Paralleling the patient's clinical improvement, his ALC increased ˜10-fold to a maximum of 5.1×10³ lymphocytes/μL (FIG. 32A). Circulating lymphocyte and monocyte function also improved concomitant with initiation of IL-7 treatment as evidenced by multifold increases in the number of IFN-γ-producing T cells and TNF-α-producing cells (FIG. 32D and FIG. 32E). Importantly, given their critical role in viral elimination, the number of SARS-CoV-2 spike protein and nucleocapsid specific T cells improved over 30-fold. Additionally, serial plasma cytokines were obtained and analyzed (Invitrogen ProcartaPlex 9-plex Luminex panel; Thermo Fisher Scientific, Waltham, Mass.). IL-6 levels were elevated with levels of 152 and 98 pg/mL prior to initiation of IL-7 therapy and subsequently decreased to 30 and 6 pg/mL. IL-1β, IL-2, IL-10, IL-17, and TNF-α were not significantly elevated at any timepoint. Interestingly, the patient maintained high levels of COVID-19-specific antibodies throughout his hospitalization (Leinco Technologies, St. Louis, Mo.) (TABLE 12).

Given his severity of illness at hospital presentation without significant comorbidities and reports of associated inborn errors of immunity, whole exome sequencing was performed. A heterozygote genetic variant in TICAM1 (p.S60C) was found. TICAM1 encodes for Toll/interleukin-1 receptor homology domain-containing adapter-inducing IFN-β, an adapter protein involved in TLR3 responses. The p.S60C loss of function variant was recently reported to associate with COVID-19 severity (^{19,20}). Enzyme-linked immunosorbent assays were performed for plasma type I IFN levels and demonstrated undetectable levels of all subtypes of IFN-α (R&D Systems, Minneapolis, Minn.) throughout his hospitalization, as well as IFN-β levels of 360 and 338 pg/mL prior to IL-7 treatment, and 130 and 4.1 pg/mL following treatment (TABLE 12). Both IFN-α and IFN-β levels in a cohort of seven healthy control subjects approached a mean of 0 pg/mL. IFN-α in 10 COVID-19 patients with disease severity comparable with that of the patient demonstrated levels ranging from 0 to 93 pg/mL; IFN-β levels from 50 to 811 pg/mL. Informed consent for publication was obtained from the patient.

TABLE 12
Plasma Cytokine and SARS-CoV-2 Antibody Levels.

| | Concentration | | | |
|---|---|---|---|---|
| | Pre IL-7, pg/mL | | Post IL-7, pg/mL | |
| Analytes | Day 5 | Day 7 | Day 12 | Day 21 |
| IL-1β | 0.16 | Undetectable | Undetectable | Undetectable |
| IL-2 | 0.25 | 0.17 | Undetectable | Undetectable |
| IL-6 | 152 | 98 | 30 | 6 |
| IL-10 | 3.55 | 5.53 | 2.71 | 0.83 |
| IL-12 | Undetectable | 0.03 | Undetectable | Undetectable |
| IL-17 | 0.11 | 0.16 | 0.14 | 0.12 |
| IFN-γ | 0.37 | 1.23 | Undetectable | Undetectable |
| Tumor necrosis | 0.05 | 0.04 | Undetectable | Undetectable |
| factor-α | | | | |
| IFN-α (all | Undetectable | Undetectable | Undetectable | Undetectable |
| subtypes) | | | | |
| IFN-β | 360 | 338 | 130 | 4.1 |
| Coronavirus | High positive | High positive | High positive | High positive |
| disease | | | | |
| immunoglobulin | | | | |
| G/M/A | | | | |

IFN = interferon, IL = interleukin.

Discussion

We demonstrate the use of IL-7 as an immunoadjuvant therapy in the treatment of COVID-19 disease. IL-7 not only restores lymphocyte counts, but reverses T cell exhaustion as evidenced by increased lymphocyte ex vivo IFN-γ production, essential for effective host immune response to pathogens. A previous report of compassionate use of IL-7 in critically ill COVID-19 disease patients with severe lymphopenia showed that IL-7 was safe, reversed the profound lymphopenia, and was well tolerated (²⁴⁻²⁶). Importantly, previous studies from our group reported that using the ex vivo stimulatory ELISpot assay in critically ill COVID-19 disease patients, we demonstrated that patients whose lymphocytes failed to produce IFN-γ upon stimulation trended toward mortality. Additionally, ex vivo stimulation of these patients' cells with IL-7 restored lymphocyte IFN-γ production (¹⁸).

Severity of disease in COVID-19 is often associated with a dysfunctional type I IFN (IFN-α and IFN-β) antiviral response. Several inborn errors of type I IFN signaling as well as autoantibodies have been identified in relation to severe cases, in addition to numerous additional cases without a known underlying cause (^{19,20}). Type I IFN signaling occurs locally in the lungs as well as systemically, to activate the innate and adaptive immune response to a viral pathogen. Defective type I IFN signaling enables unrelenting nuclear factor kappa-light-chain-enhancer of activated B cells-driven systemic inflammation with elevated circulating levels of IL-6 and TNF-α, promoting increased local tissue damage and multisystem organ dysfunction (²⁷). IL-6/signal transducer and activator of transcription-3 signaling additionally can cause an immunosuppressive phenotype with decreased antigen presentation by mononuclear cells and suppressed lymphocyte function (²⁸). Therefore, we hypothesize that the dysfunctional viral clearance observed in severe COVID-19 could be linked to decreased type I IFN signaling and unbalanced inflammation, leading to innate and adaptive immunosuppression. Our patient was found to be lacking in circulating plasma IFN-α, while also demonstrating elevated IL-6 levels and a dysfunctional ex vivo T cell response to stimulation. Immunotherapy with IL-7 improves cellular adaptive immune function, promoting proliferation and activation of effector T cells, which will improve viral clearance and restore immune homeostasis. The TICAM1 mutation is a defect in TLR3 signaling. IFN-γ (type II IFN) is the integral effector molecule downstream of TLR3. In a patient with defective TLR3 signaling, it is conceivable that essentially bypassing this receptor pathway using an immune stimulant such as recombinant human IL-7 could restore IFN-γ expression and therefore improved immune function and T cell activation during an acute infection (²⁹).

The presence of the loss of function mutation in TICAM1 shows the importance of the host response to COVID-19 infection. This patient was previously healthy, like those reported to have heterozygous mutations in genes involved in type I IFN responses. Having genetic information available may guide future therapies in critically ill patients as recently reported (30).

The patient described in this report maintained a robust anti-SARS-CoV-2 antibody response from day 5 post admission to day 21. Although some reports describe weak early antibody production to correlate with severity of disease (³¹), others describe no correlation between antibody levels and severity of disease, with a trend toward higher levels in more severe patients (³²). Antibody levels are unlikely to be positively predictive of outcomes, and therefore, immunotherapy to stimulate the T cell adaptive response should be considered.

Another noteworthy observation in this case is the precision medicine approach using a functional immune assay (ELISpot) to evaluate a patient's response and candidacy for an immune enhancing therapy (i.e., IL-7). For our patient, the ELISpot assay showed severe depression of adaptive immunity, indicating that a restorative therapy might be useful in restoring lymphocyte function. Previously, our group used the ELISpot assay to show that IL-7 restored lymphocyte function in critically ill COVID-19 disease patients. The present study advances the utility of such an approach, especially for precisely curated therapies applied to a patient's functional immune state. In this case, IL-7 administration improved both T cell IFN-γ production as well as the number of SARS-CoV-2-specific T cells.

In conclusion, administration of IL-7 to a critically ill COVID-19 patient markedly improved patient immunity and increased SARS-CoV-2-specific lymphocytes, thereby potentially enhancing viral elimination. Presently, IL-7 is available from RevImmune with an FDA expanded access protocol for compassionate use (EAP IND number 151107). Administration of IL-7 alone or in combination with other therapies warrants serious consideration for COVID-19 patients with evidence of immunosuppression.

### REFERENCES

1. Lim Z J, Subramaniam A, Ponnapa Reddy M, et al.: Case fatality rates for patients with COVID-19 requiring invasive mechanical ventilation. A meta-analysis. Am J Respir Crit Care Med. 2021; 203:54-66
2. Quah P, Li A, Phua J: Mortality rates of patients with COVID-19 in the intensive care unit: A systematic review of the emerging literature. Crit Care. 2020; 24:285
3. Auld S C, Caridi-Scheible M, Blum J M, et al.: ICU and ventilator mortality among critically ill adults with coronavirus disease 2019. Crit Care Med. 2020; 48:e799-e804
4. Grasselli G, Zangrillo A, Zanella A, et al.; COVID-19 Lombardy ICU Network. Baseline characteristics and outcomes of 1591 patients infected with SARS-CoV-2 admitted to ICUs of the Lombardy Region, Italy. JAMA. 2020; 323:1574-1581
5. Pepe M, Maroun-Eid C, Romero R, et al.: Clinical presentation, therapeutic approach, and outcome of young patients admitted for COVID-19, with respect to the elderly counterpart. Clin Exp Med. 2021; 21:249-268
6. COVID-ICU Group on behalf of the REVA Network and the COVID-ICU Investigators. Clinical characteristics and day-90 outcomes of 4244 critically ill adults with COVID-19: A prospective cohort study. Intensive Care Med. 2021; 47:60-73
7. Alharthy A, Aletreby W, Faqihi F, et al.: Clinical characteristics and predictors of 28-day mortality in 352 critically ill patients with COVID-19: A retrospective study. J Epidemiol Glob Health. 2021; 11:98-104
8. Yu K, He J, Wu Y, et al.: Dysregulated adaptive immune response contributes to severe COVID-19. Cell Res. 2020; 30:814-816
9. Blot M, Bour J B, Quenot J P, et al.; LYMPHONIE study group. The dysregulated innate immune response in severe COVID-19 pneumonia that could drive poorer outcome. J Transl Med. 2020; 18:457
10. Qin C, Zhou L, Hu Z, et al.: Dysregulation of immune response in patients with coronavirus 2019 (COVID-19) in Wuhan, China. Clin Infect Dis. 2020; 71:762-768
11. Esmaeilzadeh A, Elahi R: Immunobiology and immunotherapy of COVID-19: A clinically updated overview. J Cell Physiol. 2021; 236:2519-2543
12. Gavriatopoulou M, Ntanasis-Stathopoulos I, Korompoki E, et al.: Emerging treatment strategies for COVID-19 infection. Clin Exp Med. 2021; 21:167-179
13. Jamilloux Y, Henry T, Belot A, et al.: Should we stimulate or suppress immune responses in COVID-19? Cytokine and anti-cytokine interventions. Autoimmun Rev. 2020; 19:102567
14. Henry B, Cheruiyot I, Vikse J, et al.: Lymphopenia and neutrophilia at admission predicts severity and mortality in patients with COVID-19: A meta-analysis. Acta Biomed. 2020; 91:e2020008
15. Tan L, Wang Q, Zhang D, et al.: Lymphopenia predicts disease severity of COVID-19: A descriptive and predictive study. Signal Transduct Target Ther. 2020; 5:33
16. Wang D, Hu B, Hu C, et al.: Clinical characteristics of 138 hospitalized patients with 2019 novel coronavirus-infected pneumonia in Wuhan, China. JAMA. 2020; 323:1061-1069
17. Mazer M B, C Caldwell C, Hanson J, et al.: A whole blood enzyme-linked immunospot assay for functional immune endotyping of septic patients. J Immunol. 2021; 206:23-36
18. Remy K E, Mazer M, Striker D A, et al.: Severe immunosuppression and not a cytokine storm characterizes COVID-19 infections. JCI Insight. 2020; 5:140329
19. Zhang Q, Bastard P, Liu Z, et al.; COVID-STORM Clinicians; COVID Clinicians; Imagine COVID Group; French COVID Cohort Study Group; CoV-Contact Cohort; Amsterdam UMC Covid-19 Biobank; COVID Human Genetic Effort; NIAID-USUHS/TAGC COVID Immunity Group. Inborn errors of type I IFN immunity in patients with life-threatening COVID-19. Science. 2020; 370:eabd4570
20. Bastard P, Rosen L B, Zhang Q, et al.; HGID Lab; NIAID-USUHS Immune Response to COVID Group; COVID Clinicians; COVID-STORM Clinicians; Imagine COVID Group; French COVID Cohort Study Group; Milieu Intérieur Consortium; CoV-Contact Cohort; Amsterdam UMC Covid-19 Biobank; COVID Human Genetic Effort. Autoantibodies against type I IFNs in patients with life-threatening COVID-19. Science. 2020; 370:eabd4585
21. Wu W, Metcalf J P: The Role of Type I IFNs in influenza: Antiviral superheroes or immunopathogenic villains? J Innate Immun. 2020; 12:437-447
22. Verhoef P A, Kannan S, Sturgill J L, et al.: Severe acute respiratory syndrome-associated coronavirus 2 infection and organ dysfunction in the ICU: Opportunities for translational research. Crit Care Explor. 2021; 3:e0374
23. Mazer M B, Caldwell C C, Hanson J, et al.: A whole blood enzyme-linked immunospot assay for functional immune endotyping of septic patents. J Immunol. 2021; 206:23-36
24. Laterre P F, Francois B, Collienne C, et al.: Association of interleukin 7 immunotherapy with lymphocyte counts among patients with severe coronavirus disease 2019 (COVID-19). JAMA Netw Open. 2020; 3:e2016485
25. Mackall C L, Fry T J, Gress R E: Harnessing the biology of IL-7 for therapeutic application. Nat Rev Immunol. 2011; 11:330-342
26. Monneret G, de Marignan D, Coudereau R, et al.: Immune monitoring of interleukin-7 compassionate use in a critically ill COVID-19 patient. Cell Mol Immunol. 2020; 17:1001-1003
27. Hadjadj J, Yatim N, Barnabei L, et al.: Impaired type I interferon activity and inflammatory responses in severe COVID-19 patients. Science. 2020; 369:718-724
28. Ohno Y, Kitamura H, Takahashi N, et al.: IL-6 down-regulates HLA class II expression and IL-12 production of human dendritic cells to impair activation of antigen-specific CD4(+) T cells. Cancer Immunol Immunother. 2016; 65:193-204
29. Negishi H, Osawa T, Ogami K, et al.: A critical link between Toll-like receptor 3 and type II interferon signaling pathways in antiviral innate immunity. Proc Natl Acad Sci USA. 2008; 105:20446-20451
30. Levy R, Bastard P, Lanternier F, et al.: IFN-α2a therapy in two patients with inborn errors of TLR3 and IRF3 Infected with SARS-CoV-2. J Clin Immunol. 2021; 41:26-27
31. Lenti M V, Aronico N, Pellegrino I, et al.: Depletion of circulating IgM memory B cells predicts unfavourable outcome in COVID-19. Sci Rep. 2020; 10:20836
32. Tan A T, Linster M, Tan C W, et al.: Early induction of functional SARS-CoV-2-specific T cells associates with rapid viral clearance and mild disease in COVID-19 patients. Cell Rep. 2021; 34:108728

What is claimed is:

1. A method of immune phenotyping a subject comprising:
a. providing or having been provided a biological sample from the subject;
b. stimulating a T cell or monocyte cell or both to secrete a cytokine associated with cellular immunity; and
c. quantitating at least one cytokine associated with cellular immunity using ELISpot assay or FluoroSpot assay in the biological sample.

2. The method of claim 1, further comprising
d. determining that a subject has an immunosuppressive endotype if the cytokine associated with cellular immunity is a proinflammatory cytokine and proinflammatory cytokine production or secretion is decreased compared to a control; or
d. determining that a subject has a hyper-inflammatory endotype if the cytokine associated with cellular immunity is a proinflammatory cytokine and the proinflammatory cytokine production or secretion is increased compared to a control.

3. The method of claim 1, further comprising determining if the subject has immunosuppressive endotype if immune cells amount is reduced compared to a control or hyper-inflammatory endotype if cytokine production is increased compared to a control.

4. The method of claim 1, further comprising detecting a level of innate immunity comprising detecting a level of blood monocytes or detecting a level of low-density granulocytes or detecting a level of monocyte function or low-density granulocyte function.

5. The method of claim 1, further comprising detecting a level of adaptive cellular immunity comprising detecting a level of blood lymphocytes or blood lymphocytes function.

6. The method of claim 1, wherein the subject has an immunosuppressive endotype if an amount of CD4⁺ and CD8⁺ T cells is reduced compared to a control, has reduced responsiveness of the T cells to T cell receptor activation, or both.

7. The method of claim 1, wherein the cytokine associated with cellular immunity is a proinflammatory cytokine selected from the group consisting of T cell interferon-gamma (IFN- [symbol] ), monocyte tumor necrosis factor alpha (TNF-α), and combinations thereof.

8. The method of claim 1, wherein the cytokine associated with cellular immunity is selected from IFN- [symbol] , TNF-α, IL-1β, IL-6, IL-7, IL-8, IL-10, IL-12, MCP-1, IL-1RA, and any combination thereof.

9. The method of claim 1, wherein quantitating cytokines associated with cellular immunity comprises:
detecting an amount of cytokine-producing immune effector cells; or
detecting an amount of cytokine produced on a cell.

10. The method of claim 1, wherein quantitating cytokines associated with cellular immunity is measured in units of response per volume of blood.

11. The method of claim 1, wherein the biological sample comprises:
whole blood;
diluted whole blood;
circulating peripheral blood;
whole blood diluted in about a 1:1 ratio with PBS;
T cells or monocytes or both; or
plasma, leukocytes, red blood cells (RBCs), white blood cells (WBCs), platelets, cytokines, chemokines, or combinations thereof.

12. The method of claim 1, wherein the biological sample does not comprise isolated peripheral blood mononuclear cells (PBMCs).

13. The method of claim 1, further comprising:
evaluating adaptive and innate immune status;
evaluating monocyte or leukocyte function;
evaluating progression of immune dysfunction in a subject;
evaluating an effect of an immune therapy to restore innate and adaptive immunity in an immunosuppressed patient, optionally an immuno-adjuvant therapy to enhance host immunity;
identifying optimal immune therapy for use in a subject; or
improving immune function in a subject.

14. The method of claim 1, wherein
the subject has, is suspected of having, or is at risk for developing sepsis, autoimmune disease, autoimmunity, or cancer;
the subject has Fungal Wound Sepsis;
the subject has lymphopenia (≤1100 cells/μL);
the subject has undergone organ transplantation; or
the subject is in critical care.

15. The method of claim 1, wherein step b comprises measuring ex vivo cytokine production as a response to external stimuli.

16. The method of claim 1, wherein the subject is septic and is determined to be at risk for premature death if:
an amount of proinflammatory cytokine producing immune effector cells are decreased compared to a control; or
an amount of proinflammatory cytokine produced per cell measured by spot intensity are decreased compared to a control.

17. The method of claim 2, wherein
if the subject does not have an immunosuppressive endotype or the subject has a hyper-inflammatory endotype, the subject is administered a drug that blocks proinflammatory cytokines or inhibits an inflammatory signaling cascade;
if the subject has an immunosuppressive endotype, then the subject is administered IL-7 to restore disease-induced T cell exhaustion;
if the subject has sepsis and has an immunosuppressive endotype, a drug restoring immunity is administered to the subject;
if the subject is septic and immunosuppressed, then the subject is not administered corticosteroid therapy, optionally dexamethasone;
the subject has sepsis and has the immunosuppressive endotype, the subject is at risk for death;
if the subject has the immunosuppressive endotype, the subject is treated with immuno-modulatory drug therapies or immune adjuvants that enhance host immunity;
if the subject has an immunosuppressive endotype, then the subject is administered a checkpoint inhibitor or γ-chain cytokine that stimulate CD4 and CD8 T cells, optionally IL-17;
if the subject has a hyper-inflammatory endotype or does not have an immunosuppressive endotype, the subject is treated with drugs to inhibit a host inflammatory response;
if cytokine production in the subject is elevated, the subject is not treated with immunostimulant therapy; or
if cytokine production in the subject is elevated, the subject is treated with anti-cytokine therapy or drugs to negatively modulate an inflammatory response.

18. The method of claim 2, further comprising detecting an immunosuppressive endotype or a hyper-inflammatory endotype during progression of a disease, disorder, or condition or during treatment of a disease, disorder, or condition.

19. The method of claim 1, further comprising administering a drug to a subject in need thereof and determining immune function or leukocyte function of the subject in response to the drug, optionally, during a course of immune therapy.

20. The method claim 1, wherein
the subject has sepsis, COVID-19, cancer, trauma, or autoimmune disease;
the subject is a critically ill nonseptic (CINS) or post-transplant patient; or
the subject is immunosuppressed or a pediatric patient.

21. The method of claim 1, wherein the biological sample is placed in fluid contact with a test therapeutic agent, optionally cytokines/chemokines, IL6, anti-PD-1, anti-PD-L1, GM512, CSF, IL-7.

22. The method of claim 1, wherein the assay comprises a well pre-coated with a treatment directed at detecting one or more cytokines or chemokines.

23. A method of screening a test therapeutic agent comprising:
a. providing or having been provided an immune cell;
b. optionally determining if the immune cell has an immunosuppressive or hyper-inflammatory endotype;
c. contacting the immune cell with a test therapeutic agent; and
d. determining if one or more cytokines associated with cellular immunity are increased, decreased, or the same compared to a control or compared to before the immune cell was contacted with the test therapeutic agent.

24. The method of claim 23, wherein the immune cell is a leukocyte, a monocyte, a T cell, or a combination thereof.

25. The method of claim 23, wherein the test therapeutic agent is an immune adjuvant that selectively targets an immune effector cell type.

26. The method of claim 23, wherein the test therapeutic agent is an immune adjuvant selected from anti-PD-1, anti-PD-L1, OX-40, GM-CSF, and IL-7.

27. The method of claim 23, wherein the one or more cytokines associated with cellular immunity is T cell IFN-γ, monocyte TNF-α, or a combination thereof.

28. The method of claim 23, wherein the immune cell are obtained from a subject having sepsis, COVID-19, cancer, trauma, autoimmune disease, or a critically ill nonseptic (CINS) or post-transplant patient.

29. A method of evaluating drug efficacy by measuring immune function in a subject:
a. providing or having been provided a biological sample comprising whole blood or diluted whole blood or isolated peripheral blood mononuclear cells (PBMCs);
b. quantitating T cell interferon-gamma (IFN- [symbol] ) and monocyte TNF-α production using ELISpot in the biological sample comprising whole blood or diluted whole blood;
c. optionally determining that a subject has an immunosuppressive endotype if T cell cytokine or monocyte cytokine production is decreased compared to a control; and
d. administering a drug to the subject and determining the immune function of the subject in response to the drug.

30. The method of claim 29, wherein the T cell cytokine is interferon-gamma (IFN- [symbol] ).

31. The method of claim 29, wherein the monocyte cytokine is selected from one or more of TNF-α, IL-2, IL-6, and IL-12.

32. The method of claim 29, wherein
the subject has sepsis, COVID-19, cancer, trauma, or autoimmune disease;
the subject is a critically ill nonseptic (CINS) or post-transplant patient; or
the subject is immunosuppressed or a pediatric patient.

33. An ELISpot or FluorSpot assay comprising wells, wherein the wells are precoated, resulting in precoated wells, with one or more test therapeutic agents or one or more cytokine or chemokine detecting agents.

34. The ELISpot or FluorSpot assay of claim 33, wherein the one or more test therapeutic agents are tocilizumab, haptoglobin, hemopexin, ox40, IL7, or steroids.

35. The ELISpot or FluorSpot assay of claim 33, further comprising a biological sample in fluid contact with the precoated wells, wherein the biological sample comprises whole blood, diluted whole blood, or isolated immune cells.

36. The assay of claim 35, wherein the biological sample is obtained from
a subject having or suspected of having sepsis, COVID-19, cancer, trauma, or autoimmune disease;
a critically ill nonseptic (CINS) subject or post-transplant patient; or
an immunosuppressed or a pediatric patient.

37. The assay of claim 33, wherein the assay produces accelerated results compared to a PBMC assay.

38. A method of reversing lymphopenia or improving T cell function in a subject comprising:
a. providing or having been provided a biological sample from the subject;
b. stimulating a T cell or monocyte cell or both to secrete a cytokine associated with cellular immunity;
c. quantitating at least one cytokine associated with cellular immunity using ELISpot assay or FluoroSpot assay in the biological sample; and
d. administering an immune-stimulating agent, optionally IL-7, GM-CSF, anti-PD-1, anti-PD-L1, and OX-40 agonistic Abs.

39. The method of claim 38, wherein
the subject has sepsis, COVID-19, cancer, trauma, or autoimmune disease;
the subject is a critically ill nonseptic (CINS) or post-transplant patient; or
the subject is immunosuppressed or a pediatric patient.

40. A kit comprising an ELISpot or FluoroSpot assay comprising test agent-coated wells or wells coated with cytokine or chemokine detecting agents; and optionally a biological sample comprising whole blood or PBMCs.
